(19) United States
(12) Patent Application Publication
(10) Pub. No.: US 2021/0265034 A1
(43) Pub. Date: Aug. 26, 2021

(54) SYSTEMS AND METHODS FOR REFINING A DIETARY TREATMENT REGIMEN USING RANKED BASED SCORING

(71) Applicant: Shaklee Corporation, Pleasanton, CA (US)

(72) Inventor: Erin Barrett, Pleasanton, CA (US)

(21) Appl. No.: 17/178,051

(22) Filed: Feb. 17, 2021

Related U.S. Application Data
(60) Provisional application No. 62/978,158, filed on Feb. 18, 2020.

(51) Int. Cl. G16H 20/60; G16H 10/20; G16H 50/20; G06Q 30/02; G06Q 30/06; G16H 50/30; G16H 10/60; G16H 40/20; G16H 20/30; G06Q 10/08; G06F 16/901

(52) U.S. Cl. CPC G16H 20/60; G16H 10/20; G16H 50/20; G06Q 30/0203; G06Q 30/0631; G06Q 2230/00; G16H 10/60; G16H 40/20; G16H 20/30; G06Q 10/083; G06F 16/9017; G16H 50/30

(57) ABSTRACT

Systems and methods for providing a dietary recommendation are provided. Assessment responses are obtained for a survey. From the responses a set of tags is identified and polled against decision rules. Each firing of a decision rule casts a weighted or unweighted vote against one or more nutritional products. A subset of products satisfying satisfy a nutritional product selection criterion is identified from the votes. The subset of products is filtered against periodic nutritional limits specifying a maximum consumable amount of a supplement given one or more physiological characteristics specified by the responses. The filtering independently sums the amount of each dietary supplement and removes from one or more doses of a product when a nutritional limit is exceeded. This subset is provided as a dietary recommendation to the subject.

### CROSS REFERENCE TO RELATED APPLICATIONS

This application claims priority to U.S. Provisional Patent Application No. 62/978,158, filed on Feb. 18, 2020, the contents of which is hereby incorporated by reference in its entirety for all purposes.

### TECHNICAL FIELD

The present disclosure relates generally to systems and methods for providing a dietary recommendation.

### BACKGROUND

Since the turn of this century, sales for nutritional products, such as dietary supplements, in the United States have consistently increased, surpassing $30 billion dollars in 2011. Nutrition Business Journal, 2012, “Considering a Post-DSHEA World,” print. This increase in sales is based on both an increase in consumers for nutritional products, as well as the number of nutritional products utilized by consumers. Bailey et al., 2013, “Why US Adults Use Dietary Supplements,” JAMA Internal Medicine, 173(5), pg. 355. Surveys indicate that approximately half of these nutritional product consumers are adults that consume at least one nutritional product on a regular basis. Blendon et al., 2001, “Americans' Views on the Use and Regulation of Dietary Supplements,” Archines of Internal Medicine, 161(6), pg. 805.

While adults are consuming nutritional products, such as dietary supplements, at historically high rates, personal motivations and reasoning for consuming nutritional products appears unguided and arbitrary. Bailey et al, 2013, “Why US Adults Use Dietary Supplements,” JAMA Internal Medicine, 173(5), pg. 355. For instance, professional medical practitioners personally recommended less than a quarter of consumed nutritional products. Id. Moreover, most of these professionally recommended nutritional products were either calcium or a general multi-vitamin, leaving potential nutritional voids that are unfulfilled by a general multi-vitamin. Id. Interestingly, only a fifth of nutritional product consumers used a supplement to “supplement the diet,” while most others used nutritional products, such as dietary supplements, to either improve or maintain their overall health. Furthermore, users frequently reported a wide range of motivation for consuming a specific dietary supplement, often unrelated to scientifically proven benefits of the specific dietary supplement. Id. Accordingly, specific reasons and motivations for each user to consume a specific nutritional product is often controversial or conflicting with the personal beliefs of the user.

One approach to making nutritional products more accessible is to provide users with directed surveys that, responsive to user answers, make nutritional product recommendations. Conventionally, these surveys are provided to a population of users as a predetermined questionnaire, with each user being provided the same survey. The surveys are designed to pose general dietary and health related questions to the user. The questions guide the user towards one of a selection of predetermined dietary recommendations, pigeonholing the user into a specific recommendation without tailoring the recommendation to the needs of each specific user. Without this tailoring to the specific user, there exists a risk that the provided nutritional product recommendation will have an adverse effect on the user, such as recommending the user consume a specific nutritional product in excess of a recommended dietary supplement allowance. Furthermore, these predetermined nutritional product recommendations fail to yield specific useful information to each user as to why each specific nutritional product recommendation is provided to the user and how the user recommendation is beneficial to the user. Instead, the recommendations provide a broad, blanket statement untethered to the needs of the specific user.

Thus, what is needed in the art is to overcome the difficulty of providing personalized nutritional product recommendations to a population of users that not only addresses the nutritional product needs of individual users but also provides personalized information describing various beneficial aspects of the personalized dietary recommendation.

The information disclosed in this Background section is only for enhancement of understanding of the general background of the invention and should not be taken as an acknowledgement or any form of suggestion that this information forms the prior art already known to a person skilled in the art.

### SUMMARY

The present disclosure provides improved systems and methods for generating dietary recommendations. Subjects are provided dietary recommendation in the form of specific nutritional product recommendations, drawn from a diverse selection of nutritional products, tailored to their specific needs based on survey results they complete. Such dietary recommendations provide each subject with a selection of nutritional products that meet their individual nutritional needs, e.g., their needs with respect to macronutrients, vitamins, and minerals. Further, the tailoring of the dietary recommendation accounts for various dietary restrictions, dietary and/or personal preferences, and health goals of the user, allowing the dietary recommendation to coordinate with the lifestyle, or desired lifestyle, of the user. Additionally, the tailoring of the dietary recommendation allows the subject to receive information for improving user health through the dietary recommendation. This is advantageous because complex dietary regimens can be customized and offered to populations of users with optimized selections of nutritional products.

In the present disclosure, a subject completes an assessment survey. In so doing, the subject provides a plurality of assessment responses. Such assessment responses are responsive to questions regarding the subject. Non-limiting examples of such questions pertain to the subject's age, sex, height, weight, blood pressure, familial health condition history, lifestyle, and/or health goals, etc. Each respective assessment response provided by the subject is used to select a corresponding set of tags associated with the respective assessment response according to an assessment response to tag lookup data structure. In this way, a plurality of tags is collectively identified for the user across the various assessment responses provided by the subject. Non-limiting examples of such tags include tags relating to certain vitamins (vitamin tags), tags relating to certain minerals (mineral tags), specialty tags, and functional tags. For instance, a particular assessment response provided by the subject may trigger the vitamin tag “vitamin A,” the mineral tag “calcium,” the specialty tag “glucosamine,” and/or the functional tag “fiber.” Thus, each corresponding set of tags selected by each respective assessment response forms a plurality of tags. Next, the plurality of tags is collectively polled against a plurality of decision rules. Each respective decision rule fires when the plurality of tags contains a specific tag or a specific combination of tags associated with the respective decision rule. For instance, a first example decision rule fires when the plurality of tags includes the vitamin tag “vitamin A.” A second example decision rule fires when the plurality of tags includes both the vitamin tag “vitamin A” and the mineral tag “calcium.” Each time a determination is made that the first plurality of tags includes the one or more tags independently associated with a respective decision rule in the plurality of decision rules, the respective decision rule is fired thereby casting a weighted or unweighted vote associated with the respective decision rule against one or more nutritional products in a plurality nutritional products specified by the respective decision rule. For instance, the first example decision rule presented above, which fires when the plurality of tags includes the vitamin tag “vitamin A,” may cast a vote for a first nutritional product when fired while the second example decision rule presented above, which fires when the plurality of tags includes both the vitamin tag “vitamin A” and the mineral tag “calcium,” may cast one vote for the first nutritional product as well as another vote for a second nutritional product when fired. In this way, two or more nutritional products in the plurality of nutritional products each collect one or more weighted or unweighted votes upon polling the plurality of tags against the plurality of decision rules. This is highly advantageous because the votes for the nutritional products are responsive to self-assessments made by the subject and thus are designed to fulfill the needs identified by the subject through such self-assessment. However, the advantages of the present disclosure do not stop there. The present disclosure ensures that the collection of nutritional products that received votes is not too numerous. To this end, only those nutritional products whose votes satisfy a nutritional product selection criterion are retained for a draft dietary recommendation to be delivered to the subject. For example, in some instances, satisfaction of the nutritional product selection criterion requires that a nutritional product receive at least a threshold number of votes, or that the nutritional product is among the top X nutritional products in terms of number of votes received, where X is a predetermined positive integer (e.g., among the top 2 nutritional products in terms of number of votes, among the top 3 nutritional products in terms of number of votes, etc.). The present disclosure advantageously further ensures that consumption of the selected nutritional products in the draft dietary recommendation will not cause the subject to take more than the recommended dose of any of the various dietary supplements within the selected nutritional products. This is done by filtering the nutritional products in the draft dietary recommendation against a plurality of periodic nutritional limits. Each of these periodic nutritional limits specifies a corresponding maximum amount of a corresponding dietary supplement that can be consumed in a corresponding period of time given one or more physiological characteristics of the subject specified in the plurality of assessment responses. A “dietary supplement” can be a particular nutrient, a particular nutritional product, a chemical, or any other composition for which nutritional limit data is available. In this way, for each respective dietary supplement associated with each periodic nutritional limit in the plurality of periodic nutritional limits, the corresponding amount of respective dietary supplement found across the nutritional products in the draft dietary recommendation for the subject is determined. When one or more periodic nutritional limits in the plurality of periodic nutritional limits identifies that a periodic nutritional limit for a dietary supplement has been exceeded by the draft dietary recommendation, one or more doses of one or more nutritional products is removed from the draft dietary recommendation in order to prevent the one or more periodic nutritional limits from being exceeded. Two example use cases illustrate such filtering. In the first example use case, a periodic nutritional limit specifies that only twenty grams of nutritional product A may be consumed by the subject but the draft dietary recommendation specifies consumption of forty grams of product A per week. In this instance, the draft dietary recommendation is revised to lower the recommendation consumption of nutritional product A to twenty grams per week. In the second example use case, another periodic nutritional limit specifies that no more than 100 mg of iron may be consumed per day. The draft dietary recommendation includes two nutritional products (nutritional product A and nutritional product B) that have iron. The draft dietary recommendation recommends consuming one gram of nutritional product A per day which contains 100 mg of iron. The draft dietary recommendation further recommends consuming ten gram of nutritional product B per day which also contains 100 mg of iron per day. In this instance, product A or product B will be removed from the final dietary periodic recommendation. For instance, between products A and B, the product that received fewer votes may be removed from the final dietary product recommendation. Finally, the final dietary recommendation, comprising recommended dosage (amounts) of selected dietary products that satisfied the nutritional product selection criterion and the one or more periodic nutritional limits are provided to the subject.

Accordingly, turning to more specific aspects of the present disclosure, systems and methods for generating dietary recommendations that account for the subject's individual dietary needs and goals are provided. In some embodiments, the methods and systems described herein include receiving responses for a survey from a subject. The responses are associated with a set of tags that are polled against decision rules. If a decision rule is fired, a vote is cast for one or more nutritional products associated with the decision rule. A subset of products satisfying satisfy a criterion is identified from the votes. The subset of products is filtered against various nutritional limits to ensure proper dosages and prevent nutritional redundancy. The filtering independently sums the amount of each dietary supplement and removes one or more doses of a product when a nutritional limit is exceeded. This subset is provided as a dietary recommendation to the subject.

In more detail, one aspect of the present disclosure provides a method of providing a dietary recommendation to a subject. The method occurs at a computer system. The computer system includes at least one processor and a memory storing at least one program for execution by the at least one processor. The at least one program include instructions for the method. A plurality of assessment responses is obtained in electronic form for an assessment survey presented to the subject. Each respective assessment response, in all or a subset of the plurality of assessment responses, is utilized to select a corresponding set of tags. Each tag in the set of tags is associated with the respective assessment response according to an assessment response to tag lookup data structure. Accordingly, a first plurality of tags is collectively identified. The first plurality of tags is polled against a plurality of decision rules. Each respective decision rule in the plurality of decision rules is independently associated with one or more tags in a second plurality of tags. Further, at least one tag in the second plurality of tags is incorporated into two or more decision rules in the plurality of decision rules. Moreover, the second plurality of tags includes all the tags in the first plurality of tags. Each time the polling determines that the first plurality of tags includes the one or more tags independently associated with a respective decision rule in the plurality of decision rules, the respective decision rule is fired. This firing of the respective decision rule casts a weighted or unweighted vote associated with the respective decision rule against one or more nutritional products in a plurality of nutritional products specified by the respective decision rule. As such, the firing causes two or more nutritional products in the plurality of nutritional products to have one or more weighted or unweighted votes upon polling all the tags in the first plurality of tags. A subset of the plurality of nutritional products is identified on the basis of the weighted or unweighted votes received by respective nutritional products in the plurality of nutritional products. The subset of the plurality of nutritional products consists of those nutritional products in the plurality of nutritional products that each received a sufficient number of weighted or unweighted votes by the polling to satisfy a nutritional product selection criterion. The subset of nutritional products is filtered against a plurality of periodic nutritional limits. Each respective periodic nutritional limit in the plurality of periodic nutritional limits specifies a corresponding maximum amount of a corresponding dietary supplement that can be consumed in a corresponding period of time given one or more physiological characteristics of the subject specified in the plurality of assessment responses. Further, the filtering independently sums, for each respective dietary supplement associated with each periodic nutritional limit in the plurality of periodic nutritional limits, the corresponding amount of respective dietary supplement in the subset of products. Moreover, the filtering removes one or more doses of one or more nutritional products from the subset of nutritional products when one or more periodic nutritional limits in the plurality of periodic nutritional limits is determined to have been exceeded. This removing one or more doses prevent the one or more periodic nutritional limits from being exceeded. After the filtering, the subset of nutritional products is used to provide a dietary recommendation to the subject.

In some embodiments, the casted weight either increases a contribution or decreases the contribution of the corresponding one or more nutritional products to satisfy the nutritional product selection criteria. In some embodiments, the casted weight against the one or more nutritional products is determined by a first assessment response in the plurality of assessment responses from the subject.

In some embodiments, in accordance with a determination that the subset of the plurality of nutritional products satisfies a threshold periodic nutritional limit, the identifying further includes identifying a first product in the combination of one or more products.

In some embodiments, in accordance with a determination that the subset of the plurality of nutritional products satisfies a threshold score of unique tags, the subset of the plurality of nutritional products includes a first product.

In some embodiments, each product in the plurality of product is further associated with a respective classification in a plurality of classifications. Accordingly, the identifying further includes assigning a respective classification from a plurality of classifications to the subset of the plurality of nutritional products. Each product in the subset of the plurality of nutritional products is associated the respective classification of the subset of the plurality of nutritional products.

In some embodiments, the identifying further includes filtering the subset of the plurality of nutritional products based on one or more tags associated with a respective assessment response in the plurality of assessment responses.

In some embodiments, in accordance with a determination that the first plurality of tags includes a first tag and a second tag, the subset of the plurality of nutritional products includes a first product. In some embodiments, in accordance with a determination that the first plurality of tags includes a first tag but not a second tag, the combination of one or more products includes a first product.

In some embodiments, the filtering further includes evaluating each product in the subset of the plurality of products against a dimensional constraint associated with one or more products in the subset of the plurality of products.

In some embodiments, each assessment response in the plurality of assessment responses for the assessment survey is obtained from the subject sequentially thus forming the plurality of assessment responses. Accordingly, after each instance of the obtaining a respective assessment response, the method includes conducting the polling, the identifying, and the filtering. Further, in accordance with a determination that the plurality of assessment responses provided by the subject fail to satisfy the nutritional product selection criterion, the method includes repeating the obtaining for a proceeding assessment prompt to the respective assessment prompt in the assessment survey. Moreover, in accordance with a determination that the plurality of responses provided by the subject satisfy the threshold criteria, the dietary recommendation is provided.

In some embodiments, the second plurality of tags further includes a plurality of vitamin tags, a plurality of mineral tags, a plurality of specialty tags, a plurality of functional tags, or a combination thereof.

In some embodiments, each tag in either of the plurality of vitamin tags or the plurality of mineral tags further includes a first periodic nutritional limit in the plurality of period nutritional limits that provides a recommended dosage of the corresponding dietary supplement.

In some embodiments, one or more tags in either of the plurality of vitamin tags or the plurality of mineral tags further includes a second periodic nutritional limit in the plurality of period nutritional limits that provides a threshold dosage of the corresponding dietary supplement.

In some embodiments, the filtering further includes, in accordance with a determination that one or more periodic nutritional limits in the plurality of periodic nutritional limits is exceeded, substituting at least a first product in the subset of the plurality of nutritional products for a second product in the plurality of nutritional products.

In some embodiments, the plurality of vitamin tags includes a folic acid tag, a riboflavin tag, a vitamin A tag, a vitamin B6 tag, a vitamin B12 tag, a vitamin C tag, a vitamin D tag, a vitamin E tag, a vitamin K tag, or a combination thereof.

In some embodiments, the plurality of mineral tags includes a calcium tag, an iron tag, a magnesium tag, a potassium tag, and a zinc tag.

In some embodiments, the plurality of specialty tags includes an ashwaganda tag, a black cohosh tag, a boswellia tag, a carotenoids tag, a catechins tag, a chromium tag, a ubiquinone (e.g., coenzyme Q-10) tag, a flavonoids tag, a glucosamine tag, a gamma lionlenic acid (GLA) tag, a L-Theanine tag, a L-Tyrosine tag, a nitrate tag, an omega-3 fatty acid tag, a polyphenols tag, a probiotics tag, a plant sterol/stanol tag, a procyanidins tag, a saw palmetto tag, a St. John's wort tag, a tart cherry extract tag, or a combination thereof.

In some embodiments, the plurality of functional tags includes a caffeine tag, an electrolyte tag, a fiber tag, a greens tag, a menthol tag, a prebiotic tag, a protein tag, or a combination thereof.

In some embodiments, the protein tag further includes a form tag providing a dosage form of the protein dietary supplement, a source tag providing a source of the protein dietary supplement, or a combination thereof.

In some embodiments, the assessment survey presented to the subject includes a plurality of assessment prompts that elicit the one or more physiological characteristics of the subject, the plurality of assessment prompts including a plurality of biometric assessment prompts, a plurality of life-stage assessment prompts, a plurality of physiological assessment prompts, a plurality of dietary assessment prompts, a plurality of lifestyle assessment prompts, a plurality of behavioral assessment prompts, a plurality of health goal assessment prompts, or a combination thereof.

In some embodiments, the plurality of biometric assessment prompts elicits a corresponding plurality of assessment responses including an age of the subject, a sex of the subject, a height of the subject, and a weight of the subject.

In some embodiments, in accordance with a determination that one or more respective assessment responses obtained from the subject indicates an age in a range of from 20 years of age to 45 years of age and a sex of female, the plurality of life-stage assessment prompts elicit a response including whether the subject is pregnant, breastfeeding, or has an experience of physical or emotional symptoms.

In some embodiments, in accordance with a determination that one or more assessment responses obtained from the subject indicates an age greater than 45 years of age and a sex of female, the plurality of life-stage assessment prompts further elicit a response including whether the subject has an experience of physical or emotional symptoms or menopause related symptoms.

In some embodiments, the plurality of physiological assessment prompts elicit a corresponding plurality of assessment responses including whether a health practitioner associated with the subject has indicated a concern associated with the subject for one or more health conditions, a familial health condition history, whether the health practitioner associated with the subject has indicated a recommendation for one or more dietary supplements, and whether the subject is currently taking a pharmaceutical composition.

In some embodiments, the one or more health conditions of concern includes a cholesterol level, the weight of the subject, a blood sugar level of the subject, a blood pressure level of the subject, a bone health metric of the subject, or a combination thereof.

In some embodiments, in accordance with a determination that one or more assessment responses obtained from the subject indicates the blood sugar level health condition concern or the weight health condition concern, the plurality of physiological assessment prompts further elicit a response including a diabetes status of the subject.

In some embodiments, the familial health condition history including an indication of a familial history of high cholesterol, a familial history of cardiovascular disease, a familial history of high blood pressure, a familial history of osteoporosis, or a combination thereof.

In some embodiments, the dietary recommendation for the subject includes a recommendation for vitamin D, an omega-3 fatty acid, iron, a probiotic, a multivitamin, or a combination thereof.

In some embodiments, the pharmaceutical composition indicated by the subject includes a cholesterol lowering pharmaceutical composition, a blood thinning pharmaceutical composition, a blood pressure pharmaceutical composition, an acid-suppressing pharmaceutical composition, or a combination thereof.

In some embodiments, the plurality of dietary assessment prompts elicit a corresponding plurality of assessment responses including whether the subject has one or more dietary restraints, a number of vegetable servings consumed by the subject, a number of fruit servings consumed by the subject, a number of diary servings consumed by the subject, a number of omega-3 fatty acid servings consumed by the subject.

In some embodiments, the one or more dietary restraints include an indication if the corresponding restraint is a personal preference of the subject or a medical requirement.

In some embodiments, the one or more dietary restraints include a vegetarian restrain, a vegan restraint, a pescatarian restraint, a gluten intolerance restraint, a dairy intolerance restraint, a nut intolerance restraint, a soy intolerance restraint, a kosher restraint, a ketogenic restraint, a paleolithic restraint, a caffeine restraint, or a combination thereof.

In some embodiments, the plurality of life-style assessment prompts elicit a corresponding plurality of assessment responses including a level of physical activity endured by the subject, whether the subject physical exerts themselves at a work, an energy level of the subject, a stress level of the subject, a sleeping habit of the subject, a cognitive health assessment of the subject, a sun exposure level of the subject, a computer use level of the subject.

In some embodiments, in accordance with a determination that one or more assessment responses obtained from the subject satisfies a first threshold level of physical activity, the plurality of life-style assessment prompts further elicit an assessment response including an indication of a type of physical activity endured by the subject.

In some embodiments, in accordance with a determination that one or more assessment responses obtained from the subject satisfies a second threshold level of physical activity, the plurality of life-style assessment prompts further elicit an assessment response including an indication of a desired health goal from consuming a product in the plurality of products.

In some embodiments, the desired health goal includes optimizing a performance of the physical activity, optimizing an aspect of muscle growth, reducing an aspect of muscle soreness, replenishing one or more dietary supplements, or a combination thereof.

In some embodiments, the plurality of behavioral assessment prompts elicits a corresponding plurality of assessment responses including whether the subject is currently taking a nutritional supplement.

In some embodiments, in accordance with a determination that one or more assessment responses obtained from the subject indicates that the subject is currently taking a nutritional supplement, the plurality of behavioral assessment prompts further elicit an assessment response including a number of nutritional supplements that the subject is currently taking.

In some embodiments, in accordance with a determination that one or more assessment responses obtained from the subject indicates the subject is currently taking a nutritional supplement, the plurality of behavioral assessment prompts further elicit an assessment response including a role of the nutritional supplement the subject is currently taking.

In some embodiments, the plurality of health goal assessment prompts elicits a corresponding plurality of assessment responses including an indication of one or more health related interest.

In some embodiments, the indication of one or more health related areas of interest includes a ranking of the one or more health related interest.

In some embodiments, the assessment survey is sequentially provided to the subject.

In some embodiments, in accordance with a determination that a respective assessment response obtained from the subject is a first assessment response, bypassing the obtaining for a proceeding assessment prompt to the respective assessment prompt in the assessment survey, which forms the plurality of assessment responses.

In some embodiments, the obtaining further includes obtaining a plurality of anthropometric data associated with the subject.

In some embodiments, the plurality of anthropometric data is derived from a biological sample obtained from the subject.

In some embodiments, the plurality of anthropometric data further includes one or more subject preferences associated with the subject of the client device.

In some embodiments, the plurality of anthropometric data further includes a plurality of genetic data and/or a plurality of metabolomics data.

In some embodiments, the plurality of assessment responses is determined by the subject.

In some embodiments, the filtering further includes determining a body mass index (BMI) of the subject and the nutritional product selection criterion accounts for the BMI of the subject.

In some embodiments, the BMI of the subject is based on one or more assessment responses obtained from the subject indicating a weight of the subject and a height of the subject.

In some embodiments, the filtering further includes determining a target caloric intake of the subject and the nutritional product selection criterion accounts for the target caloric intake of the subject.

In some embodiments, the target caloric intake of the subject is based on one or more assessment responses obtained from the subject including a weight of the subject, a height of the subject, a sex of the subject, an age of the subject, and a level of physical activity endured by the subject.

In some embodiments, the obtaining further includes obtaining a physical location associated with the subject. Accordingly, the method further includes shipping the subset of the plurality of nutritional products of the dietary recommendation to the physical location associated with the subject.

In some embodiments, the filtering further includes determining an availability of each product in the subset of the plurality of nutritional products.

In some embodiments, the recommendation includes one or more descriptions, each description providing information related to an opportunity for an improvement in the health of the subject provided by one or more products in the subset of the plurality of nutritional products, or a reason for inclusion of one or more products in the subset of the plurality of nutritional products.

In some embodiments, the recommendation further includes a dosage regimen for one or more products in the subset of the plurality of nutritional products.

In some embodiments, the recommendation further includes, for each respective nutritional product in the subset of the plurality of nutritional products, one or more products in the plurality of nutritional products as a substitute for the respective nutritional product.

In some embodiments, each decision rule in the plurality of decision rules has the same weight. In some embodiments, one or more decision rules in the plurality of decision rules has a different weight.

Another aspect of the present disclosure provides a method of providing a dietary recommendation to a subject. The method occurs at a computer system. The computer system includes at least one processor and a memory storing at least one program for execution by the at least one processor. The at least one program include instructions for the method. A plurality of assessment responses is obtained in electronic form for an assessment survey presented to the subject. Each respective assessment response, in all or a subset of the plurality of assessment responses, is utilized to select a corresponding set of tags. Each tag in the set of tags is associated with the respective assessment response according to an assessment response to tag lookup data structure. Accordingly, a first plurality of tags is collectively identified. The first plurality of tags is polled against a plurality of decision rules. Each respective decision rule in the plurality of decision rules is independently associated with one or more tags in a second plurality of tags. Moreover, the second plurality of tags includes all the tags in the first plurality of tags. Each time the polling determines that the first plurality of tags includes the one or more tags independently associated with a respective decision rule in the plurality of decision rules, the respective decision rule is fired. This firing of the respective decision rule casts a weighted or unweighted vote associated with the respective decision rule against one or more nutritional products in a plurality of nutritional products specified by the respective decision rule. As such, the firing causes two or more nutritional products in the plurality of nutritional products to have one or more weighted or unweighted votes upon polling all the tags in the first plurality of tags. A subset of the plurality of nutritional products is identified on the basis of the weighted or unweighted votes received by respective nutritional products in the plurality of nutritional products. The subset of the plurality of nutritional products consists of those nutritional products in the plurality of nutritional products that each received a sufficient number of weighted or unweighted votes by the polling to satisfy a nutritional product selection criterion. The subset of nutritional products is filtered against a plurality of periodic nutritional limits. Each respective periodic nutritional limit in the plurality of periodic nutritional limits specifies a corresponding maximum amount of a corresponding dietary supplement that can be consumed in a corresponding period of time given one or more physiological characteristics of the subject specified in the plurality of assessment prompt responses. Further, the filtering independently sums, for each respective dietary supplement associated with each periodic nutritional limit in the plurality of periodic nutritional limits, the corresponding amount of respective dietary supplement in the subset of products. Moreover, the filtering removes one or more doses of one or more nutritional products from the subset of nutritional products when one or more periodic nutritional limits in the plurality of periodic nutritional limits is determined to have been exceeded. This removing one or more doses prevent the one or more periodic nutritional limits from being exceeded. After the filtering, the subset of nutritional products is used to provide a dietary recommendation to the subject.

Yet another aspect of the present disclosure provides a method of providing a dietary recommendation to a subject. The method occurs at a computer system. The computer system includes at least one processor and a memory storing at least one program for execution by the at least one processor. The at least one program include instructions for the method. A plurality of assessment responses is obtained in electronic form for an assessment survey presented to the subject. Each respective assessment response, in all or a subset of the plurality of assessment responses, is utilized to select a corresponding set of tags. Each tag in the set of tags is associated with the respective assessment response according to an assessment response to tag lookup data structure. Accordingly, a first plurality of tags is collectively identified. The first plurality of tags is polled against a plurality of decision rules. Each respective decision rule in the plurality of decision rules is independently associated with one or more tags in a second plurality of tags. Further, at least one tag in the second plurality of tags is incorporated into two or more decision rules in the plurality of decision rules. Moreover, the second plurality of tags includes all of the tags in the first plurality of tags. Each time the polling determines that the first plurality of tags includes the one or more tags independently associated with a respective decision rule in the plurality of decision rules, the respective decision rule is fired. This firing of the respective decision rule casts a weighted or unweighted vote associated with the respective decision rule against one or more nutritional products in a plurality of nutritional products specified by the respective decision rule. As such, the firing causes two or more nutritional products in the plurality of nutritional products to have one or more weighted or unweighted votes upon polling all the tags in the first plurality of tags. A subset of the plurality of nutritional products is identified on the basis of the weighted or unweighted votes received by respective nutritional products in the plurality of nutritional products. The subset of the plurality of nutritional products consists of those nutritional products in the plurality of nutritional products that each received a sufficient number of weighted or unweighted votes by the polling to satisfy a nutritional product selection criterion. The subset of nutritional products is filtered against a plurality of periodic nutritional limits. Each respective periodic nutritional limit in the plurality of periodic nutritional limits specifies a corresponding maximum amount of a corresponding dietary supplement that can be consumed in a corresponding period of time given one or more physiological characteristics of the subject specified in the plurality of assessment responses. Further, the filtering independently sums, for each respective dietary supplement associated with each periodic nutritional limit in the plurality of periodic nutritional limits, the corresponding amount of respective dietary supplement in the subset of products. Moreover, the filtering removes one or more doses of one or more nutritional products from the subset of nutritional products when one or more periodic nutritional limits in the plurality of periodic nutritional limits is determined to have been exceeded. This removing one or more doses prevent the one or more periodic nutritional limits from being exceeded.

Yet another aspect of the present disclosure provides a method of providing a dietary recommendation to a subject. The method occurs at a computer system. The computer system includes at least one processor and a memory storing at least one program for execution by the at least one processor. The at least one program include instructions for the method. A plurality of assessment responses is obtained in electronic form for an assessment survey presented to the subject. Each respective assessment response, in all or a subset of the plurality of assessment responses, is utilized to select a corresponding set of tags. Each tag in the set of tags is associated with the respective assessment response according to an assessment response to tag lookup data structure. Accordingly, a first plurality of tags is collectively identified. The first plurality of tags is polled against a plurality of decision rules. Each respective decision rule in the plurality of decision rules is independently associated with one or more tags in a second plurality of tags. Further, at least one tag in the second plurality of tags is incorporated into two or more decision rules in the plurality of decision rules. Moreover, the second plurality of tags includes all of the tags in the first plurality of tags. Each time the polling determines that the first plurality of tags includes the one or more tags independently associated with a respective decision rule in the plurality of decision rules, the respective decision rule is fired. This firing of the respective decision rule casts a vote associated with the respective decision rule against one or more nutritional products in a plurality of nutritional products specified by the respective decision rule. As such, the firing causes two or more nutritional products in the plurality of nutritional products to have one or more votes upon polling all the tags in the first plurality of tags. A subset of the plurality of nutritional products is identified on the basis of the votes received by respective nutritional products in the plurality of nutritional products. The subset of the plurality of nutritional products consists of those nutritional products in the plurality of nutritional products that each received a sufficient number of votes by the polling to satisfy a nutritional product selection criterion. The subset of nutritional products is filtered against a plurality of periodic nutritional limits. Each respective periodic nutritional limit in the plurality of periodic nutritional limits specifies a corresponding maximum amount of a corresponding dietary supplement that can be consumed in a corresponding period of time given one or more physiological characteristics of the subject specified in the plurality of assessment responses. Further, the filtering independently sums, for each respective dietary supplement associated with each periodic nutritional limit in the plurality of periodic nutritional limits, the corresponding amount of respective dietary supplement in the subset of products. Moreover, the filtering removes one or more doses of one or more nutritional products from the subset of nutritional products when one or more periodic nutritional limits in the plurality of periodic nutritional limits is determined to have been exceeded. This removing one or more doses prevent the one or more periodic nutritional limits from being exceeded. After the filtering, the subset of nutritional products is used to provide a dietary recommendation to the subject.

Yet another aspect of the present disclosure provides a method of providing a dietary recommendation to a subject. The method occurs at a computer system. The computer system includes at least one processor and a memory storing at least one program for execution by the at least one processor. The at least one program include instructions for the method. A plurality of assessment responses is obtained in electronic form for an assessment survey presented to the subject. Each respective assessment response, in all or a subset of the plurality of assessment responses, is utilized to select a corresponding set of tags. Each tag in the set of tags is associated with the respective assessment response according to an assessment response to tag lookup data structure. Accordingly, a first plurality of tags is collectively identified. The first plurality of tags is polled against a plurality of decision rules. Each respective decision rule in the plurality of decision rules is independently associated with one or more tags in a second plurality of tags. Further, at least one tag in the second plurality of tags is incorporated into two or more decision rules in the plurality of decision rules. Moreover, the second plurality of tags includes all of the tags in the first plurality of tags. Each time the polling determines that the first plurality of tags includes the one or more tags independently associated with a respective decision rule in the plurality of decision rules, the respective decision rule is fired. This firing of the respective decision rule casts a weighted or unweighted vote associated with the respective decision rule against one or more nutritional products in a plurality of nutritional products specified by the respective decision rule. As such, the firing causes two or more nutritional products in the plurality of nutritional products to have one or more weighted or unweighted votes upon polling all the tags in the first plurality of tags. A subset of the plurality of nutritional products is identified on the basis of the weighted or unweighted votes received by respective nutritional products in the plurality of nutritional products. The subset of the plurality of nutritional products consists of those nutritional products in the plurality of nutritional products that each received a sufficient number of weighted or unweighted votes by the polling to satisfy a nutritional product selection criterion. Accordingly, the subset of nutritional products is used to provide a dietary recommendation to the subject.

The systems and methods of the present invention have other features and advantages that will be apparent from, or are set forth in more detail in, the accompanying drawings, which are incorporated herein, and the following Detailed Description, which together serve to explain certain principles of exemplary embodiment of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram illustrating an implementation of a system for generating a personalized dietary recommendation, in accordance with an embodiment of the present disclosure, in which optional elements of embodiments are indicated by dashed boxes and/or lines;

FIGS. 2A and 2B collectively illustrate a dietary recommendation server system for generating a dietary recommendation for a user based on inputs provided by the user, in accordance with an embodiment of the present disclosure;

FIG. 3 illustrate a flowchart of a workflow for providing and generating dietary recommendations for a population of users, in accordance with an embodiment of the present disclosure, in which optional elements of embodiments are indicated by dashed boxes and/or lines;

FIG. 4A illustrates a nonlimiting example chart of logical operations that can be used to encode combinations of the presence or absence of specific tags as a precondition for firing a corresponding decisions rule in accordance with an embodiment of the present disclosure;

FIG. 4B illustrates decisions rules, including the logical combination of tags needed to fire the decision rules, in accordance with an embodiment of the present disclosure;

FIG. 4C illustrates a chart for filtering a dietary recommendation according to one or more periodic nutritional limits associated with a nutritional product or a dietary supplement, in accordance with an embodiment of the present disclosure;

FIG. 4D illustrates a chart for an assessment response to tag lookup data structure, in accordance with an embodiment of the present disclosure;

FIGS. 5A and 5B collectively provide a flow chart of methods for generating a dietary recommendation for a user, in accordance with an embodiment of the present disclosure, in which optional elements of embodiments are indicated by dashed boxes and/or lines;

FIGS. 6A, 6B, 6C, and 6D illustrate user interfaces for presenting an assessment for obtaining a plurality of assessment responses, in accordance with an embodiment of the present disclosure;

FIGS. 6E and 6F illustrate user interfaces for presenting a dietary recommendation based on a plurality of assessment responses of an assessment, in accordance with an embodiment of the present disclosure; and

FIGS. 6G and 6H illustrate user interfaces for presenting information about a nutritional product of a dietary recommendation, in accordance with an embodiment of the present disclosure.

Like reference numerals refer to corresponding parts throughout the several views of the drawings.

### DETAILED DESCRIPTION

The present disclosure provides systems and methods for providing a survey to a user, receiving user responses for the survey, and generating a dietary recommendation in response to the user responses. These systems and methods improve the conventional nutrient product systems, offering improved individualized dietary recommendations to each user, and preparing a provision of the dietary recommendation for the user. Moreover, this recommendation provides information related to each nutritional need of the user, each health goal of the user, of nutritional product of the dietary recommendation, or a combination thereof, improving user education for uses and regimens of the nutritional products.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first decision rule could be termed a second decision rule, and, similarly, a second decision rule could be termed a first decision rule, without departing from the scope of the present disclosure. The first decision rule and the second decision rule are both decision rules, but they are not the same decision rule.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

Additionally, the terms “client,” “subject,” and “user” are used interchangeably herein unless expressly stated otherwise.

Furthermore, when a reference number is given an “i^{th}” denotation, the reference number refers to a generic component, set, or embodiment. For instance, a decision rule termed “decision rule i” refers to the i^{th} decision rule in a plurality of decision rules (e.g., a decision rule 122-i in a plurality of decision rules 122).

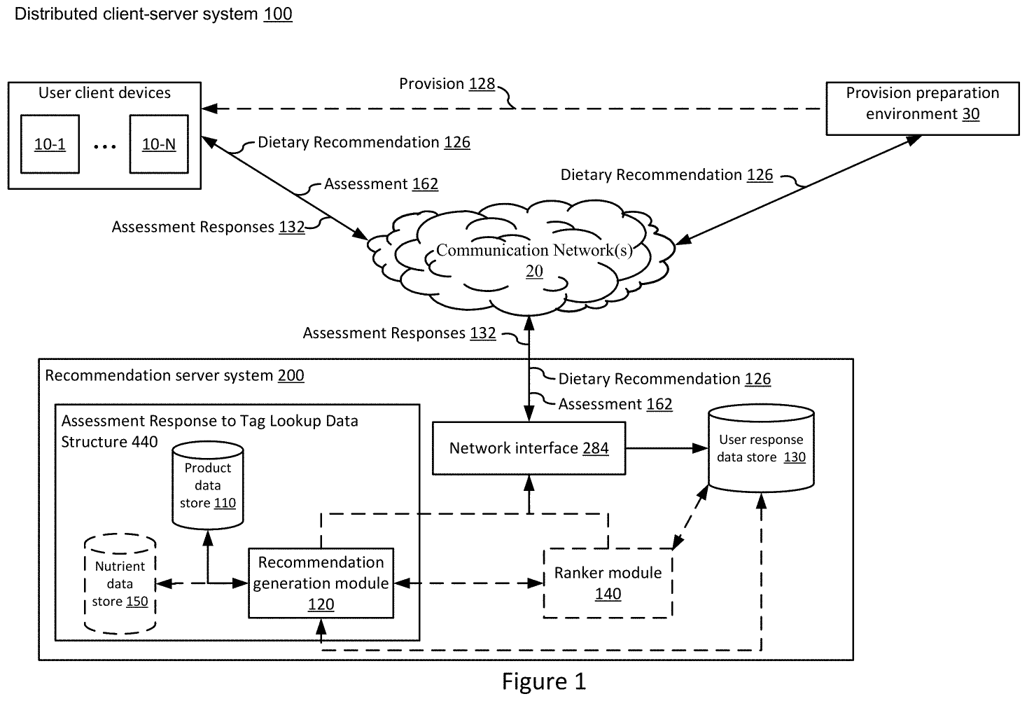

In the present disclosure, unless expressly stated otherwise, descriptions of devices and systems will include implementations of one or more computers. For instance, and for purposes of illustration in FIG. 1, a user device 10-1 is represented as single device that includes all the functionality of the user device 10-1. However, the present disclosure is not limited thereto. For instance, the functionality of the user device 10-1 may be spread across any number of networked computers and/or reside on each of several networked computers and/or by hosted on one or more virtual machines at a remote location accessible across a communications network (e.g., communications network 20). One of skill in the art will appreciate that a wide array of different computer topologies is possible for the user device 10-1, and other devices and systems of the preset disclosure, and that all such topologies are within the scope of the present disclosure.

As used herein, the term “nutritional product” is any commercially available product that is quantifiable (e.g., by mass, volume) that is traditionally consumed by a subject for the purposes of supplementing nutrition or providing all or part of the daily nutritional requirements of a subject. Nutritional products also include intravenous or oral nutrition that can provide all the nutrition. Additional examples of nutritional products, include, but are not limited to iron products, minerals and electrolytes, oral nutritional supplements, vitamin and mineral combinations, and vitamins.

As used herein, the terms “dietary supplement,” “dietary ingredient,” “nutrient,” and “nutritional component” are used interchangeably and refer to individual dietary ingredients within nutritional products as well as particular nutritional products themselves. Examples of dietary supplements include, but are not limited to, minerals, amino acids, herbs, botanicals, as well as other substances that can be used to supplement the diet and that are found in nutritional products. Thus, in some instances, a dietary supplement is an ingredient within nutritional products. In other instances, a dietary supplement is the nutritional product itself, for instance, in cases where the nutritional products consists of a single dietary ingredient.

FIG. 1 depicts a block diagram of a distributed client-server system (e.g., distributed client-server system 100) according to some embodiments of the present disclosure. The system 100 facilitates providing a dietary recommendation assessment (e.g., assessment 162) for a population of users (e.g., user devices 10). The dietary recommendation assessment is provided to each user in the form of a questionnaire, such as a survey. The survey (e.g., an assessment survey, a reassessment survey, etc.) includes a number of prompts that are configured to elicit a response from the user, which guides the system 100 is determining an appropriate dietary recommendation for the user. In response to the survey answers provided by the user (e.g., assessment responses 132), the system 100 provides a recommendation (e.g., dietary recommendation 126) for one or more nutritional products (e.g., nutritional products for a user. Optionally, the system 100 facilitates providing one or more provisions of the one or more nutritional products (e.g., provision 128) of a respective dietary recommendation to the corresponding user.

Of course, other topologies of the system 100 are possible. For instance, in some embodiments, any of the illustrated devices and systems can in fact constitute several computer systems that are linked together in a network, or be a virtual machine or container in a cloud computing environment. Moreover, rather than relying on a physical communications network 20, the illustrated devices and systems may wirelessly transmit information between each other. As such, the exemplary topology shown in FIG. 1 merely serves to describe the features of an embodiment of the present disclosure in a manner that will be readily understood to one of skill in the art.

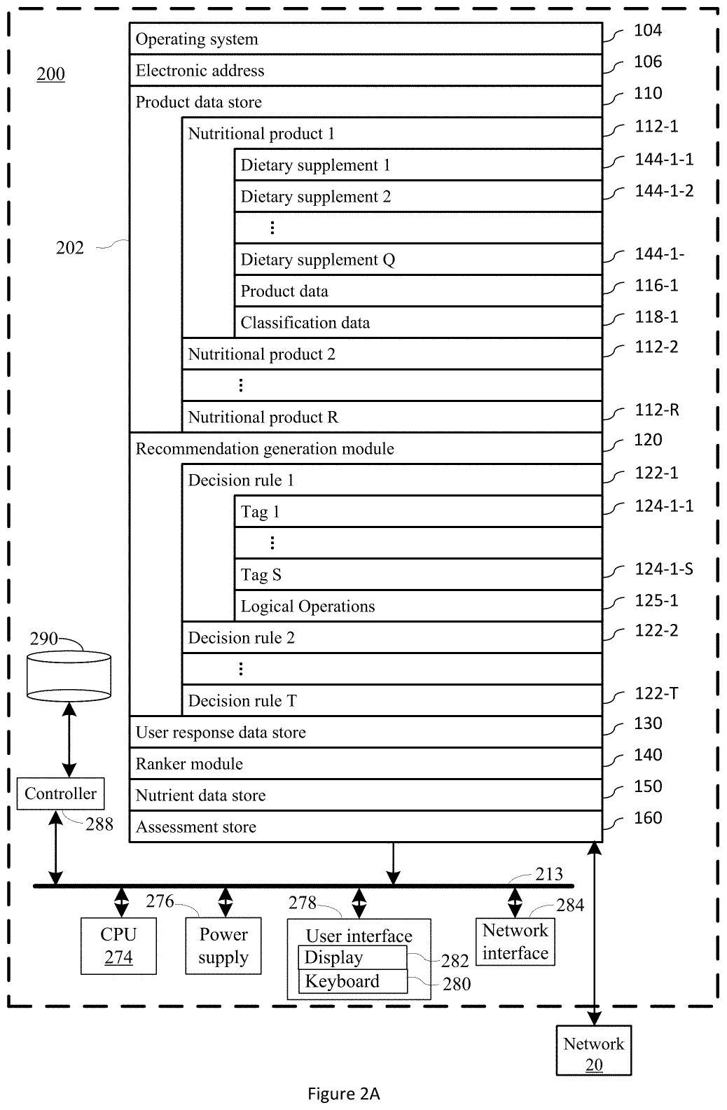

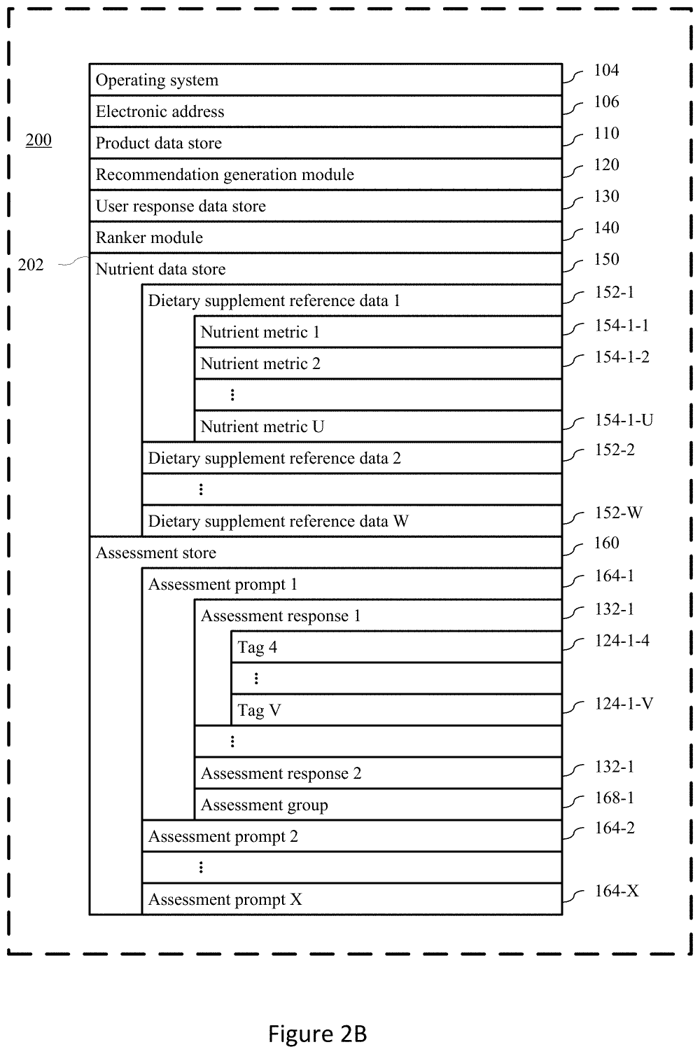

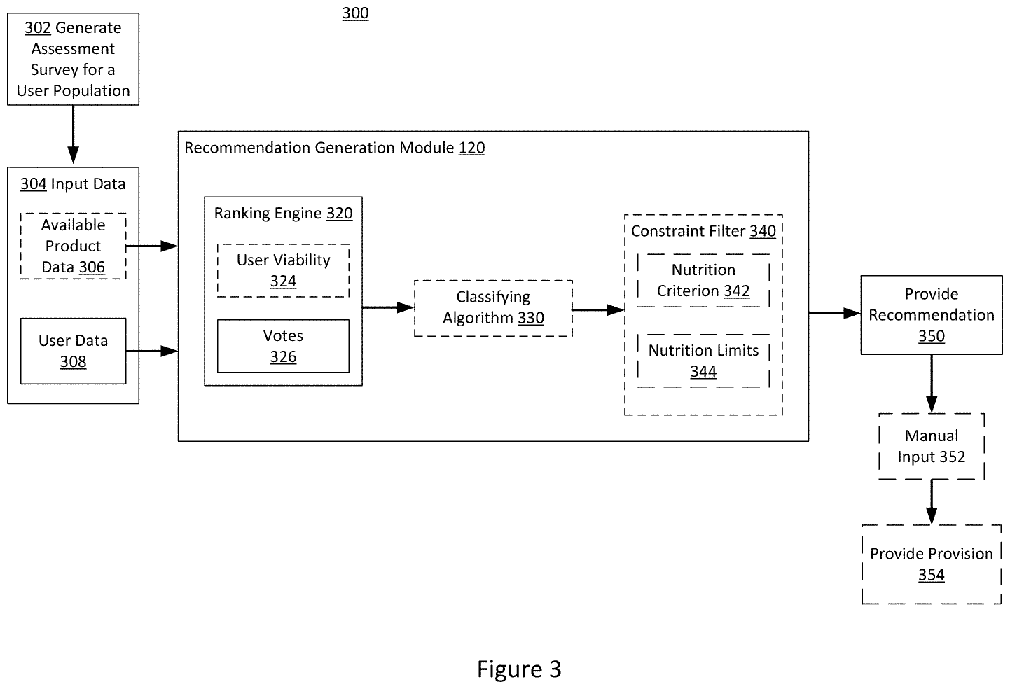

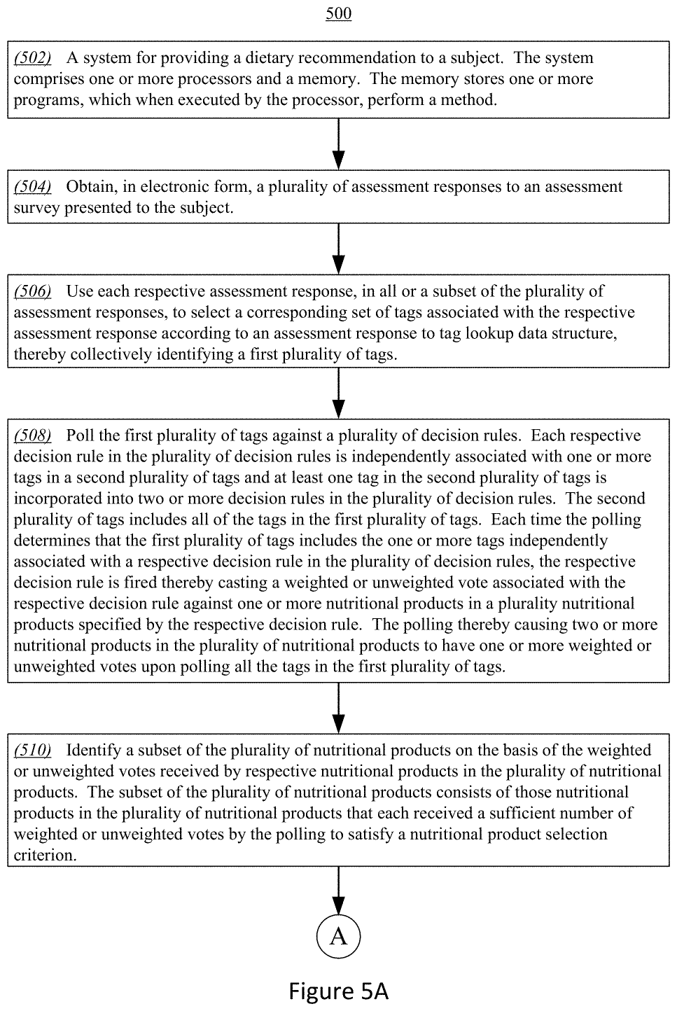

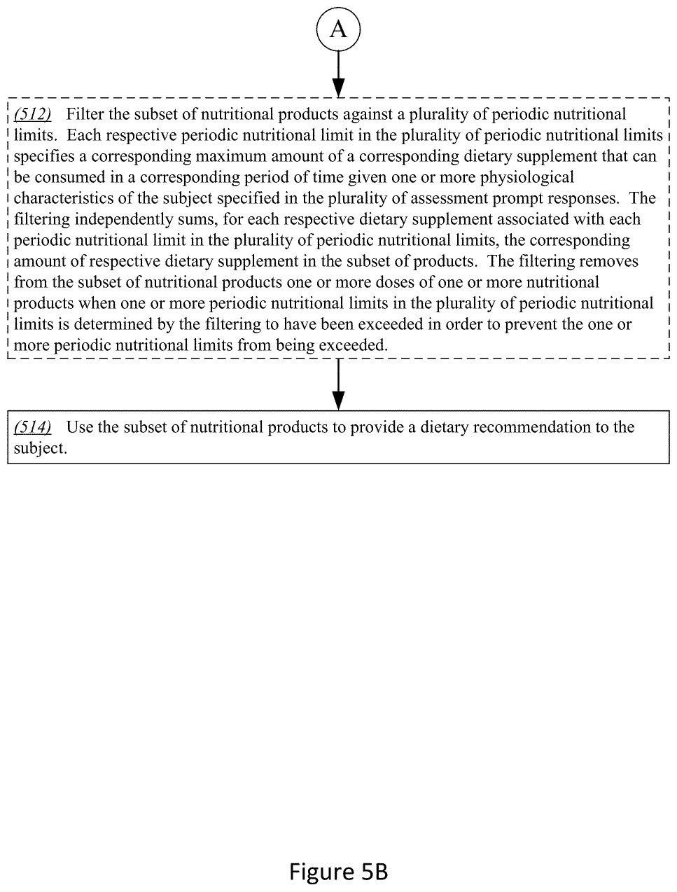

Referring to FIG. 1, in some embodiments, a distributed client-server system 100 includes one or more user devices 10 (e.g., a first user device 10-1, a second user device 10-2, etc.), hereinafter “user device,” each of which is associated with at least one corresponding user (e.g., a consumer of a nutritional product recommendation service). A recommendation server system 200 provides an assessment survey (e.g., assessment survey 162 of FIG. 1; generation of assessment survey 302 of FIG. 3) to the one or more user devices 10 for generating a dietary recommendation (e.g., dietary recommendation 126 of FIG. 1; providing recommendation 350 of FIG. 3). The user devices 10 return one or more assessment responses (e.g., assessment responses 132 of FIG. 1; user data 308 of FIG. 3; block 504 of FIG. 5A) based on various assessment prompts (e.g., assessment prompt 164 of FIG. 2B) of the assessment survey 162. The one or more responses 132 are associated with the corresponding user that provided the one or more assessment responses 132 responsive to the assessment survey 162. After evaluating the provided assessment responses 132 (e.g., recommendation generation module 120 of FIG. 3; blocks 506 through 512 of FIG. 5A and FIG. 5B), the recommendation system 200 provides a dietary recommendation 216 to the corresponding user (e.g., block 514 of FIG. 5B). Moreover, in some embodiments, the client system 100 includes one or more provision preparation environments 30 to prepare various provisions 128 of one or more nutritional products (e.g., nutritional products 112 of FIG. 2A) from a dietary recommendation 126 for a respective user.

In some embodiments, a user device 10 includes a mobile device, such as a mobile phone, a tablet, a laptop computer, a wearable device such as a smart watch, and the like. Alternatively, the user devices 10 may be a desktop computer or other similar devices. Further, in some embodiments, the user devices 10 (e.g., user device 10-1, user device 10-2, user device 10-3, . . . , user device 10-N) communicate with a centralized user device (e.g., a server) that facilitates allocating the assessment 162 to each respective user device 10. Moreover, this centralized user device may receive and/or combine each assessment response 132 received by each user device 10 in order to communicate a single collective assessment response 132. Further, in some embodiments, each user device 10 enables a respective user to provide information related to the respective user (e.g., user preferences, dietary needs, etc.). Each user device 10 includes one or more processors, a memory coupled to the one or more processors, a display, and means to input commands such a touchscreen, a keyboard, a mouse, a microphone, or a similar device to detect or sense an input provided by the corresponding user.

In some embodiments, the communication network 20 optionally includes the Internet, one or more local area networks (LANs), one or more wide area networks (WANs), other types of networks, or a combination of such networks.

Examples of communication networks 20 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSDPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

As illustrated in FIG. 1, in some embodiments, the recommendation system 200 includes a recommendation generation module (e.g., recommendation generation module 120 of FIG. 2A; recommendation generation module 120 of FIG. 3) that facilitates generating various nutritional product recommendations (e.g., dietary recommendation 126 of FIG. 1) for a population of users (e.g., user devices 10).

In some embodiments, the recommendation system 200 generates a dietary recommendation 126 using information provided by the various devices and modules of the present disclosure (e.g., survey responses 132 of FIG. 1 provided by a user device 10-1, user responses of the user response data store 130 of FIG. 2A, etc.). In some embodiments, the recommendation system 200 provides a unique dietary recommendation 126 for each respective user, such that no two users are provided with identical dietary recommendations 126. In some embodiments, the recommendation system 200 provides an independent dietary recommendation 126 for each respective user, but it is possible that some users coincidently receive identical dietary recommendations 126. In some embodiments, the unique dietary recommendation 126 includes an independent selection of one or more nutritional products 112 for each respective user, an independent reasoning (e.g., motivation) for including one or more nutritional products 112 of the dietary recommendation 126 (e.g., a first user 10-1 is recommended a first nutritional product 112-1 for improved bone health and a second user 10-2 is recommended the first nutritional product 112-1 for improved joint function), or a combination thereof. Furthermore, in some embodiments, the recommendation system 200 provides a dietary recommendation 126 selected from a variety of predetermined dietary recommendation 126, such as predetermined classifications (e.g., classification 118 of FIG. 2A) of one or more nutritional products 112.

In some embodiments, the recommendation generation module 120 exchanges data with other modules (e.g., a product data store 110, a user response data store 130, a ranker module 140, a nutrient data store 150, or a combination thereof) of the recommendation system 200 in order to incorporate various data received therefrom in the generation of a dietary recommendation (e.g., input user response data 132 of FIG. 2B). For instance, in some embodiments, the recommendation generation module 120 communicates with the product data store 110 (e.g., product data store 110 of FIG. 2A; product availability 306 of FIG. 3) and forms a number of determinations to generate the dietary recommendation 126 based on this communicated information.

In some embodiments, the recommendation generation module 120 accesses the product data store 110 to incorporate various nutritional product 112 data (e.g., product data 116 of FIG. 2A) in forming a determination of an appropriate dietary recommendation 126 for a respective user. For instance, in some embodiments, the product data 116 includes data regarding an availability of one or more nutritional products 112 (e.g., available product data 306 of FIG. 3), such as a variety of stock keeping units (SKUs) describing an inventory associated with the nutritional products 112 (e.g., inventory of a nutritional product 112 of the provision preparation environment 30 of FIG. 1). In some embodiments, one or more nutritional products 112 have a limited availability (e.g., a season product, a geographic restriction, a limited shelf life, etc.), which is considered in forming the determination of the appropriate dietary recommendation 126.

In some embodiments, the recommendation generation module 120 classifies (e.g., classifying algorithm 330 of FIG. 3) available nutritional products 112 based on their respective nutritional components (e.g., dietary supplements 114 of FIG. 2A). In some embodiments, this classifying utilizes a classifying algorithm 330, e.g., identifying groups of nutritional products 112 with significant overlap in their nutritional components (i.e., dietary supplements 114), or similar health benefits, and forming one or more classifications 118 of the overlapping nutritional products 112. In this fashion, a respective dietary recommendation 126 is identified through a common dietary goal (e.g., a first classification 118-1 associated with a goal to increase strength, a second classification 118-2 associated a goal for to reduce sugar intake, etc.) and/or common nutritional product 112 recommendations (e.g., a first classification 118-1 associated with prenatal users, a second classification 118-2 associated a postnatal users, etc.).

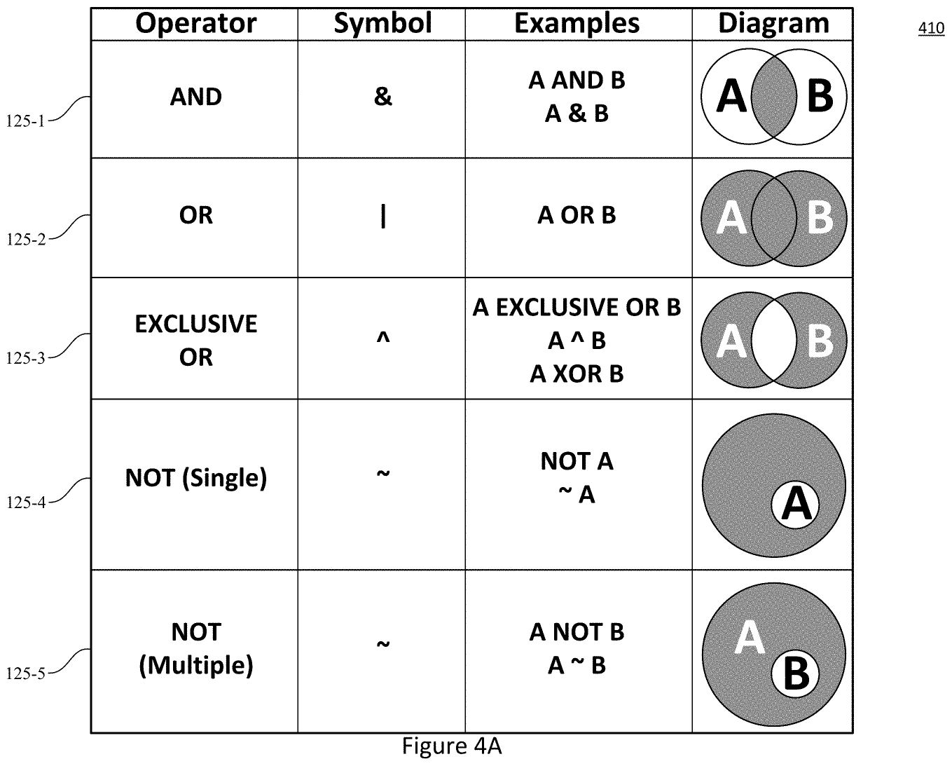

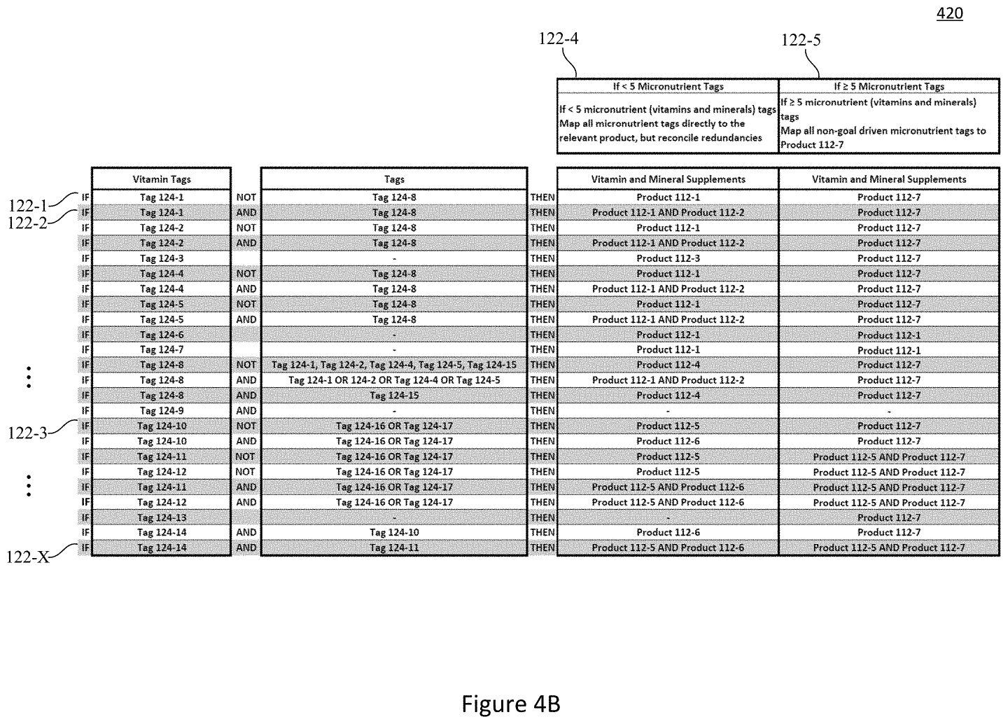

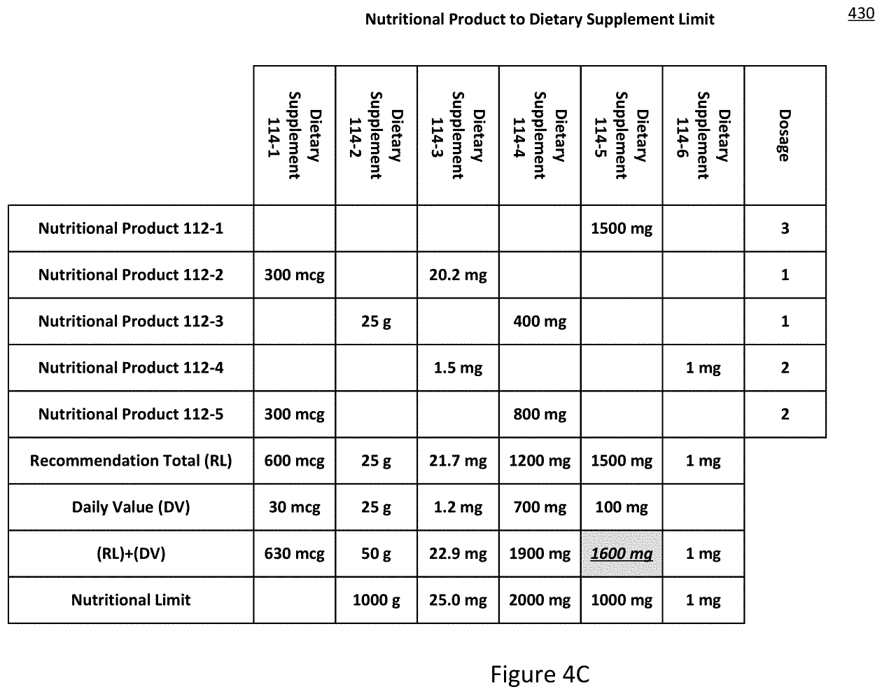

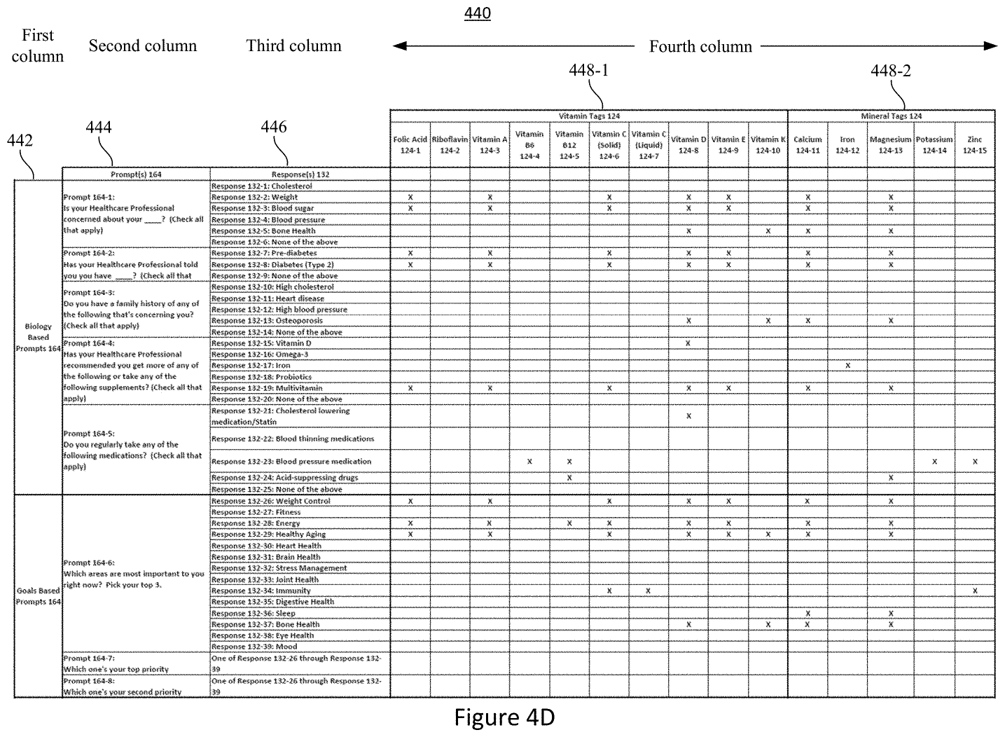

In some embodiments, the recommendation generation module 120 applies one or more filters (e.g., constraint filter 340 of FIG. 3, block 512 of FIG. 5B) that further refine the dietary recommendation 126. However, the present disclosure is not limited thereto. For instance, in some embodiments, the filtering ensures a respective dietary recommendation 126 excludes one or more redundant (e.g., excessive) dietary supplement 114 (e.g., nutrition criterion 343 of FIG. 3), including preventing dietary supplement 114 stacking such as a first vitamin C supplement and a second vitamin C supplement in a single dietary recommendation that in combination exceed a recommendation dosage of vitamin C (e.g., chart 430 of FIG. 4C).

Similarly, in some embodiments, the filtering ensures that one or more choices of nutritional products 112 available to a user through a dietary recommendation 126 satisfies the conditions of the dietary recommendation in view of one or more user preferences (e.g., a user preference for fluid based nutritional products 112) and/or one or more dietary restrictions indicated by the corresponding user (e.g. filtering one or more nutritional products 112 including lactose if an assessment response 132 indicates the corresponding user is lactose intolerant; user viability 324 of FIG. 3), to ensure that the respective dietary recommendation 126 achieves a desired health goal of the corresponding user.

In some embodiments, the dietary recommendation 126 ensures that a minimum number of nutritional product 112 choices are available for each type of nutritional product 112 offered through the dietary recommendation 126 (e.g., manual input 352 of FIG. 3), such as offering a minimum of three protein source nutritional products 112 for a user to select from. This minimum number of nutritional product 112 choices allows each user to tailor a provision 128 of the dietary recommendation 126 to the preferences of the user while still satisfying various requirements of the dietary recommendation 126.

In some embodiments, the recommendation generation module 120 interfaces with a user response data store 130 to retrieve information about a population of users or a specific user that a dietary recommendation 126 is being generated for. In some embodiments, the information retrieved from the user response data store 130 includes one or more user preferences (e.g., a first user preference for whey protein sources instead of soy protein sources, a second user preference for a solid dosage form, etc.), a listing of previously obtained assessment responses 132 for one or more users, a listing of previously obtained provisions 128 and/or nutritional product 112 selections for one or more users (e.g., previously obtained manual input 352 of FIG. 3), one or more user experience constraints (e.g., promoting a particular nutritional product 112; providing a more diverse selection of nutritional products 112 to the user in a dietary recommendation 126), or a combination thereof. This information allows the recommendation generation module 120 to consider a pallet and uniqueness of each respective user and/or user population, which provides a dietary recommendation 126 tailored to the user, as well as providing an enjoyable experience for each user, such as reduced cognitive burden for the user to select nutritional products 112. In some embodiments, this information allows the recommendation generation module 120 to formulate one or more predictive measures of a user assessment response 132, such as a prediction of a user selection of a first nutritional product 112-2 based on previous user selections for one or more provisions 128 having the first product 112. This dietary recommendation 126 also prevents each user from selecting nutritional products 112 that will influence their health negatively (e.g., nutritional limits 344 of FIG. 3), such as exceeding a recommendation dosage (e.g., satisfying a nutrient metric 154 of FIG. 2B) for a respective dietary supplement 114.

In some embodiments, the recommendation server system 200 applies previous user response data 130 to a machine learning algorithm that forms various predictions related to user preferences, predicted user assessment responses 132, predicted nutritional product 112 selections, and the like.

In some embodiments, the algorithm includes a linear regression algorithm. Linear regression algorithms are disclosed in James, Witten, Hastie, and Tibshirani, An Introduction to Statistical Learning, 2013, Springer Science+Business Media New York, which is hereby incorporated by reference.

In some embodiments, the algorithm includes logistic regression algorithm. Logistic regression algorithms are disclosed in Agresti, An Introduction to Categorical Data Analysis, 1996, Chapter 5, pp. 103-144, John Wiley & Son, New York, N.Y., which is hereby incorporated by reference.

In some embodiments, the algorithm includes a linear discriminant analysis algorithm. Linear discriminant analysis algorithms are is disclosed in Izenman, 2013, “Linear Discriminant Analysis,” In: Modern Multivariate Statistical Techniques, Springer Texts in Statistics. Springer, New York, N.Y., which is hereby incorporated by reference.

In some embodiments, the algorithm includes a decision tree algorithm such as a classification and regression tree (CART) algorithm. Other specific suitable decision tree algorithms include, but are not limited to Random Forest, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forest algorithms are described in Breiman, 1999, “Random Forests—Random Features,” Technical Report 567, Statistics Department, U.C. Berkeley, September 1999, which is hereby incorporated by reference in its entirety.

In some embodiments, the algorithm includes a naïve Bayes algorithm. Naïve Bayes algorithms are disclosed in Rosen et al. 2001, Bioinformatics 27(1):127-129, which is hereby incorporated by reference.

In some embodiments, the algorithm includes a K-Nearest neighbor algorithm. K-Nearest neighbor algorithms are disclosed in Kamvar et al., 2015, Front Genetics 6:208 doi: 10.3389/fgene.2015.00208), which is hereby incorporated by reference.

In some embodiments, the algorithm includes an artificial neural network (e.g., learning vector quantization) algorithm. Artificial neural network algorithms are disclosed in Hassoun, 1995, Fundamentals of Artificial Neural Networks, Massachusetts Institute of Technology, which is hereby incorporated by reference.

In some embodiments, the algorithm includes a support vector machine algorithm. Support vector machine algorithms are disclosed in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety.

In some embodiments, the algorithm includes a boosting algorithm. Boosting algorithms are disclosed in Schapire and Freund, 2013, “Boosting: Foundations and Algorithms,” Kybernetes 42(1), pp. 164-166, which is hereby incorporated by reference in its entirety.

In some embodiments, algorithm includes any combination of the pattern classification and/or regression algorithms disclosed herein.

In some embodiments, generating the dietary recommendation 126 (e.g., providing recommendation 350 of FIG. 3) includes using one or more algorithms, such as a classifying algorithm (e.g., classifying algorithm 330 of FIG. 3). In some embodiments, the classifying algorithm 330 aggregates one or more nutritional products 112 into a respective classification 118 of nutritional products 112, or various sub-groupings of one or more nutritional products 112. In some embodiments, the classification 118 of one or more nutritional products 112 is selected as a predetermined dietary recommendation 126 to be included in a generated dietary recommendation 126 for a respective user population or a respective user (e.g., all prenatal, obese, vegan women of 25 years of age or younger are provided with a first dietary recommendation 126-1 that is classified 118-1 for prenatal obese vegan women of 25 years of age or younger). For instance, in some embodiments, a variety of classifications 118 of nutritional products 112 is formed with one or more nutritional products 112 overlapping between respective classifications 118, similar to the overlapping of a Venn-diagram (e.g., both of a first classification 118-1 and a second classification 118-2 include a first nutritional product 112-1). This overlapping, classifying selection may include comparing a variety of commonalities between various nutritional products 112, user populations, user responses 132, or a combination thereof. In some embodiments, the classifying algorithm includes applying a variety of filters to the classified recommendations 126 available to a user population to generate a dietary recommendation 126 (e.g., filtering to remove one or more classifications 118 that include vegan nutritional products 112 and one or more classifications 118 that excludes dairy based nutritional products 112 for a user population of lactose intolerant vegetarians). For instance, in some embodiments, one or more filters is used to refine a database of nutritional products 112 (e.g., product data store 110) into an appropriate number of nutritional product 112 for a dietary recommendation 126.

In the present disclosure, the provision preparation environment 30 is a physical environment (e.g., a retail store, a warehouse, a factory production facility, etc.) that is tasked with preparing a provision 128 of nutritional products 112 from a dietary recommendation 126 for a user. In the present disclosure, the provision preparation environment 30 will be described as an all-encompassing general environment that is configured to facilitate the preparation (e.g., storing, preparing, processing, handling, etc.) and whole production (e.g., packaging, delivery, etc.) of various provisions 128. However, the present disclosure is not limited thereto. For instance, in some embodiments, the provision preparation environment 30 is an industrial facility, including a fully, or partially, automated factory system, a human operated system, or a combination thereof. Moreover, in some embodiments, the provision preparation environment 30 includes a service provider that is contracted for work (e.g., a co-packer).

Once a dietary recommendation 126 is generated (e.g., via the recommendation generation module 120), the dietary recommendation 126 is communicated to a corresponding user device 10. In some embodiments, the corresponding user device 10 that receives the respective dietary recommendation 126 is the same user device 10 that provided user responses 132 for generating the respective dietary recommendation 126, or likewise a different user device 10. In some embodiments, the corresponding user device 10 receives the dietary recommendations 126 (e.g., a personalized dietary recommendation 126 based on input user data 304 of FIG. 3 such as the dietary profile of the user, e.g., stored in user selection data store 130) via communication network 20.

In some embodiments, each dietary recommendation 126 provides a respective user with one or more choices of nutritional products 112 to receive in a provision 128 of the dietary recommendation 126, allowing the user to customize and modify the provision 128 to their specific needs (e.g., manual input 352 of FIG. 3). As such, the user device 10 communicates the corresponding user choices (e.g., an order for a provision 128) to the recommendation server system 200 via the communication network 20. In some embodiments, the user choices include substituting one or more nutritional products 112 in the dietary recommendations 126 (e.g., changing a protein source selection from a whey protein source nutritional product 112-1 to a plant protein source nutritional product 112-3), modifying a portion size of a nutritional product 112 the dietary recommendation 126 (e.g., changing a nutritional product 112 selection from a first dosage of the nutritional product 112 to a second dosage, such as a modification in the dosages of FIG. 4C), eliminating a respective nutritional product 112 from the dietary recommendation 126 (e.g., omitting one or more nutritional products 112 that include a gelatin (e.g., non-vegan) capsule), and the like. In some embodiments, these provision 128 choices communicated from the user devices 10 are stored in the user response data store 130, allowing the system 100 to provide improved future dietary recommendations 126 for each respective user guided by previous choices of the user.

Now that a distributed client-server system 100 has generally been described, an exemplary recommendation server system 200 for generating a personalized dietary recommendation 126 for a user will be described with reference to FIG. 1 through FIG. 2B. In various embodiments, the recommendation server system 200 includes one or more processing units (CPUs) 274, a network or other communications interface 284, a memory 202 (e.g., a random access memory), one or more magnetic disk storage and/or persistent device 290 optionally accessed by one or more controllers 288, one or more communication busses 213 for interconnecting the aforementioned components, and a power supply 276 for powering the aforementioned components. In some embodiments, data in memory 202 is seamlessly shared with non-volatile memory 290 using known computing techniques such as caching. In some embodiments, memory 202 and/or memory 290 may in fact be hosted on computers that are external to the recommendation system 200 but that can be electronically accessed by the recommendation system 200 over an Internet, intranet, or other form of network or electronic cable (e.g., element 20 in FIG. 2A) using network interface 284.

In some embodiments, the memory 202 of the recommendation system 200 for generating a personalized dietary recommendation 126 stores:
an operating system 104 (e.g., ANDROID, iOS, DARWIN, RTXC, LINUX, UNIX, OS X, WINDOWS, or an embedded operating system such as VxWorks) that includes procedures for handling various basic system services;
an electronic address 106 associated with the recommendation system 200 that identifies the recommendation system 200;
a product data store 110 for storing a variety of data related to one or more nutritional products 112, each nutritional product 112 including one or more dietary supplements 114 (e.g., a first nutritional product 112-1 includes a first dietary supplement 114-1 of a protein source and a second dietary supplement 114-2 of iron), a variety of product data 116 describing one or more metrics of the nutritional product 112, and a variety of classification data describing one or more classifications 118 associated with the respective nutritional product 112;
a recommendation generation module 120 (e.g., recommendation generation module 120 of FIG. 3) that facilitates generating a variety of dietary recommendations 126 for a population of users based on one or more decision rules 122;
a user response data store 130 that stores one or more assessment responses 132 provided by respective users in the population of users and, optionally, previous assessment responses 132 made by the respective users in the population of users and/or dietary information about the respective users in the population of users (e.g., physiological characteristics of a respective user such as a diet type, a dietary restriction, a dietary preference, a dietary or health goal, and the like);
a ranker module 140 (e.g., ranking engine 320 of FIG. 3) that facilitates analyzing and mapping one or more nutritional product 112 selection criterion and assessment responses 132 to determine a respective dietary recommendation 126 (e.g., block 510 of FIG. 5A);
a nutrient data store 150 that stores a variety of dietary supplement reference data 152, each respective dietary supplement reference data 152 having one or more nutrient metrics 154 related to a feature of the respective dietary supplement reference data 152, such as a maximum dosage of a corresponding dietary supplement 114 associated with the respective dietary supplement reference data 152, (e.g., nutritional limit of FIG. 4C); and
an assessment store 160 that stores, and optionally generates one or more assessment surveys 162 (e.g., generate assessment survey 302 of FIG. 3) including one or more assessment prompts 164 that are provided to a user device 10 to elicit an assessment response 132 for determining a personalized dietary recommendation 126 for the corresponding user of the user device 10.

An electronic address 106 is associated with the recommendation system 200, which is utilized to at least uniquely identify the recommendation system 200 from other devices and components of the distributed system 100. In some embodiments, the electronic address 106 associated with the recommendation system 200 is used to determine the source of a communication received from (e.g., communicating assessment survey 162 and/or dietary recommendation 126 to one or more user devices 10) and/or provided to the recommendation system 200 (e.g., receiving assessment responses 132 from one or more user devices 10).

A product data store 110 stores a variety of data related to one or more nutritional products 112, which is potentially included in a dietary recommendation 126. Each nutritional product 112 is a consumable good (e.g., a food product such as a solid or a liquid food; a powder product such as a protein powder, a dosage form such as a capsule or a tablet, etc.) that provides a beneficial effect to the heath of the user (e.g., a physiological function to maintain health). In some embodiments, a respective nutritional product 112 includes a combination of two or more goods, such as a first nutritional product 112-1 including a first portion configured for consumption prior to a user undertaking an activity (e.g., a pre-workout portion of first nutritional product 112-1) and a second post-activity portion (e.g., a post-workout portion of the first nutritional product 112-1), or the like. In some embodiments, the combination of two or more goods for a respective nutritional product 112 include a solvent portion (e.g., an electrolyte fluid solvent) and a solute portion (e.g., a granulated prebiotic formulation solute) that in combination form a solution of the first nutritional product 112.

Each nutritional product 112 includes one or more dietary supplements 114 that describe one or more nutritional aspects (e.g., components) of the corresponding nutritional product 112. In some embodiments, the one or more of the dietary supplements 114 associated with a respective nutritional product 112 describes a macronutrient of the respective nutritional product 112 (e.g., a fat, a protein, or a carbohydrate in the respective nutritional product 112). In some embodiments, the one or more of the dietary supplements 114 associated with a respective nutritional product 112 describes a micronutrient of the respective nutritional product 112 (e.g., a respective vitamin in the nutritional product 112, a respective mineral in the nutritional product 112, etc.). For instance, if a first nutritional product 112-1 is a protein powder product having zero mass (e.g., grams (g)) of carbohydrates, zero mass of fat, and a mass, which is greater than zero, of protein per serving (e.g., dosage) of the nutritional product 112-1, the product data store 110 for the first nutritional product 112-1 will include at least a first dietary supplement 114-1-1 associated with protein for the first product 112-1.

In some embodiments, one or more dietary supplements 114 associated with a respective nutritional product 112 describes a micronutrient of the respective nutritional product 112, such as a vitamin dietary supplement, a mineral dietary supplement, a functional dietary supplement, or a specialty dietary supplement (e.g., an herbal dietary supplement, a botanical dietary supplement, etc.). For instance, in some embodiments, a respective dietary supplement 114 is associated with a vitamin including vitamin A, riboflavin (e.g., vitamin B2), vitamin B6, folic acid (e.g., folate or vitamin B9), vitamin B12, vitamin C, vitamin D, vitamin E, vitamin K, or a combination thereof (e.g., a first dietary supplement 114-1 associated with vitamin A, a second dietary supplement 114-2 associated vitamin B2, etc.). In some embodiments, the vitamin that a respective dietary supplement 114 is associated with further includes thiamin (e.g., vitamin B1), niacin (e.g., vitamin B3), pantothenic acid (e.g., vitamin B5), biotin (e.g., vitamin H or vitamin B7), or a combination thereof.

In some embodiments, a respective dietary supplement 114 is a mineral such as calcium, iodine, iron, magnesium, potassium, zinc, or a combination thereof. In some embodiments, the respective mineral dietary supplement 114 is chromium, copper, manganese, molybdenum, phosphorus, selenium, sodium, or a combination thereof. Furthermore, in some embodiments, a respective mineral dietary supplement 114 comprises boron, nickel, silicon, tin, vanadium, or a combination thereof (e.g., associated with a trace mineral).

In some embodiments, a respective dietary supplement 114 is a specialty dietary supplement such as anise, ashwaganda, black cohosh, boswellia, one or more carotenoids, chromium, one or more digestive enzymes, one or more flavonoids (e.g., one or more catechins), glucosamine, ginger, glucosamine, gamma lionlenic acid (GLA), guarana, isoflavones, L-Theanine, L-Tyrosine, nitrate, omega-3 fatty acid (e.g., a fish oil), peppermint, one or more polyphenols, one or more biotics (e.g., one or more probiotics), one or more plant sterols/stanols, one or more procyanidins, ubiquinone (e.g., coenzyme Q-10), quercetin, resveratrol, saw palmetto, senna glycoside (e.g., sennoside or senna), turmeric, tart cherry extract, valerian, Saint John's wort, or a combination thereof.

In some embodiments, a respective dietary supplement 114 is a functional dietary supplement such as caffeine, one or more electrolytes, fiber, one or more greens, menthol, one or more proteins (e.g., a performance whey protein, a soy protein, a recovery protein, etc.), one or more biotics (e.g., one or more prebiotics), or a combination thereof. Furthermore, in some embodiments, a respective dietary supplement 114 comprises a hormone (e.g., an over-the-counter (OTC) hormone) or an amino acid.

In some embodiments, a respective dietary supplement 114 is subgroup of a corresponding broader dietary supplement 114. For instance, in some embodiments, a respective dietary supplement 114 includes, or is divisible into, two or more subgroups of dietary supplements 114 (e.g., a broader caffeine 114 dietary supplement includes a first caffeine formulation 114-1 and a second caffeine formulation 114-2). In some embodiments, the two or more subgroups represent various dosages forms of a respective dietary supplement 114. In some embodiments, the two or more subgroups represent various dosages of the respective dietary supplement 114, such as a first omega-3 dietary supplement 114-1 representing a first dosage (e.g., 1000 milligrams (mg) of omega-3) and a second omega-3 dietary supplement 114-2 representing a second dosage (e.g., 2,000 mg of omega-3). In some embodiments, the two or more subgroups represent various forms of the respective dietary supplement 114, such as a first protein dietary supplement 114-1 representing a whey protein source and a second protein dietary supplement 114-2 representing a plant based protein source. In some embodiments, the two or more subgroups represent different additives in combination with the respective dietary supplement 114, such as a first caffeine dietary supplement 114-1 of caffeine with a first additive (e.g., a sugar-free caffeine dietary supplement) and a second caffeine dietary supplement 114-2 of a caffeine with a second additive (e.g., a caffeine dietary supplement including sugar).

In some embodiments, the product data store 110 further includes a variety of product data 116 that is associated with the nutritional products 112. In some embodiments, for each respective dietary supplement 114 of a corresponding nutritional product 112, the product data 116 includes various data describing a quantity of each respective dietary supplement 114 included within the nutritional product 112. In some embodiments, this quantity of the respective dietary supplement 114 includes a mass of the dietary supplement 114 per unit mass of the nutritional product 112, such as a number of milligrams (mg) of the respective dietary supplement 114 per gram of the nutritional product 112, a mass of the respective dietary supplement 114 per serving of the nutritional product 112, and the like. In some embodiments, the data describing a quantity of the respective dietary supplement 114 of the nutritional product 112 is derived from a nutrition facts label (e.g., a United States Food and Drug Administration food label) associated with the nutritional product 112.

In some embodiments, the product data 116 for a respective nutritional product 112 includes one or more constraints associated with the nutritional product 112 that are evaluated in determining a personalized dietary recommendation 126 for a respective user. In some embodiments, the constraints stored in the product data 116 include a drug interaction associated with the respective nutritional product 112, such as a pharmacokinetic drug interaction and/or a pharmacodynamics drug interaction. Generally, a pharmacokinetic drug interaction is an interaction between two compounds (e.g., a first dietary supplement 114-1 and a second dietary supplement 114-2) that result in alterations in the absorption, transport, distribution, metabolism, and/or excretion of either dietary supplement. Generally, a pharmacokinetic drug interaction is an interaction between two compounds (e.g., a first dietary supplement 114-1 and a third dietary supplement 114-3) that result in a direct change in the effect of either compound. For a more comprehensive summary of pharmacokinetic drug interactions and pharmacodynamics drug interactions, see, Cascorbi, I, Dtsch Arztebl Int., 109(33-34):546-55 (2012), the content of which is hereby incorporated by reference. Since users more often than not fail to discuss supplemental user of nutritional products 112 with a medical practitioner, it is important for the recommendation generation model 120 to consider the above described drug interaction constraints in generating a dietary recommendation 126 for the users. Bailey et al., 2013, “Why US Adults Use Dietary Supplements,” JAMA Internal Medicine, 173(5), pg. 355.

In some embodiments, the one or more constraints of the product data 116 associated with a respective nutritional product 112 includes an indication to consume the nutritional product 112 during a specified metabolic state, such as on an empty stomach, with a meal, after consuming a meal, with fluids, and the like. In some embodiments, the one or more constraints of the product data 116 associated with a respective nutritional product 112 includes an indication to consume the nutritional product 112 during a specified period of time (e.g., within a first period of time before falling asleep, within a second period of time after waking up, etc.), and the like. In some embodiments, the recommendation generation module 120 recommends one or more nutritional products 112 that have the same constraints, such that the user is provided the opportunity to consume a provision 128 of the dietary recommendation (e.g., a provision 128 configured for quaque die consumption).

In some embodiments, the one or more constraints of the product data 116 associated with a respective nutritional product 112 includes one or more dimensional constraints associated with the nutritional product 112. In some embodiments, the dimensional constraint includes a physical dimensional constraint of the nutritional product 112 that defines a tangible constraint of the nutritional product 112. For instance, in some embodiments, the physical dimensional constraint for a respective nutritional product 112 includes a physical size of the nutritional product 112 (e.g., a first product 112-1 is ginger with a volume of one cubic centimeter (cm³) per dosage of the first product 112-1, a second product 112-2 is a meal supplement bar with a volume of 120 cm³ per dosage of the second product 112-2, etc.). Accordingly, in some embodiments, a respective provision 128 of a dietary recommendation 126 includes a maximum dimensional size, such that each respective nutritional product 112 included in the provision 128 must collectively satisfy the maximum dimensional size of the provision. In some embodiments, the maximum dimensional size for a respective nutritional product 112 includes a size of a dosage of the provision 128. In some embodiments, the maximum dimensional size is determined by a shipping container for providing the provision 128 to a respective user, such that the provision 128 includes a limited volume that each nutritional product 112 must collectively satisfy (e.g., provide provision 354 of FIG. 3).

In some embodiments, the dimensional constraint includes a partition constraint of the nutritional product 112. The partition constraint describes an ability to partition (e.g., sub-divide) the respective nutritional product 112 into one or more provisions 128, or dosages, of the nutritional product 112. In some embodiments, the partition constraint of a respective nutritional product 112 includes one or more available dosages of the respective nutritional product 112 (e.g., a first product 112-1 is commercially available in a first dosage, a second dosage, and a third dosage). In some embodiments, the partition constraint for the respective nutritional product 112 includes a number of dosage forms of the respective nutritional product 112 (e.g., an available inventory of dosages), such that a dietary recommendation 126 can modify an amount of the respective nutritional product 112 within a provision 128 of the dietary recommendation 126 by increasing, or similarly decreasing, the number of dosage forms of the respective nutritional product 112 within the provision 128. For instance, consider a first nutritional product 112-1 provided in a tablet dosage form that is commercially available as a 10 mg tablet dosage form or a 25 mg tablet dosage form. Accordingly, the partition constraint of the first nutritional product 112-1 is 10 mg or 20 mg and increasing from 20 mg upwards in multiples of 5 mg (e.g., 25 mg, 30 mg, 35 mg, etc.), such that the constraint arises from commercially realizable combinations of the 10 mg and the 25 mg dosage forms of the first nutritional product 112-1.

Furthermore, in some embodiments, the partition constraint includes a minimum serving size of the respective nutritional product 112. In some embodiments, the minimum serving size of the respective nutritional product 112 includes a functional limitation of an instrument utilized in quantifying the nutritional product 112 for a respective provision 128. For instance, if the instrument includes a scale to determine a mass with a precision of 0.1 g, the scale imparts a partition constraint of 0.1 g for one or more nutritional products 112 that require use of the scale for forming the respective provision. In some embodiments, the minimum serving size of the respective nutritional product 112 includes a functional limitation of the respective nutritional product 112, such as a mass of a minimum effective dosage of the nutritional product 112.

In some embodiments, the product data 116 for each respective nutritional product 112 includes an availability of the respective nutritional product 112. In some embodiments, the availability of the respective nutritional product 112 includes an available inventory of the respective nutritional product 112 (e.g., an inventory of the respective nutritional product 112 available at the provision preparation environment 30 of FIG. 1). In some embodiments, the availability of the respective nutritional product 112 includes a shelf life associated with the respective nutritional product 112 (e.g., a best-by date), or similar metric quantifying temporal limitations for consumption of the respective nutritional product 112. In some embodiments, the availability of the respective nutritional product 112 includes a geographic availability, such as an indication of a geographic restriction for the respective nutritional product 112, an indication of a physical location associated with the respective nutritional product 112, a seasonality associated with the respective nutritional product 112, or a combination thereof.

In some embodiments, the product data store 110 includes a variety of classification data 118 associated with a respective nutritional product 112. The classification data 118 identifies one or more classifications 118 associated with the respective nutritional product 112 (e.g., a first classification 118-1 is associated with a first nutritional product 112-1 and a third nutritional product 112-3; a second classification 118-2 is associated with the first nutritional product 112-1 and a fourth nutritional product 112-4; etc.). In some embodiments, each respective classification 118 is associated with a predetermined dietary recommendation 126, such that a first classification 118-1 is associated with a first dietary recommendation 126-1; a second classification 118-2 is associated with a second dietary recommendation 126-2; etc. The predetermination dietary recommendation 126 associated with a respective classification 118 provides groupings of one or more nutritional products 112 that commonly associated with each other. Accordingly, the recommendation server system 200 modifies one of the predetermined dietary recommendations in response to one or more responses 132 provided by a respective user, reducing an amount of computational processing required by the recommendation server system 200. In some embodiments, one or more classifications 118 is associated with a respective subset of nutritional products 112, such as a subset of nutritional products 112 including a first dietary supplement 114-1.

Referring back to FIG. 1 through FIG. 2B, in some embodiments, the recommendation server system 200 includes the user response data store 130. The user response data store 130 is configured to store various data related to various population of users and/or one or more respective users. For instance, in some embodiments, the user response data store 130 stores one or more assessment responses 132 that a respective user provides for an assessment survey (e.g., assessment 162 and responses 132). Using these stored responses 132, the recommendation server system 200 can modify future assessment surveys 162 (e.g., reassessment surveys) responsive to these stored response 132. For instance, if a respective response 132 from a first user 10-1 describes an age of the first user 10-1, future assessment surveys 162 need not include an assessment prompt 164 that elicits an age of the first user 10-1.

In some embodiments, the user response data store 130 stores one or more dietary recommendation 126 including the dietary supplement 114 options, and optionally the selected dietary supplements 114 from the dietary supplement 114 options, that were previously provided to a respective user. In some embodiments, the recommendation generation module 120 utilizes this stored recommendation choice data to refine generating additional dietary recommendations 126, e.g., by determining patterns in provision 128 choices made by the respective user, and/or ranker module 140, which uses the recommendation choice data to inform future selection of individualized dietary recommendations 126 from global dietary recommendations, such as classifications 118 (e.g., generated by dietary recommendation generation module 120), e.g., by determining patterns in assessment responses 132 of each respective user. In some embodiments, the user response data store 130 includes assessment responses 132 provided by respective users in a population of users, such as collectively storing responses 132 provided by users in a population of vegan users. Accordingly, the recommendation server system 200 determines one or more underlying trends of the respective population of users through the collective responses 132, such that future assessment surveys 162 provided to the respective population of users are modified in light of these determined treads (e.g., a determined trend of increases in a turmeric dietary supplement 114-1 for various provisions 128 associated with a first population of users yields dietary recommendations 126 including the turmeric dietary supplement 114-1).

In some embodiments, the user response data store 130 stores various dietary information related to each respective users in the population of users, e.g., a diet type of a respective user, a dietary restriction of the respective user, a dietary preference of the respective user, a health related goal of the respective user, etc., which is accessible by, e.g., recommendation generation module 120, which uses the dietary information to inform generation of recommendations, e.g., by accounting for the dietary needs of a respective user associated with a respective provision preparation environment 30, and/or ranker module 140, which uses the dietary information to inform selection of personalized dietary recommendations 126 from a selection of predetermined dietary recommendations 126, such as classifications 118, (e.g., generated by recommendation generation module 120), e.g., by matching the respective user's dietary requirements to respective available dietary recommendations 126.

In some embodiments, the user recommendation data store 130 stores various user preferences such as a dietary religious preference (e.g., a preference for kosher nutritional products 112, a preference for halah nutritional products, etc.). In some embodiments, the user preferences include a preference for nutritional products 112 that satisfy a vegetarian diet, a vegan diet, a pescatarian diet, a gluten-free diet, a diary-free diet, a nut-free diet, a soy-free diet, a caffeine-free diet, a ketogenic diet, a paleolithic diet, or a combination thereof. In some embodiments, the user preferences for a respective user include a listing of one or more preferred (e.g., favorite) and/or disliked products (e.g., the respective user has a distaste for vanilla flavored nutritional products 112). In some embodiments, the user preferences for a respective user include information related to one or more medical conditions associated with the respective user (e.g., a note that a user is diabetic, a note that a user is allergic to nuts). In some embodiments, the one or more medical conditions associated with the respective user is determined through analysis of a biological sample of the respective user, or derived from an electronic medical record associated with the respective user. Furthermore, in some embodiments, the user preferences for a respective user includes one or more nutritional product 112 source preferences (e.g., a preference for organic nutritional products 112, a preference for nutritional products 112 sourced from a specific location, a preference for nutritional products 112 from a specific entity (e.g., a preference for a specific brand), etc.). In some embodiments, the user preferences of the user recommendation data store 130 are applied as filters in determining a dietary recommendation 126 for a respective user (e.g., user viability 324 and/or nutrition criterion 342 of FIG. 3).

In some embodiments, the user recommendation data store 130 stores a variety of data associated with one or more characteristics of respective users. In some embodiments, a biological sample is obtained from a respective user and utilized to determine the one or more characteristics of the respective user. Similarly, in some embodiments, the one or more characteristics of a respective user is obtained from an electronic medical record associated with the respective user.

In some embodiments, the one or more characteristics of a respective user includes one or more anthropometric data points. The one or more anthropometric data points include a current weight of a respective user, a desired weight of the respective user, a previous weight of the respective user, a height of the respective user (e.g., a standing height, a knee height, etc.), or a combination thereof. In some embodiments, the one or more anthropometric data points includes a circumference of a portion of the respective user, such as a hip circumference or a thigh circumference.

Furthermore, in some embodiments, the one or more characteristics of a respective user includes one or more physiologic data points of the respective user. In some embodiments, the one or more physiological data points include an allergy of the respective user to a specific nutritional product 112 and/or a specific dietary supplement 114. In some embodiments, the one or more physiological data points includes a variety of genetic data and/or metabolomics data of the respective user. In some embodiments, the physiological data of the respective user includes an organ function and/or functional capacity of an organ for the respective user. Accordingly, a dietary recommendation 126 for the respective user can exclude one or more nutritional products 112 that have an adverse effect identified through the physiological data.

In some embodiments, the various data of the user recommendation data store 130, including the provided assessment responses 132, is utilized to determine one or more health related metrics for a respective user. Health related metrics include a body mass index and/or a caloric target (e.g., a caloric intake) of the respective user. In some embodiments, the health related metrics further include a body fat metric (e.g., body fat percentage, fat-free body weight, subcutaneous fat percentage, visceral fat percentage, water weight percentage, skeletal muscle mass, bone pass, metabolic age, and the like. In some embodiments, the caloric intake of the respective user includes a basal metabolic rate (BMR). In some embodiments, the caloric intake of the respective user includes applying (e.g., multiplying) an activity factor variable (e.g., an activity level of the respective user determined through one or more responses 132) with the basal metabolic rate (BMR). In some embodiments, the one or more health related metrics includes a current metric that utilizes data related to a current state of the respective user (e.g., a current weight of 85 kilograms (kg) on the day of obtaining the data), a projected metric that utilizes data related to a desired state of the respective user (e.g., a desired weight goal of 75 kg), a previous metric that utilizes historical data (e.g., previously obtained data) of the respective user (e.g., a previous weight of 95 kg on a day of obtaining a previous response to a previously provided assessment survey 162), or a combination thereof.

In some embodiments, the recommendation server system 200 includes the ranker module 140 that at least provides a hierarchy of one or more nutritional products 112 from the product data store 110. In some embodiments, the ranker module 140 determines a hierarchy of the one or more nutritional products 112 based on a number of votes cast against each respective nutritional product 112 in the one or more nutritional products 112. For instance, in some embodiments, the ranker module 142 ranks each nutritional product 112 such that a corresponding nutritional product 112 that received a highest number of votes is ranked first, and additional nutritional products 112 are ranked in descending order according to the number of votes received against the respective nutritional products 112. Since each vote cast against a respective nutritional product 112 by a decision rule 112 is an indication that the respective nutritional product 112 is beneficial for a user, the ranking provided by the ranker module 140 allows the most beneficial nutritional products 112 to have a higher priority for inclusion in a dietary recommendation 126.

Moreover, in some embodiments, the ranker module 140 forms one or more groupings of nutritional products 112, which each nutritional product in a respective group being ranked. For instance, in some embodiments, the ranker module 140 forms a first ranking of one or more nutritional products 112 determined to be essential for a dietary recommendation 126, and a second ranking of one or more nutritional products 112 determined to be optional for the dietary recommendation 126. These separate rankings allows the user, or similarly the recommendation server system 200, to consider each separate hierarchy and select nutritional products 112 depending on specific preferences. In some embodiments, the one or more groupings of nutritional products 112 is one or more classifications 118 of nutritional products 112.

In some embodiments, the ranker module 140 includes one or more decision rules 122, similar to those described with respect to the recommendation generation module 120, that are used in providing a ranking of nutritional products 112 that received votes. In some embodiments, a respective decision rule 122 of the ranker module 140 provides a rule for situations in which two or more nutritional products 112 receive the same number of votes. As an example, if a first nutritional product 112-1 and a second nutritional product 112-2 receive the same number of votes, a corresponding decision rule 122 requires that respective nutritional products 112 associated with specialty tags 124 have a higher ranking than respective nutritional products 112 associated with vitamin tags 124, which have a higher ranking than respective nutritional products 112 associated with mineral tags 124.

In some embodiments, a respective decision rule 122 of the ranker module 140 provides a rule for ranking one or more predetermined nutritional products. As an example, if a fourth nutritional product 112-4 associated with specialty tags 124 receives a number of votes that ranks the fourth nutritional product 112-4 in the top five nutritional product 112, a corresponding decision rule 122 requires that the fourth nutritional product 112-4 cannot appear within the top five nutritional products 112 and lowers the ranking of the fourth nutritional product 112-4 accordingly. In some embodiments, a respective decision rule 122 of the ranker module 140 provides a rule for modifying the ranking in accordance with a corresponding assessment response 132. For instance, in some embodiments, if a respective assessment response 132 is provided a user, a corresponding decision rule 122 is fired to modify the ranking of the subset of nutritional products 112.

In some embodiments, a dietary recommendation 126 includes the determined hierarchy of the one or more nutritional products 112. By including the determined hierarchy of the one or more nutritional products 112, a respective user can select various nutritional products 112 from the hierarchy to form a corresponding provision 128 of the dietary recommendation 126. As an example, consider a dietary recommendation 126 for a respective user that determines a first nutritional product 112-1 is best for the user, and also provides a hierarchy describing a second nutritional product 112-2 and a third nutritional product 112-3 as further benefiting the respective user but less so than the first nutritional product 112-1. Accordingly, the respective user is allowed to include the second nutritional product 112-2 and/or the third nutritional product 112-3 in a provision 128 of the dietary recommendation 126, either in lei of or supplemental to the first nutritional product 112-1.

In some embodiments, the recommendation server system 200 includes the nutrient data store module 150 that stores a variety of dietary supplement reference data 152. Each respective dietary supplement reference data 152 includes one or more nutrient metrics 154 that describe a nutritional parameter or characteristic related to a corresponding dietary supplement 114 associated with a respective dietary supplement 152, such as a periodic nutritional limit.

In some embodiments, the recommendation server system 200 includes the assessment store 160 that stores the assessment surveys 162 provided to a population of users. In some embodiments, the assessment store 160 includes a number of unique assessment prompts 164 (e.g., a first assessment prompt 164-1, a second assessment prompt 164-2, a third assessment prompt 164-3, etc.), with each unique assessment prompt 164 configured to elicit a response from a respective user (e.g., response 132 of FIG. 1). In some embodiments, a respective assessment survey 162 is formed from an amalgamation of one or more assessment prompts 164 stored in the assessment store 160. As such, the assessment store 160 forms a database of assessment prompts 164 that can be selected to form an assessment survey 162. For instance, in some embodiments, a first user 10-1 is provided with a first assessment survey 162-1 including a first plurality of assessment prompts 164 (e.g., a first assessment prompt 164-1 and a second assessment prompt 164-2), and a second user 10-2 is provided with a second assessment survey 162-2 including a second plurality of assessment prompts 164 (e.g., the first assessment prompt 164-1 and a fourth assessment prompt 164-4).

In some embodiments, each respective assessment prompt 164 is associated with an assessment group 168, which forms a shared association of related assessment prompts 164 via the same assessment group 168. In some embodiments, a respective assessment group 168 is associated with a corresponding survey 162, such that each assessment prompt 164 associated with the respective assessment group 162 forms all or a part of the corresponding survey 162.

Moreover, in some embodiments, each respective assessment group 168 is associated with a unique subset of assessment prompts 164 that are configured to elicit responses 132 from a user that are related to the same subject matter. Furthermore, in some embodiments, each assessment prompt 164 is only associated with a single assessment group 168. Accordingly, in some embodiments, the assessment groups 168 include a biometric assessment group 168-1, a life-stage assessment group 168-2, a physiological assessment group 168-3, a dietary assessment group 168-4, a lifestyle assessment group 168-5, a behavioral assessment group 168-6, a health goal assessment group 168-7, or a combination thereof.

In some embodiments, an assessment survey 162 provides a user with each respective assessment prompt 164 associated with a first assessment group 168-1 prior to providing the user with each respective assessment prompt 164 associated with a second assessment group 168-2. This allows the survey 162 to providing the user with assessment prompts 162 from different groups 168 depending on the responses 132 to a previously provided assessment group 168. For instance, in some embodiments, a first user 10-1 is provide with each respective assessment prompt 162 associated with a first assessment group 168-1. In accordance with a determination that the user responses 132 satisfy a specified condition (e.g., the user provided a certain response 132 to a respective assessment prompt 164), the survey provides the user with each respective assessment prompt 162 associated with a third assessment group 168-1, bypassing the second assessment group 168-2.

In some embodiments, one or more of the above-identified data elements or modules of the recommendation system 200 are stored in one or more of the previously described memory devices (e.g., memory 202 and/or memory 290) and correspond to a set of instructions for performing a function as described above. The above-identified data, modules, and programs (e.g., sets of instructions) need not be implemented as separate software programs, procedures, or modules, and thus various subsets of these modules may be combined or otherwise re-arranged in various embodiments. In some embodiments, the memory 202 and/or memory 290 optionally stores a subset of modules and data structures identified above. Furthermore, in some embodiments, the memory 202 and/or memory 290 stores additional modules and data structures not described above. For instance, in some embodiments, the product data store 110, the recommendation generation module 120, the ranker module 140, the nutrient data store 150, or a combination thereof are subsumed as one module.

As illustrated in FIG. 1, various communications are exchanged in the system 100 between the system 100 and the recommendation system 200 as well as the various modules and devices of these collective systems. Additional details and information regarding the communications between the distributed system 100, the recommendation system 200, the user devices 10, and the modules and the devices of these collective systems will be described in more detail infra.

Now that a general topology of the distributed system 10 has been described, methods for generating dietary recommendations 126 for a user will be described in conjunction with FIG. 5A and FIG. 5B.

Block 502. In more detail, referring to FIG. 5A, one aspect of the present disclosure provides systems (e.g., distributed system 100 of FIG. 1) and methods for generating (e.g., generate assessment 302 of FIG. 3) and providing dietary recommendations (e.g., dietary recommendations 126 of FIG. 1; provide recommendation 350 of FIG. 3). The systems include one or more processors (e.g., CPU 274 of the recommendation system 200 of FIG. 2A) and memory (e.g., memory 202 of the recommendation system 200 of FIG. 2A), that is addressable by the one or more processors. The memory stores one or more programs (e.g., modules of the recommendation system 200 of FIG. 2A) for execution by the one or more processors. The one or more programs includes a variety of instructions for performing the method 500.

Block 504. The method 500 includes obtaining (e.g., via communications network 20 of FIG. 1) one or more assessment responses (e.g., assessment response 132 of FIG. 1) from a respective user (e.g., user device 10-1 of FIG. 1; user data 308 of FIG. 3). The one or more assessment responses 132 is provided in response to a survey (e.g., an assessment survey 162 of FIG. 1) communicated to the respective user device 10. Typically, the assessment responses 132 are obtained from the users devices 10 in electronic form (e.g., the assessment survey 162 is presented to the user through a graphical user interface on a display of the respective user device 10, similar to display 282 of the recommendation system 200 of FIG. 2A). However, the present disclosure is not limited thereto (e.g., in some embodiments, the assessment responses 132 are obtained from the users in a written form or are orally communicated).

In some embodiments, the assessment survey 162 includes a variety of assessment prompts 164 (e.g., assessment questions) that each elicit a respective response 132 from a user. In some embodiments, each respective user provides a response 132 to each assessment prompt 164 of an assessment survey 162. For instance, in some embodiments, the user is required to provide a response 132 for each assessment prompt 164 of the assessment survey 162 in order to receive a dietary recommendation 126. However, the present disclosure is not limited thereto. In some embodiments, each respective user is provided an opportunity to bypass one or more assessment prompts 164 of the assessment survey 162. Optionally, the assessment survey 162 requires the respective user to provide a response 132 for a previously bypassed assessment prompt 164. In some embodiments, the assessment survey 162 provides each respective assessment prompt 164 to the respective user in a sequential order, such that a second assessment prompt 164-2 is provided to the user in accordance with a determination that the user provided a response 132 to a first assessment prompt 164-1. For instance, a first user 10-1 provides a first response 132-1 to the first assessment prompt 164-1 of a first assessment survey 162-1. Responsive thereto, and, based on the value of the first response 132-1, the first assessment survey 162-1 provides a second assessment prompt 164-2 to the first user 10-1. On the other hand, a second user 10-2 provides a second response 132-2 to the first prompt 164-1 of the first assessment survey 162-1. Responsive thereto, and based on the value of the second response 132-2, the first assessment survey 162-1 provides a third assessment prompt 164-3, instead of the second assessment prompt 164-2, to the second user 10-2, bypassing the second assessment prompt 164-2 for the second user 10-2 base on the second user's response to the first prompt 164-1. For instance, the first user's response to the first assessment prompt 164-1 could be a “Yes,” thus causing the first assessment survey to ask question 164-2, while the second user's response to the first assessment prompt 164-1 could be a “No,” thus causing the second assessment survey to skip to question 164-3.

In some embodiments, the assessment survey 162 includes one or more multiple choice prompts (e.g., assessment prompts 164 of FIG. 2B), such that each respective assessment prompt 164 includes a variety of predetermined responses 132 available to the user. In some embodiments, the assessment survey 162 includes one or more free answer prompts 164, such that a user is free to provide any information in response 132 to the prompt 164. For instance, in some embodiments, a prompt 164 elicits a response 132 if the user is subject to one or more dietary restrictions and includes one or more predetermined dietary restrictions for the user to select from (e.g., a first response 132-1 associated with a lactose restriction, a second response 132-2 associated with a kosher restriction, a third response 132-3 associated with no restrictions, etc.). In some embodiments, a respective response 132 to a free answer assessment prompt 164 is evaluated to categorize the respective response 132 to one or more predetermined responses 132. For instance, if the free answer assessment prompt 164 elicits a numerical response 132 from a user (e.g., a first assessment prompt 164-1 eliciting a response 132 associated with a weight of the user), the numerical response 132 provided by the user is evaluated against the one or more predetermined responses (e.g., the response 132 associated with the weight of the user is evaluated against a range of weights of similar types of users and categorized as a normal weight, underweight, overweight, obese, and the like).

In some embodiments, the assessment survey 162 provided to a user includes a variety of assessment prompts 1644. Each assessment prompt 1644 is configured to elicit a specific response 132 describing one or more physiological characteristics of a user by mapping each plausible response 132 for a respective prompt 164 to one or more tags 124.

In some embodiments, the population of users is provided with a first survey 162, such that each user device 10 receives the same assessment survey 162. In some embodiments, the population of users is provided with a first survey prompt 164-1 of an assessment survey 162 which, depending on a response 132 provided by each respective user, provides a corresponding related prompts 164. For instance, in some embodiments, a prompt asks the user whether they are subject to one or more dietary restrictions and includes two predetermined responses for the user to select from (e.g., a first response 132-1 for a positive indication of one or more dietary restrictions and a second response 132-2 for a negative indication of one or more dietary restrictions). Accordingly, if the user provides the first response 132-1 the user is provided a second prompt 164-2 that asks for the dietary restrictions of the user, and if the user provides the second response 132-2 the second prompt 164-2 is bypassed and the user is provided with the next question in the survey, e.g., a third prompt 164-3. Note that in this example the user is provided with the next question in the survey (the third prompt 164-3) regardless of their answer to the dietary restriction answer, it is just that in cases where the user has a dietary restriction, they are first given prompt 164-2.

In some embodiments, the assessment survey 162 includes one or more prompts 164 including one or more biometric assessment prompts 164. The biometric assessment prompts 164 elicit assessment responses 132 that are directed towards describing and determining various physical characteristics of the user, such as anthropometric data. In some embodiments, the one or more biometric assessment prompts 164 elicit corresponding responses 132 including an age of the user, a sex of the user (e.g., male or female), a height of the user, a weight of the user, or a combination thereof.

In some embodiments, the assessment survey 162 includes one or more prompts including one or more life-stage assessment prompts that describe features related to a chronological and/or a biological age of the user. In some embodiments, the one or more life-stage prompts is a subset of the one or more biometric assessment prompts. As described supra, in some embodiments, a respective prompt provided to a user depends on a response to a previous prompt (e.g., a first user 10-1 provides a first response to a first prompt that leads to a third prompt, and a second user 10-2 provides a second response to the first prompt that leads to a second prompt). In some embodiments, in accordance with a determination that one or more respective assessment responses 132 obtained from the user indicates an age in a predetermined range (e.g., from 20 years of age to 45 years of age) and a sex of female, the life-stage assessment prompts 164 elicit a response including whether the subject is pregnant, breastfeeding, or has an experience of physical or emotional symptoms. In some embodiments, in accordance with a determination that one or more assessment responses obtained from the subject indicates an age greater than a threshold number (e.g., 45 years of age) and a sex of female, the life-stage assessment prompts 164 further elicit a response including whether the subject has an experience of physical or emotional symptoms or menopause related symptoms.

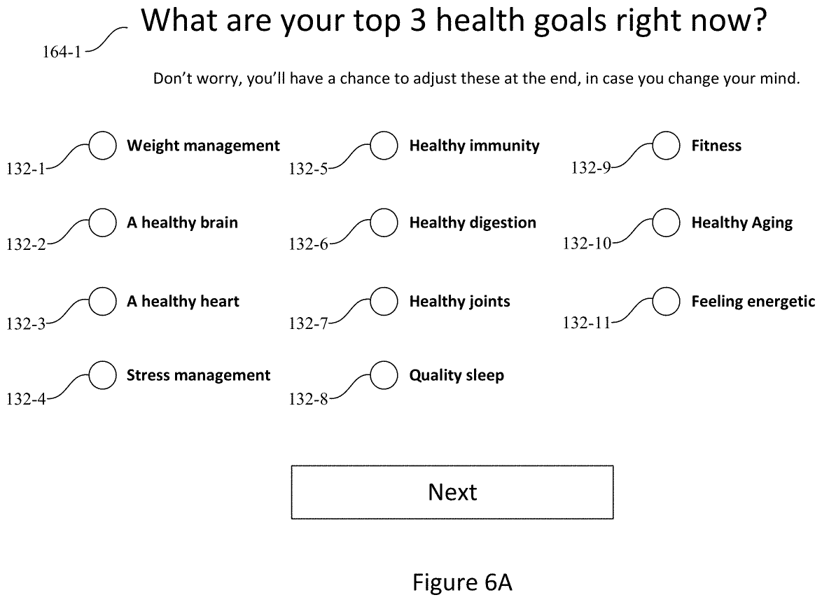

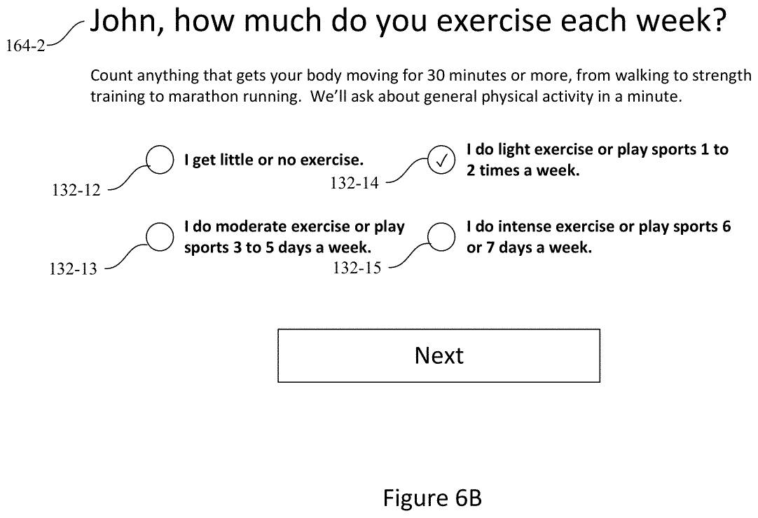

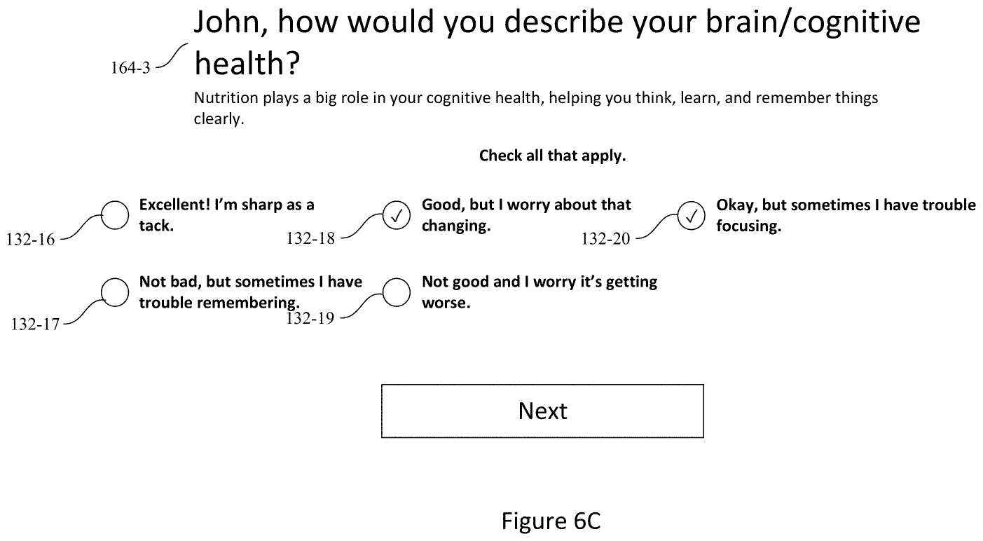

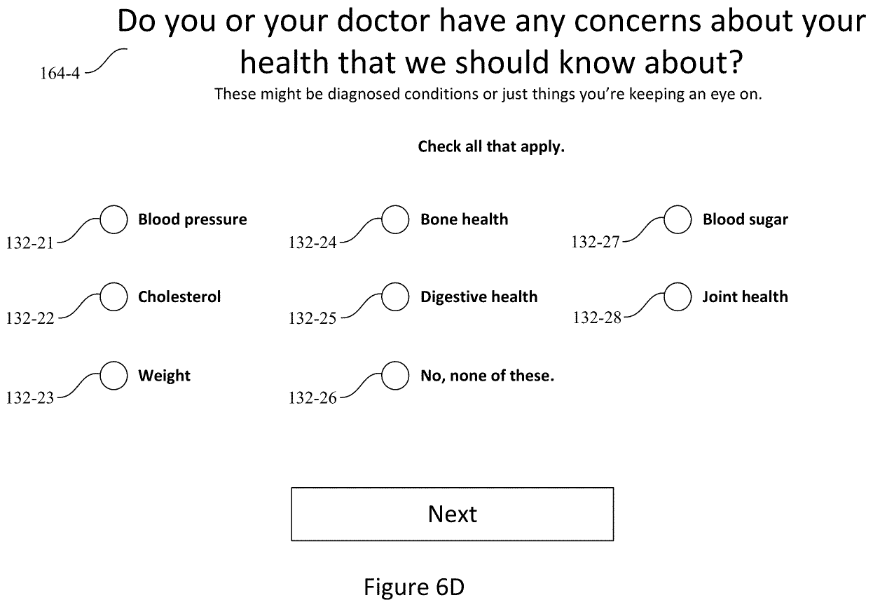

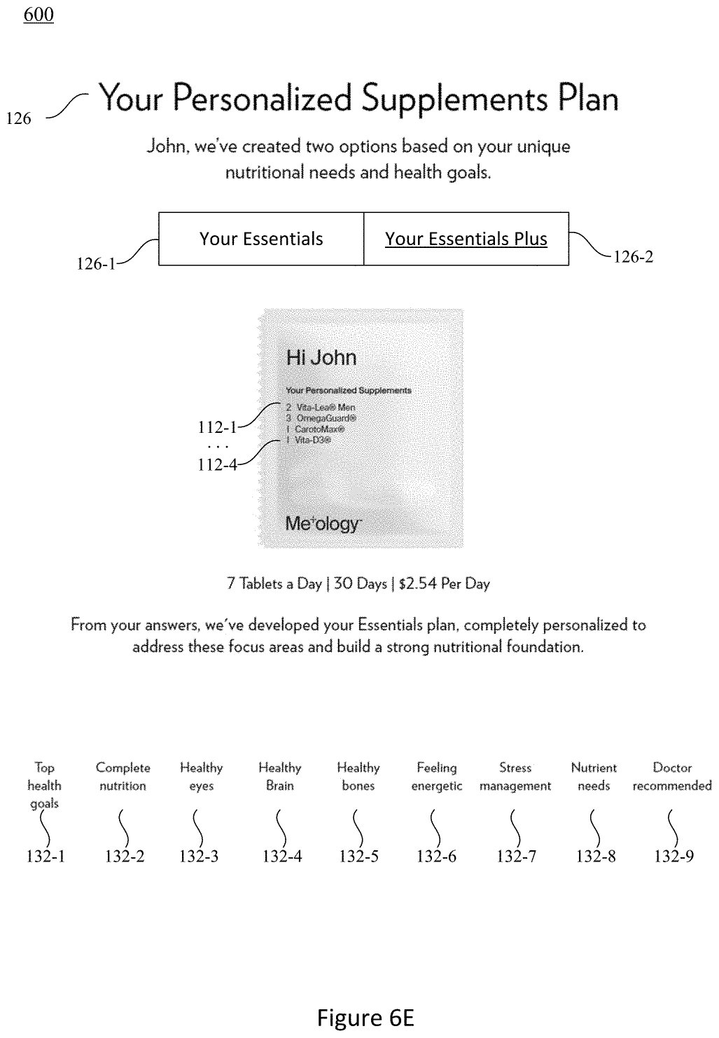

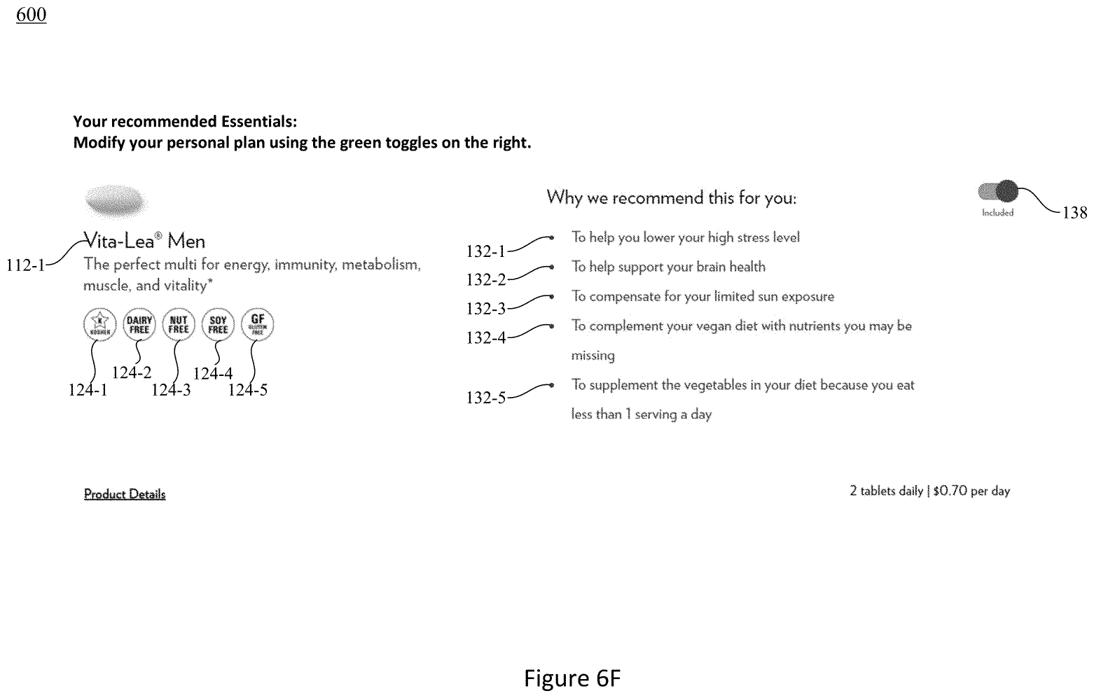

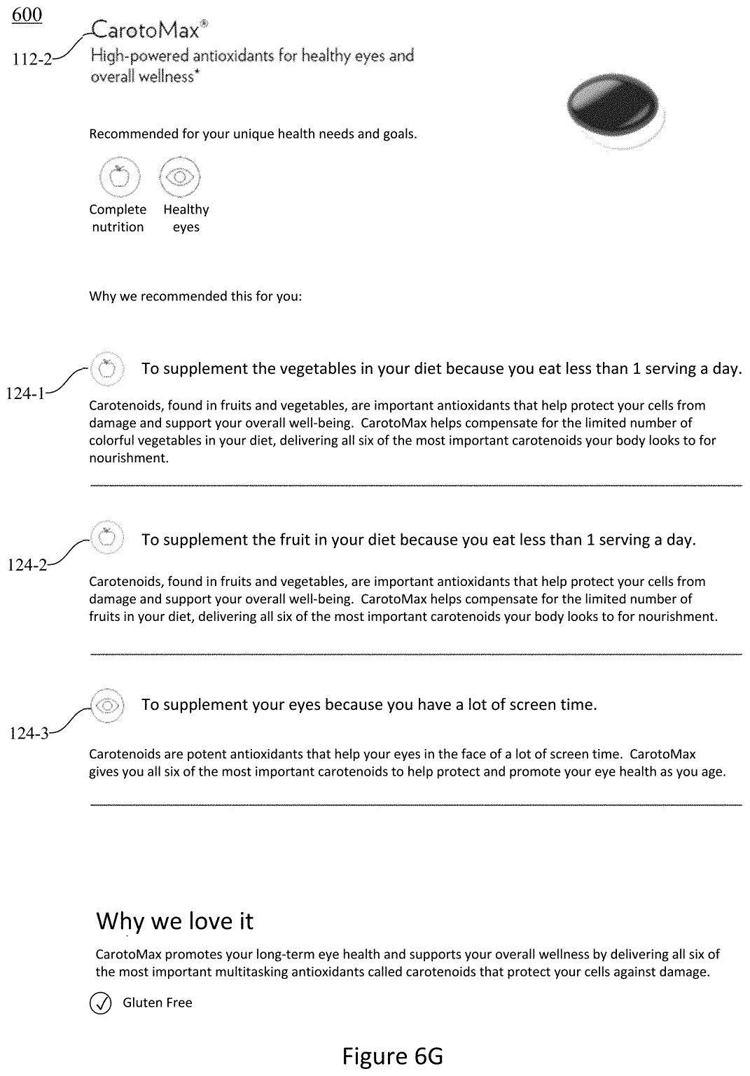

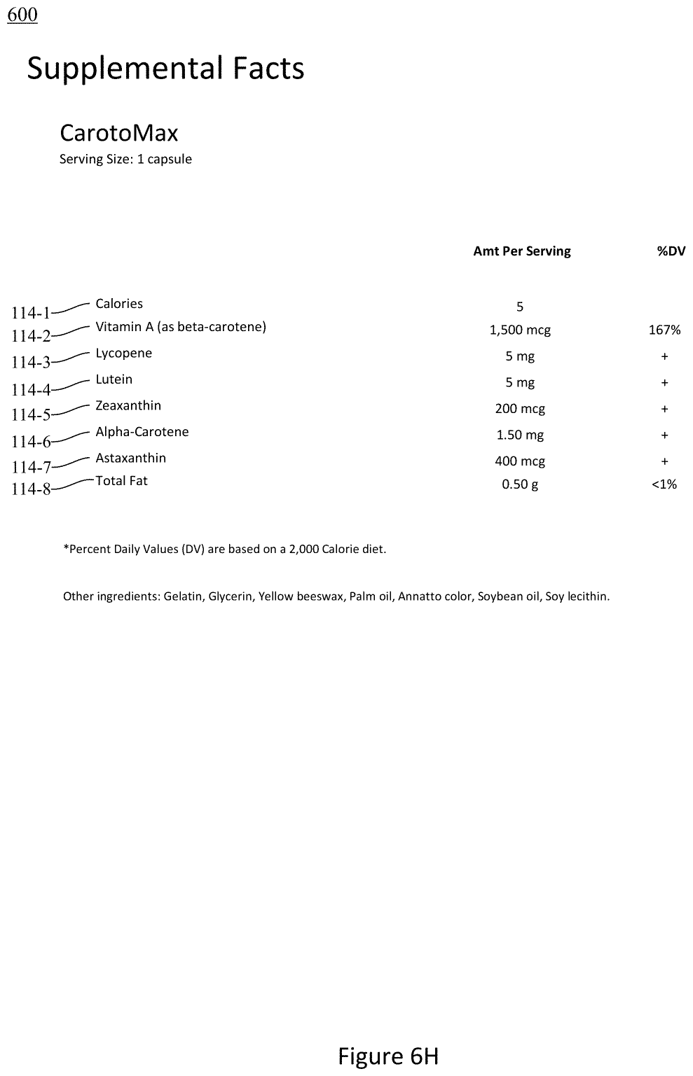

In some embodiments, the assessment survey 162 includes one or more prompts 164 including one or more physiological assessment prompts. In some embodiments, the physiological assessment prompts elicit corresponding assessment responses 132 including: whether a health practitioner associated with the user has indicated a concern associated with the user for one or more health conditions, a familial health condition history, whether the health practitioner associated with the user has indicated a recommendation for one or more dietary supplements (e.g., one or more nutritional products 112), and whether the user is currently taking a pharmaceutical composition (e.g., a pharmaceutical composition having an adverse reaction to one or more nutritional products 112 and/or one or more dietary supplements 114). By way of example, referring briefly to FIG. 6D, an assessment prompt 164 for whether a health practitioner associated with the user has indicated a concern elicits a selection of assessment responses 132 including a concern for: a blood pressure of the subject 132-1, a cholesterol of the subject 132-2, a weight of the subject 132-3, a bone health of the subject 132-4, a digestive health of the subject 132-5, a blood sugar level of the subject 132-7, a joint health 132-8, or a combination thereof.

In some embodiments, the one or more health conditions of concern includes a cholesterol level of the user, the weight of the user, a blood sugar level of the user, a blood pressure level of the user, a bone health metric of the user (e.g., a bone mineral density), a digestion metric of the user, a joint health of the user, or a combination thereof. In some embodiments, the one or more health conditions is stored in the user response data store 130. In some embodiments, in accordance with a determination that one or more assessment responses 132 obtained from the subject indicates the blood sugar level health condition concern or the weight health condition concern, the physiological assessment prompts 164 further elicit a response including a diabetes status of the subject. In some embodiments, the familial health condition history including an indication of a familial history of high cholesterol, a familial history of cardiovascular disease, a familial history of high blood pressure, a familial history of osteoporosis, or a combination thereof. In some embodiments, the pharmaceutical composition indicated by the user includes a cholesterol lowering pharmaceutical composition, a blood thinning pharmaceutical composition, a blood pressure pharmaceutical composition, an acid-suppressing pharmaceutical composition, or a combination thereof.

In some embodiments, the assessment survey 162 includes one or more prompts 164 including one or more dietary assessment prompts 164. In some embodiments, the dietary assessment prompts 164 elicit a corresponding assessment responses 162 including whether the user has one or more dietary restraints and/or a number of servings of one or more respective dietary supplements 114 consumed by the user. In some embodiments, the number of servings of one or more respective dietary supplements includes a number of vegetable servings consumed by the subject, a number of fruit servings consumed by the subject, a number of diary servings consumed by the subject, a number of omega-3 fatty acid servings consumed by the subject, or a combination thereof.

In some embodiments, the one or more dietary restraints include an indication if the corresponding restraint is a personal preference of the subject or a medical requirement. In some embodiments, the one or more dietary restraints include a vegetarian restrain, a vegan restraint, a pescatarian restraint, a gluten intolerance restraint, a dairy intolerance restraint, a nut intolerance restraint, a soy intolerance restraint, a kosher restraint, a ketogenic restraint, a paleolithic restraint, a caffeine restraint, or a combination thereof.

In some embodiments, the assessment survey 162 includes one or more prompts 164 including one or more lifestyle assessment prompts 164. In some embodiments, the life-style assessment prompts 164 elicit corresponding assessment responses 132 including a level of physical activity endured by the user, whether the user physical exerts themselves at a work, an energy level of the subject, a stress level of the user, a sleeping habit of the user, a cognitive health assessment of the user, a sun exposure level of the user, a computer use level of the user, or a combination thereof. In some embodiments, the life-style assessment prompts 164 are relative to a period of time (e.g., computer use level of the user per day). For instance, referring briefly to FIG. 6B, an assessment prompt 164 associated with a lifestyle of an amount of exercise each week elicits a fourteenth assessment response 132-14 associated with a light exercise activity of the subject. In some embodiments, in accordance with a determination that one or more assessment responses 132 obtained from the user satisfies a first threshold level of physical activity, the life-style assessment prompts 164 further elicit an assessment response 132 including an indication of a type of physical activity endured by the subject. As another non-limiting example, referring briefly to FIG. 6C, in some embodiments, an assessment prompt 164 associated with a lifestyle of a cognitive health assessment of the user is presented. This assessment prompts elicits an assessment response 132 that includes a first assessment response 132-1 associated with a moderate cognitive health and a second assessment response 132-2 associated with a difficulty focusing.

In some embodiments, in accordance with a determination that one or more assessment responses 132 obtained from the user satisfies a second threshold level of physical activity, the life-style assessment prompts 164 further elicit an assessment response 132 including an indication of a desired health goal from consuming a respective nutritional product 112. In some embodiments, the desired health goal includes optimizing a performance of the physical activity, optimizing an aspect of muscle growth, reducing an aspect of muscle soreness, replenishing one or more dietary supplements 114, or a combination thereof.

In some embodiments, the assessment survey 162 includes one or more prompts 164 including one or more behavioral assessment prompts 164. In some embodiments, the behavioral assessment prompts 164 elicit corresponding assessment responses 132 including whether the user is currently taking a nutritional supplement (e.g., a nutritional product 112 of the product data store 110). In some embodiments, in accordance with a determination that one or more assessment responses 132 obtained from the subject indicates that the subject is currently taking a nutritional supplement, the behavioral assessment prompts 164 further elicit an assessment response including a number of nutritional supplements (e.g., nutritional products 112 and/or dietary supplements 114) that the user is currently taking. In some embodiments, in accordance with a determination that one or more assessment responses 132 obtained from the subject indicates the subject is currently taking a nutritional supplement, the behavioral assessment prompts 164 further elicit an assessment response 132 including a role of the nutritional supplement the subject is currently taking.

In some embodiments, the assessment survey 162 includes one or more prompts 164 including one or more health goal assessment prompts 164. In some embodiments, the health goal assessment prompts 164 elicit a corresponding assessment response 132 including an indication of one or more health related interests of the user. In some embodiments, the indication of one or more health related interests includes a ranking of the one or more health related interests. By way of example, referring briefly to FIG. 6A, a subject is presented with an assessment prompt 164 that elicits a response from the subject for three health goals of the subject. Here, the subject is presented with eleven various user responses 132 that each correspond to a unique health goal (e.g., first assessment response 132-1 corresponds to first health goal of weight management health interest, second assessment response 132-2 corresponds to second health goal of healthy brain health interest, . . . , eleventh assessment response 132-11 corresponds to eleventh health goal of feeling energic health interest, etc.). In response, the subject provides a selection of an indication of three health related interests from the eleven various user responses 132. In some embodiments, the ranking of the one or more health related interests modifies a weight of a vote cast by one or more decision rules 122. As an example, consider a user providing primary health goal of stress management and a secondary health goal of improving sleep quality. Accordingly, each vote cast for a respective nutritional product 112 associated with the primary health goal has a weight increased by a first factor (e.g., three), and each vote cast for a respective nutritional product 112 associated with the second health goal has a weight increased by a second factor that is less than the first factor (e.g., two). Now consider a first nutritional product 112-1 associated with the primary health goal, a second nutritional product 112-2 associated with the second primary health goal, and a third nutritional product 112-3 associated with the primary and secondary health goals. If a respective decision rule fires and casts a vote against each of the first nutritional product 112-1, the second nutritional product 112-2, and the third nutritional product 112-3; the third nutritional product 112-3 will be ranked highest with weighted votes totally five (e.g., an initial vote increased by association with the primary health goal and further increased by association with the second health goal) and the second nutritional product 112-2 will be ranked last (e.g., an initial vote increased by association with the secondary health goal). As another example, if a medical practitioner associated with a user is concerned about the blood pressure of the user, the responses 132 with tag to an omega 3 tag 124 associated with a 1,000 mg dosage. However, if the user responses 132 indicate that the user consumes zero servings of fatty fish a week, then the omega 3 tag 124 described above actually tags to a different tag associated with a different dosage form of omega three, such as three dosages of the different dosage form. Similarly, if the user responses indicate that the user consumes one serving of fatty fish a week, then their above described omega 3 tag 124 tags to a the other omega 3 tag, optionally with a lower dosage that the if the response indicated that the user consumed zero fatty fish. Accordingly, depending on user responses 132 to one or more assessment prompts 164, either a type of nutritional product 112 or dosage of a respective nutritional product is modified accordingly.

In some embodiments, when a respective assessment response 132 obtained from the user is a first assessment response 132-1, a related assessment prompt 164 in the assessment survey 162 is bypassed. For instance, if a user provides a first assessment response 132-1 indicating the user is male in response to a first assessment prompt 164-1, a related second assessment prompt 164-2 concerning the pregnancy status of the user is bypassed. In some embodiments, the user is provided a third assessment prompt 164-3, for instance an assessment prompt 164 related to males, responsive to providing the first assessment response 132-1.

In some embodiments, a variety of anthropometric data associated with the user is obtained. In some embodiments, the anthropometric data is derived from a biological sample obtained from the user (e.g., a saliva sample obtained from the user) and/or one or more responses 132 to the survey 162 provided by the user. In some embodiments, the anthropometric data further includes one or more user preferences associated with the user of the user device 10. For instance, in some embodiments, the anthropometric data includes an indication that the user is allergic to one or more dietary supplements 114, which forms a user preference to exclude the one or more dietary supplements 114. In some embodiments, the anthropometric data further includes a variety of genetic data and/or a variety of metabolomics data.

In some embodiments, the survey 162 is an assessment survey for providing an assessment of a user and providing a corresponding dietary recommendation 126. In some embodiments, the survey 162 is a reassessment survey for providing a reassessment of the user and/or a further dietary recommendation 126 in light of an efficacy of a previous dietary recommendation 126 provided for the respective user. Accordingly, in some embodiments the reassessment survey utilizes previous assessment responses (e.g., the user response data store 130) to supplement one or more responses that would otherwise be provided by a respective user. For instance, if a previous assessment response provided by a respective user indicates the user is allergic to a first dietary supplement 114-1, subsequent surveys (e.g., reassessment surveys 162) will omit one or more prompts associated with the first dietary supplement 114-1.

In some embodiments, a physical location associated with the user is obtained as a portion of the assessment survey 162. In some embodiments, the physical location includes an electronic address associated with a respective user device 10 of the user (e.g., an electronic address similar to the electronic address 106 associated with the recommendation server system 200). In some embodiments, the physical location is obtained through an assessment response 132 to a corresponding assessment prompt 164 of the assessment survey 162.

Referring briefly to FIG. 3, and with reference to FIG. 1 through FIG. 2B, the recommendation server system 200 includes the recommendation generation module 120 that generates a corresponding dietary recommendation 126 for each respective user based on a plurality of assessment responses 132 provided by a respective user in response to an assessment 162. The plurality of assessment responses 132 utilized by the recommendation generation module 120 includes each assessment response 132 provided by a respective user or a subset of such responses 132.

Block 506. Some or all of the plurality of assessment responses 132 provided by the user are used to select a corresponding set of tags 124.

In some embodiments, a respective response 132 provided by the user is not utilized in selecting a corresponding set of tags 124, such that the respective response 132 is unassociated with a tag 124. For instance, in some embodiments, a first assessment response 132-1 associated with a first assessment prompt 164-1 is obtained and evaluated by the system 100. In accordance with the evaluation of the first assessment response 132-1, the system 100 determines that a second assessment prompt 164-2 is not applicable to the user, and accordingly provides a third assessment prompt 164-3 of the assessment survey 162 to the user. As such, this first assessment prompt 164-1 is utilized to guide (e.g., progress through) the assessment survey 164 (e.g., to navigate through the survey and which parts of the survey to conduct) instead of selecting one or more tags 124 for inclusion in the set of tags 124. In some embodiments, either all or a subset of the assessment responses 132 provided by the user is used in selecting the corresponding set of tags 124.

In some embodiments, each respective assessment prompt 164 of the assessment survey 162 is associated with one or more tags 124, such that a subset of the one or more tags 124 associated with the respective assessment prompt 164 is selected in accordance with a determination of an assessment response 132 provided by the user. In some embodiments, the respective assessment response 132 is associated with the one or more tags 124 according to an assessment response 132 to tag 124 lookup data structure (e.g., chart 440 of FIG. 4D; the product data store 110, the user response data store 130, the ranker module 140, the dietary supplement data store 150, or a combination thereof).

In some embodiments, the user sequentially progresses through each respective assessment prompt 164, or optionally each respective assessment group 168, of the assessment survey 162. As such, the system 100 forms a first plurality of tags 124 as the user provides each sequential assessment response 132. In some embodiments, if the first plurality of tags 124 satisfies a threshold number of tags 124, the user is considered to satisfy a minimum requirement of the assessment survey 164. As such, the user is provided the opportunity to leave the assessment survey 164 early using just the first plurality of tags 124 that satisfy the threshold number of tags 124, or continue progressing through the assessment survey 162 and further incorporating more tags 124 into the first plurality of tags 124. Accordingly, each set of tags 124 selected by all or a subset of the assessment responses 132 provided by the user collectively form a first plurality of tags 124 for polling. In some embodiments, the first plurality of tags 124 is a collection of each unique tag 124 identified by the assessment response 132 to tag 124 lookup data structure 440.

Referring briefly to FIG. 4D, in some embodiments, the assessment response 132 to tag 124 lookup data structure 440 is formed from the recommendation generation module 120 and other modules of the recommendation system 200 (e.g., the product data store 110, the ranker module 140, the nutrient data store 150, or a combination thereof). For instance, a chart 440 illustrates an example data structured from the recommendation generation module 120 and other modules of the recommendation system 200. The exemplary chart 440 includes a first column 442 that provides a listing of one or more sets of assessment prompts 164 of a respective assessment survey 162. As an example, the first column 442 of chart 440 of FIG. 4D includes a plurality of biology based assessment prompts 164 (e.g., physiological assessment prompts 164) and a plurality of health goal assessment prompts 164 for a respective assessment survey 162. The chart 440 also includes a second column 444 that provides a listing of each respective assessment prompt 164 associated with a corresponding plurality of assessment prompts 164 provide in the respective assessment survey 162. Moreover, a third column 446 provides a listing of each respective assessment response 132 that is associated with a corresponding assessment prompt 164 (e.g., a first assessment prompt 164-1 of the first second column 444 includes associated response 132-1 through response 132-6). As previously described, in some embodiments, a user selects one or more of the responses 132 associated with a corresponding assessment prompt 164, or the method 500 evaluates a response 132 provided by the user and maps the user provided response 132 to one or more predetermined responses 132. In some embodiments, the assessment prompts 164 of the first column 442 and the second column 444 as well as the assessment responses 132 of the third column 446 are provided by the assessment store 160. Still further, the chart 440 includes a fourth column 448 that provides a listing of one or more tags 124 associated with the recommendation server system 200. Each tag 124 listed in the fourth column 448 is either associated with a dietary nutritional product 112 of the product data store 110 and/or a decision rule 122 of the recommendation generation module 120. In the illustrated embodiment, the fourth column 448 is further divided into a first grouping 448-1 of each vitamin tag 124 and a second grouping 448-2 of each mineral tag 124, allowing for individual evaluation of a respective grouping of tags 124. However, the present disclosure is not limited thereto.

The assessment response 132 to tag 124 lookup data structure 440 identifies a first plurality of tags 124 that is collectively associated with the one or more assessment responses 132 utilized in generating a dietary recommendation 126 for the user. The first plurality of tags 124 is identified by selecting each respective set of tags 124 associated a respective assessment response 132 provided by the user. As an example, if a user provided response 132-17 for assessment prompt 164-4 of FIG. 4D, the assessment response 132 to tag 124 lookup data structure 440 identifies an iron tag 124-12 for inclusion in the first plurality of tags 124. By identifying the first plurality of tags 124 that is collectively associated with each of the one or more assessment responses 132, dietary and health related needs of the user derived from the one or more assessment responses 132 are collectively represented by, and evaluated through, the identified first plurality of tags 124. Moreover, since the assessment responses 132 used in generating a dietary recommendation 126 are collectively represented by the first plurality of tags 124, individual assessment responses 132 are collectively evaluated by the recommendation generation module 120 through the first plurality of tags 124, allowing for a more robust dietary recommendation 126 via the interrelation of the individual assessment responses 132 and the first plurality of tags 124 identified by the assessment response 132 to tag 124 lookup data structure.

In some embodiments, one or more respective assessment responses 132 includes predetermined associations with zero or more tags 124. For instance, in some embodiments a respective assessment response 132 is a non-answer response (e.g., response 132-6 “None of the above” of FIG. 4D), or the respective assessment response 132 is a free form response provided by the user. Accordingly, the assessment response 132 to tag 124 lookup data structure 440 identifies a first plurality of tags 124 by selecting each of the zero or more predetermined tags 124 associated with each respective assessment response 132 provided by a user. In some embodiments, the first plurality of tags 124 identified by the assessment response 132 to tag 124 lookup data structure 440 contains each unique tag 124 included in the one or more respective assessment responses 132. For instance, consider a user providing both a first assessment response 132-1 and a second assessment response 132-2. The first assessment response 132-1 (e.g., a response that indicates the subject is male) includes predetermined associations with a first tag 124-1, and the second assessment response 132-2 (e.g., a response that indicates the age of the subject) includes predetermined associations with the first tag 124-1, a third tag 124-3, and a fourth tag 124-4. The assessment response 132 to tag 124 lookup data structure 440 identifies a corresponding first plurality of tags 124 by selecting the first tag 124-1, the third tag 124-3, and the fourth tag 124-4 from the first and second assessment responses 132.

As an example, consider a first assessment prompt 164-1 relating to a question of a number servings of vegetables consumed each day. The user can provide a first assessment response 132-1 for a range of from zero to two servings of vegetables, having predetermined associations with one or more tags 124 further associated with the dietary supplements 114 likely not consumed due to a low vegetable intake. The user can also provide a second response 132-2 for a range of from three or more servings of vegetables, having no predetermined associations a tag 124 since the user is satisfying their dietary needs with respect to servings of vegetables. If the user provides the first assessment response 132-1 indicating less than three servings of vegetables consumed each day, the recommendation generation module 120 selects, via the assessment response 132 to tag 124 lookup data structure, the one or more tags 124 further associated with the dietary supplements 114 likely not consumed due to a low vegetable intake for inclusion in a first plurality of tags 124 associated with the user. Similarly, if the user provides the second assessment response 132-2 indicating three or more servings of vegetables consumed each day, the recommendation generation module 120 identifies, via the assessment response 132 to tag 124 lookup data structure, no tags 124 for inclusion in the first plurality of tags 124 associated with the user since the user is satisfying their dietary needs with respect to servings of vegetables. In some embodiments, the dietary supplements 114 likely not consumed due to a low vegetable intake include a folic acid dietary supplement 114-1, a vitamin A dietary supplement 114-2, a vitamin C dietary supplement 114-3, a magnesium dietary supplement 114-4, a potassium dietary supplement 114-5, a carotenoid dietary supplement 114-6, a flavonoid dietary supplement 114-7, or a combination thereof.

In some embodiments, the assessment response 132 to tag 124 lookup data structure 440 identifies the first plurality of tags 124 by selecting one or more tags 124 from a pool of tags 124 associated with a respective assessment response 132. Similarly, in some embodiments, the assessment response 132 to tag 124 lookup data structure 440 identifies the first plurality of tags 124 by discarding one or more tags 124 from the pool of tags 124 associated with the respective assessment response 132 and selecting the remaining tags 124 from the pool of tags 124. In some embodiments, the pool of tags 124 associated with a respective assessment response 132 includes a predetermined pool of tags 124 or each tag 124 of the recommendation system 200. As an example, reconsider the first assessment prompt 164-1 relating to the question of a number servings of vegetables consumed each day, with assessment responses 132 available to a user in a range of from zero servings to three or more servings. If the user provides the first assessment response 132-1 indicating less than three servings of vegetables consumed each day, the recommendation generation module 120 determines one or more dietary supplements 114 likely not consumed due to a low vegetable intake and selects, via the assessment response 132 to tag 124 lookup data structure, one or more tags 124 associated with the one or more dietary supplements 114 likely not consumed from the dietary supplements of the nutrient data store 150 for inclusion in a first plurality of tags 124 associated with the user. Similarly, if the user provides the second assessment response 132-2 indicating three or more servings of vegetables consumed each day, the recommendation generation module 120 determines that no additional dietary supplements 114 are instantly needed, since the user is satisfying their dietary needs with respect to servings of vegetables, and selects, via the assessment response 132 to tag 124 lookup data structure, no tags 124 for inclusion in the first plurality of tags 124 associated with the user. By selecting one or more tags 124 from a pool of tags 124, the recommendation server system 200 can update a pool of tags 124 to reflect state-of-the-art nutritional information and research while still providing a user with a same underlying assessment 132. For instance, if research determines that a first dietary supplement 114-1 is beneficial for cardiac disease, a first tag 124-1 associated with the first dietary supplement 114-1 can be added to a pool of tags 124 further associated with an cardiovascular disease assessment prompt 164, allowing the dietary recommendations 126 to reflect the state-of-the-art.

Moreover, in some embodiments, if a respective assessment response 132 provided by a user includes data inputted by the user (e.g., text data provided by the user), the assessment response 132 to tag 124 lookup data structure 440 evaluates the respective assessment response 132 to select an appropriate tag 124 associated with the respective assessment response 132. For instance, in some embodiments, an assessment prompt 164 provides a text entry field allowing manual input of a corresponding assessment response 132. Accordingly, the assessment response 132 to tag 124 lookup data structure 440 evaluates the corresponding assessment response 132 provided by the user through the text entry field to select an appropriate tag 124 associated with the corresponding assessment response 132. As an example, consider an assessment prompt 164 pertaining to a weight of a user, with a text entry field provided that allows the user to manually input their weight as a corresponding assessment response 132. In this example, the user inputs “7.5 stone” as the corresponding assessment response 132. Accordingly, the assessment response 132 to tag 124 lookup data structure 440 evaluates the corresponding assessment response 132 and selects one or more tags 124 for inclusion in a first plurality of tags 124 associated with the user. In this example, the evaluating includes determining a body mass index (BMI) of the user, determining if the BMI of the user satisfies a threshold BMI, and in accordance with the determination that the BMI of the user satisfies the threshold BMI, selecting the one or more tags 124 for inclusion in the first plurality of tags 124. Additional details and information regarding the evaluation of selection criteria will be described in more detail infra.

Block 508. Now that an assessment response 132 to tag 124 lookup data structure 440 has generally been described in the context of block 506 which discusses the identification of such tags using a combination of the user responses and the assessment response 132 to tag 124 lookup data structure, an exemplary system of tags 124 for identifying nutritional products 112 of a dietary recommendation 126 will be described on conjunction with block 508.

The first plurality of tags 124 identified in block 506 is polled against one or more decision rules (e.g., one or more decision rules 122 of the recommendation generation module 120 of FIG. 2A). In some embodiments, the first plurality of tags 124 is polled against each decision rule 122 of the recommendation generation module 120 or, similarly, a subset of the decision rules 122. Each respective decision rule 122 is independently associated with one or more tags 124 in a second plurality of tags 124. If one or more conditions of a respective decision rule 122 is satisfied (e.g., determining the first plurality of tags 124 includes a corresponding one or more tags 124 associated with a respective decision rule 122), the respective decision rule 122 is “fired.”

The polling of the first plurality of tags 124 against the decision rules 122 causes two or more nutritional products (e.g., nutritional products 112 of the product data store 110 of FIG. 2A) to have one or more weighted or unweighted votes (e.g., points) upon polling some or all of the tags 124 of the first plurality of tags 124.

In some embodiments, the firing of a respective decision rule 122 casts a vote against, or alternatively for, one or more nutritional products 112. In some embodiments, a respective decision rule 122 casts a vote against one nutritional product 112. For instance, if a first decision rule 122-1 is fired, a vote is casted against a first nutritional product 112-1.

As an example, consider a first plurality of tags 124 including an elderberry tag 124-1, and a first decision rule 122-1 that casts a vote against an elderberry nutritional product 112-1 if the polling determines that the first plurality of tags includes the elderberry tag 124-1.

Similarly, in some embodiments, a respective decision rule 122 casts a vote against two or more nutritional products 112. As an example, if the first decision rule 122-1 is fired, a vote is cast against the first nutritional product 112-1 and a second nutritional product 112-2, such as the decision rule 122-2 of FIG. 4B. Furthermore, in some embodiments, a respective decision rule 122 cast a vote against one or more nutritional products 112 having the same category 118.

Each vote provides a point, or a tally, for one or more nutritional products 112. In some embodiments, the one or more nutritional products 112 having casted votes by a respective decision rule 122 is specified by the respective decision rule 122. For instance, the decision rule 122-2 of FIG. 4B specifying votes cast for the first nutritional product 112-1 and the second nutritional product 112-2. However, the present disclosure is not limited thereto. For instance, in some embodiments, a respective nutritional product 112 having casted votes by a respective decision rule 122 is specified by having one or more tags 124 associated with the respective nutritional products 112 further associated with the fired decision rule 122. As an example, consider a decision rule 122 associated with a caffeine tag 124, such that if the decision rule 122 is fired, each nutritional product 112 associated with a caffeine dietary supplement 114 receives a vote cast against the nutritional product 112.

In some embodiments, one or more responses 132 of a respective assessment group 168 of assessment prompts 164 for an assessment survey 162 (e.g., one or more of the above described: biometric assessment prompts 164, life-stage biometric assessment prompts 164, physiological biometric assessment prompts 164, life-style biometric assessment prompts 164, behavior biometric assessment prompts 164, etc.) either increases a contribution or decreases the contribution of the corresponding one or more nutritional products 112 by a predetermined factor. In some embodiments, the predetermined factor that either increases or decreases the contribution is unique to a respective assessment group 168, such that a first assessment group 168-1 modifies the contribution by a first factor, a second assessment group 168-2 modifies the contribution by a second factor, etc. In some embodiments, one or more of the predetermined factors modifying the contribution is a constant factor (e.g., the first factor is always an increase or decrease in the contribution of one point, the second factor is always an increase or decrease in the contribution of three points, etc.). Moreover, in some embodiments, one or more of the predetermined factors modifying the contribution is a variable factor (e.g., the first factor is an increase or decrease in contribution of in a range of from one point to three points, the second factor is always an increase or decrease in contribution of two points to six points, etc.). Furthermore, in some embodiments, a first factor is a function of a second factor. For instance, if the second factor is a function of the first factor, such that if the first factor is an increase or decrease in the contribution in a range of from one point to three points selected as two points, the second factor is always an increase or decrease in contribution of two points to six points selected at five points, then the second factor becomes an increase or decrease in contribution of ten points due to the second factor being a function of the first factor).

In some embodiments, the second plurality of tags 124 further includes one or more vitamin tags 124, one or more mineral tags 124, one or more specialty tags 124, one or more functional tags 124, or a combination thereof. For instance, in some embodiments, the second plurality of tags 124 includes each tag 124 associated with the system 100, such as each tag 124 associated with a nutritional product 112 of the product data store 110, each tag 124 associated with a decision rule 122 of the recommendation generation module 120, or a combination thereof. In some embodiments, the second plurality of tags 124 contains each vitamin tag 124 in the one or more vitamin tags 124 and each mineral tag 124 in the one or more mineral tags 124.

Generally, as the above description shows, the tags 124 are utilized by the recommendation server system 200 to translate assessment responses 132 of block 506 into one or more nutritional products 112 of a dietary recommendation 126 via the recommendation generation module 120 and the assessment responses 132 to tag 124 lookup data structure 440 in accordance with block 508.

In some embodiments, the recommendation generation module 120 stores one or more decision rules 122 for use in generating a corresponding dietary recommendation 126. The one or more decision rules 122 provide logical operations (e.g., logical operations 125-1 of FIG. 2A and as further illustrated in FIG. 4A) for the recommendation generation module 120 to translate the tags 124 identified through assessment responses into recommendations for one or more nutritional products 112. The one or more decision rules 122 poll against the plurality of tags 124 identified by the assessment responses 132 to tag 124 lookup data structure. In polling the one or more decision rules 122 against the first plurality of tags 124, if a condition of a respective decision rule 122 is satisfied by the first plurality of tags 124, the respective decision rule is considered to fire, which casts a vote for one or more nutritional products 112.

In some embodiments, the firing condition of a respective decision rule 122 is based on some Boolean combination of one or more tags 124 in the assessment responses for a subject (e.g., first decision rule 122-1 is fired when the assessment responses includes a first tag 124-1-1 and a second tag 124-1-2, first decision rule 122-1 is fired when the assessment responses includes a first tag 124-1-1 but not a second tag 124-1-2, first decision rule 122-1 is fired when the assessment responses does not include a first tag 124-1-1 but includes a second tag 124-1-2, first decision rule 122-1 is fired when the assessment responses does not include a first tag 124-1-1 and also does not include a second tag 124-1-2, first decision rule 122-1 is fired when the assessment responses includes either a first tag 124-1-1 or a second tag 124-1-2, etc.). In some embodiments, each respective decision rule 122 is associated with an independent group of one or more tags 124, such that each decision rule 122 is fired based on an independent combination of tags 124. There is no requirement, however, that each decision rule 122 have a different set of tags 124, or a different Boolean combination of tags 124. In some embodiments, at least one tag 124 is incorporated into two or more decision rules 122. Still, each respective decision rule 122 includes logical operations 125 that describe operations for polling a first plurality of tags 124 against the one or more tags 124 associated with the respective decision 122 and determining if the respective decision rule 122 is fired based on a result of the polling. In polling the first plurality of tags 124 against the one or more decision rules 122, the recommendation generation module 120 determines if the first plurality of tags 124 contains a specific tag 124 or a specific combination of tags 124 associated with each respective decision rule.

Referring briefly to FIG. 4A and FIG. 4B, and with reference to FIG. 1 through FIG. 2B, a chart 410 is provided that illustrates a variety of exemplary logical operations 125 applicable to any respective decision rule 122. In describing the following exemplary logical operations 125, a first tag 124 associated with a first decision rule 122-1 is tag 124-1-A and a second tag 124 associated with the respective decision rule 122 is tag 124-1-B. Accordingly, a first logical operation 125-1 describes an “AND” Boolean operation that requires both elements of the logical operation 125-1 to be satisfied for a respective decision rule 122 to fire. For instance, the first decision rule 122-1 will fire if a first plurality of tags 124 include the first tag 124-1-A and the second tag 124-1-B. As an example, a corresponding logical operation 125 of a respective decision rule 122-2 of FIG. 4B requires a first tag 124-1 and an eighth tag 124-8 (e.g., a folic acid tag 124-1 and a vitamin C tag 124-8) in a first plurality of tags for the decision rule 122-2 to fire.

A second logical operation 125-2 describes an “OR” Boolean operation that requires any one element of the logical operation 125-2 to be satisfied for a respective decision rule 122 to fire. For instance, the first decision rule 122-1 will fire if a first plurality of tags 124 includes either of the first tag 124-1-A or the second tag 124-1-B. As an example, a corresponding logical operation 125 of a respective decision rule 122 requires either a first tag 124-1 or an eighth tag 124-8 (e.g., a folic acid tag 124-1 or a vitamin C tag 124-8) in a first plurality of tags 124 for the respective decision rule 122 to fire.

A third logical operation 125-3 describes an “EXCLUSIVE OR” Boolean operation that requires any one element of the logical operation 125-3 to be satisfied and no other element satisfied for a respective decision rule 122 to fire. For instance, the first decision rule 122-1 will fire if a first plurality of tags 124 includes one of the first tag 124-1-A or the second tag 124-1-B, but not if neither or both of the first tag 124-1-A or the second tag 124-1-B are included in the first plurality of tags. As an example, a corresponding logical operation 125 of a respective decision rule 122 requires exclusively a first tag 124-1 or an eighth tag 124-8 (e.g., a folic acid tag 124-1 or a vitamin C tag 124-8, but not both or neither of the folic acid tag 124-1 and the vitamin C tag 124-8) in a first plurality of tags 124 for the respective decision rule 122 to fire.

A fourth logical operation 125-4 describes a singular “NOT” Boolean operation that requires absence of an element of the logical operation 125-4 to be satisfied for a respective decision rule 122 to fire. For instance, the first decision rule 122-1 will fire if a first plurality of tags 124 does not includes the first tag 124-1-A. As an example, a corresponding logical operation 125 of a respective decision rule 122 requires absence of a first tag 124-1 (e.g., a folic acid tag 124-1) in a first plurality of tags 124 for the respective decision rule 122 to fire.

A fifth logical operation 125-5 describes a plural “NOT” Boolean operation that requires both absence of a first element and presence of a second element of the logical operation 125-5 to be satisfied for a respective decision rule 122 to fire. For instance, the first decision rule 122-1 will fire if a first plurality of tags 124 includes the first tag 124-1-A but does not include the second tag 124-1-B. As an example, a corresponding logical operation 125 of a first decision rule 122-1 of FIG. 4B requires presence of a first tag 124-1 but not an eighth tag 124-8 (e.g., a folic acid tag 124-1 but not a vitamin C tag 124-8) in a first plurality of tags 124 for the first decision rule 122-1 to fire.

In some embodiments, a logical operation 125 of a respective decision rule 122 includes a combination of one or more of the above described logical operation 125. For instance, in some embodiments, a respective logical operation 125 includes one or more AND, OR, XOR, or NOT operations within the respective logical operation 125. As an example, a corresponding logical operation 125 of a third decision rule 122-3 of FIG. 4B requires presence of a tenth tag 124-10 but not either of a sixteenth tag 124-16 or a seventeenth tag 124-17 (e.g., a vitamin D tag 124-10 but not a calcium tag 124-16 or a magnesium tag 124-17) in a first plurality of tags 124 for the third decision rule 122-3 to fire.

As a further example, consider a first plurality of tags 124 containing a first tag 124-1, a second tag 124-2, and a third tag 124-3; a first decision rule 122-1 associated with the first tag 124-1 and the second tag 124-2 with a corresponding logical operation 125 requiring the first tag 124-1 and the second tag 124-2; and a second decision rule 122-2 associated with the second tag 124-2, the third tag 124-3, and a fourth tag 124-4 with a corresponding logical operation 125 requiring the second tag 124-4 or the fourth tag 124-4 but not the third tag 124-3. If this first plurality of tags 124 is polled against the first decision rule 122-1, a determination is made that the first plurality of tags 124 includes the first tag 124-1 and the second tag 124-2, therefore satisfying the logical operation 125 of the first decision rule 122-1, and the first decision rule 122-1 is fired. If this first plurality of tags 124 is polled against the second decision rule 122-2, a determination is made that the first plurality of tags 124 includes the second tag 124-2, which satisfies the logical operation 125, but also includes the third tag 124-3, which fails to satisfy the logical operation 125, therefore failing to satisfy the logical operation 125 of the second decision rule 122-2, and the second decision rule 122-2 is not fired.

While the exemplary decision rules 122 have been described above as using and polling tags 124 associated with respective decision rules 122, the present disclosure is not limited thereto. For instance, in some embodiments, a respective decision rule 122 is associated with one or more nutritional products 112. In some embodiments, one or more decision 122 is applied by the recommendation generation module 120 to refine, or filter, a subset of nutritional products 112. For instance, a first decision rule 122-1 will fire if a subset of nutritional products 112 includes a first nutritional product 112-1 and a second nutritional product 112-2. As an example, a corresponding logical operation 125 of a respective decision rule 122 requires a first nutritional product 112-1 and a second nutritional product 112-2 (e.g., a glucose nutritional product 112-1 and a metabolic boost nutritional product 112-2) in a subset of nutritional products 112 for the decision rule 122-2 to fire.

Furthermore, in some embodiments, the firing of one or more respective decision rules 122 is dependent upon a corresponding decision rule 122. For instance, in some embodiments a logical operation 125 of a respective decision rule 122 requires that a corresponding decision rule 122 fire to satisfy the logical operation 125 of the respective decision rule 122.

For instance, in some embodiments, a first decision rule 122-1 is associated with a user suitability (e.g., user viability 324 of FIG. 3) for a first macronutrient 114-1 (e.g., a first protein source) and a second decision rule 122-2 is associated with a further user suitability for specific types of the first dietary supplement 114-1 (e.g., a whey protein source, a soy protein source, a plant protein source, etc.). As such, in some embodiments, in accordance with a determination of the first decision rule 122-1 not firing if polled against the first group of tags 124, the method 500 bypasses polling the first plurality of tags 124 against the second decision rule 122-2.

In some embodiments, each time a determination is made that a respective decision rule 122 is to be fired, the respective decision rule 122 fires and casts a vote (e.g., votes 326 of FIG. 3) against one or more nutritional products 112 of the product data store 110. Each vote cast against a respective nutritional product 112 is an indication that a user could benefit from consuming the respective nutritional product 112. In this way, nutritional products 112 that receive more votes, and thus have a higher potential need or benefit for the user, are included with higher priority in dietary recommendations 126.

In some embodiments, the vote cast against the one or more nutritional products 112 by a respective decision rule 122 is a weighted vote, as opposed to an unweighted vote. For instance, in some embodiments, an unweighted vote accounts for one point against a respective nutritional product 112 and a weighted vote count for less than or greater than one point (e.g., does not equal one) against the respective nutritional product 112, such that a contribution of the corresponding one or more nutritional products 112 in generating a dietary recommendation 126 is modified by the weight of such votes. In some embodiments, “1,” also referred to as “unity” herein is considered an unweighted vote and a weighted vote is the algebraic combination of an unweighted vote (e.g., “1”) and a coefficient, where the coefficient is some number other than “1.” In some embodiments the coefficient is a positive number between 0 and 1 (e.g., 0.5, 0.8, etc.). In some embodiments the coefficient is a positive number between 1 and 100 (e.g., 1.5, 2.0, 3.3, etc.). As used herein, this coefficient is referred to as a weight. Weighted voting is used to reflect the fact that some tags, and the decision rules they fire when such tags are present (or absent), may have different importance than other tags, and the decision rules they fire when such tags are present (or absent).

In some embodiments, a weight of a vote is determined based on a need to satisfy a nutritional product selection criteria (e.g., nutrition criterion 342 of FIG. 3), such as a nutrient metric 154 of the nutrient data store 150. For instance, in some embodiments, a dietary recommendation 126 provided to a respective user must satisfy a threshold nutrient metric 154 for a first dietary supplement 112-1. To ensure that the threshold nutrient metric 154 is satisfied, given a first plurality of tags 124 identified by the assessment response 132 to tag 124 lookup data structure, a first decision rule 122-1 requires that the first plurality of tags 124 include a first tag 124-1 and a certain number of second tags 124. Accordingly, if the first plurality of tags 124 includes the first tag 124-1 and a certain threshold predetermined number of second tags 124, a weighted vote is cast by the first decision rule 122-1 for a first nutritional product 112-1. If the first plurality of tags 124 includes the first tag and a number of second tags less than the threshold number of second tags, a weighted vote is cast by the second decision rule 122-2 for a second nutritional product 112-2. Similarly, in some embodiments, if the first plurality of tags 124 includes the first tag and a number of second tags less than a threshold predetermined number of second tags, a weighted vote is cast by the second decision rule 122-2 for the first nutritional product, with the weighted vote cast by the second decision rule 122-2 being different than the weighted vote cast by the first decision rule 122-1. As an example, consider a first nutritional product 112-1 including 450 mg of a calcium dietary supplement 114-1, while a second nutritional product 112-2 includes 1,000 mg of the calcium dietary supplement 114-1. To ensure an equivalent calcium dietary recommendation for each user, if a first plurality of tags 124 identified by the assessment response 132 to tag 124 lookup data structure 440 includes a first calcium tag 124-1 and less than five total tags 124 associated with a micronutrient dietary supplement 114, a weighted vote is cast, by the decision rule, for the second nutritional product 112-2 because it has a higher dosage of calcium.

In some embodiments, a weight of a vote cast by a respective decision rule 122 is determined by a corresponding assessment response 132 provided by the user, such that the user provided assessment responses 132 affect the weight of various casted votes. For instance, in some embodiments, if the corresponding assessment response 132 provides an indication of a beneficial or adverse reaction to one or more dietary supplements 114 or one or more nutritional products 112, the respective decision rule 122 casts weighted votes that reflect the beneficial or adverse nature of the reaction. As an example, consider an assessment response 132-1 for a first user 10-1 indicating that the first user has an adverse reaction to a caffeine dietary supplement 114-1, and an assessment response 132-1 for a second user 10-2 indicating that the second user has no adverse reactions to a caffeine dietary supplement 114-1. Accordingly, upon firing, a corresponding decision rule 122 will cast a weighted vote that decreases a contribution of one or more nutritional products 112 that include the caffeine dietary supplement 114-1 for the first user 10-1, while casting an unweighted or upweighted vote for the one or more nutritional products 112 that include the caffeine dietary supplement 114-1 for the second user 10-2.

Block 510. The method 500 identifies a subset of the available nutritional products 112 (e.g., a subset of one or more nutritional products 112 from the collective nutritional products 112 of the product data store 110 of FIG. 2A) in accordance with a result of the polling described above for block 508. In some embodiments, the subset of nutritional products 112 is identified on a basis of the corresponding weighted or unweighted votes respectively received by each of one or more nutritional products 112. This basis of the weighted or unweighted votes is conducted as a tally of the votes, such as a summation of the points contributed against a respective nutritional product 112. Accordingly, in some embodiments, the subset of the nutritional products 112 consists of those nutritional products 112 that each received a sufficient number of weighted or unweighted votes by the polling.

In some embodiments, the number of weighted or unweighted votes is evaluated to determine if the number weighted or unweighted votes satisfies one or more nutritional product selection criterion (e.g., nutrient metric 154 of a respective dietary supplement reference data 15 of FIG. 2B). In some embodiments, the nutritional product selection criterion described by a respective nutrient metric 154 is satisfied in accordance with a determination that a respective nutritional product 112 obtains a threshold number of votes, such as greater than or equal to one vote. In some embodiments, the nutritional product selection criterion described by a respective nutrient metric 154 is satisfied in accordance with a determination that a respective nutritional product 112 satisfies a threshold ranking, in terms of votes, relative to all the other nutritional products under consideration. In some embodiments, the threshold ranking is satisfied if the number of obtained votes for the respective nutritional product 112 places the respective nutritional product 112 within a specified ranking if the subset of nutritional products 112 is ordered by number of obtained votes (e.g., within the top five nutritional products 112 in the subset of nutritional products 112 according to a number of obtained votes). For instance, in some such embodiments, the threshold ranking is satisfied if the number of obtained votes for the respective nutritional product 112 places the respective nutritional product 112 in the top 10, 9, 8, 7, 6, 5, 4, 3, or 2 products, in terms of votes.

In some embodiments, the subset of nutritional products 112 is evaluated to determine if a threshold period nutritional limit (e.g., a respective nutrient metric 154) is satisfied by the subset of nutritional products 112. Depending on a result of the determination, one or more nutritional products 112 is substituted or included in the subset of nutritional products 112 to satisfy the threshold period nutritional limit. For instance, consider a first subset of nutritional products 112 that includes four nutritional products 112, each of which includes a first dietary supplement 114-1. This first subset of nutritional products 112 is evaluated to determine if a threshold period nutritional limit is satisfied. Here, the threshold period nutritional limit is a maximum dosage of the first dietary supplement 114-1, which is exceeded by the first subset of nutritional products 112. As such, one or more nutritional products of the first subset of nutritional 112 is omitted from the first subset of nutritional products 112 or substituted for a different nutritional product in order to ensure that the threshold period nutritional limit is satisfied.

In some embodiments, in accordance with a determination that the subset of nutritional products 112 satisfies a threshold score of unique tags 124, a first nutritional product 112-1 is include in the subset of nutritional products 112 includes. In some embodiments, the threshold score consists of five or more unique tags 124. Accordingly, when the subset of nutritional products exceeds the threshold score of unique tags 124, the nutritional products 112 in the subset of nutritional products 112 is refined. As an example, and with reference to FIG. 4B, decision rule 122-5 requires that if the subset of nutritional products 112 includes five or more vitamin and/or mineral tags 124, a seventh nutritional product 112-7, which is a multi-vitamin, is substituted into the subset of nutritional products 112.

In some embodiments, each nutritional product 112 of the product data store 110 is associated with a respective classification 118. Accordingly, the method 500 includes assigning a respective classification 118 from the available classifications 118 to the subset of nutritional products 112. In some embodiments, each classification 118 associated with a respective nutritional product 112 in the subset of nutritional products 112 is evaluated to determine which classification 118 is a statistical mode of the collective classifications 118 of the subset of nutritional products 112, which is a most common classification 118 in the collective classifications 118 of the subset of nutritional products 112. The classification 118 that is the statistical mode is then further assigned to the subset of nutritional products 112. For instance, if a subset of nutritional products 112 includes a first nutritional product 112-1 assigned to a first classification 118-1, a second nutritional product 112-2 assigned to the first classification 118-1, and a third nutritional product 112-3 assigned to a second classification 118-2; the subset of nutritional products 112 is assigned to the first classification 118-1 since the first classification 118-1 is the mode classification 118 to the subset of nutritional products 112.

In some embodiments, each nutritional product 112 in the subset of nutritional products 112 is associated with the respective classification 118 that is assigned to the subset of nutritional products 112. Accordingly, the classifications 118 provide bundles of related nutritional products 112 that are commonly provided to various user demographics or provide a common health related benefit. For instance, in some embodiments, a first classification 118-1 provides a first combination of one or more nutritional products 112 for vegan pregnant women, a second classification 118-2 provides a second combination of one or more nutritional products 112 for relatively inactive and overweight men, a third classification 118-3 provides a third combination of one or more nutritional products that each include lactose, etc.

In some embodiments, the method 500 polls the first plurality of tags 124 to determine if one or more specific tags 124 is, or similarly is not, contained in the first plurality of tags 124. In some embodiments, the polling of the first plurality of tags 124 determines if a first tag 124-1 is contained in the first plurality of tags 124. In some embodiments, the polling of the first plurality of tags 124 determines if a first tag 124-1 and a second tag 124-2 are contained in the first plurality of tags 124. As such, in accordance with a determination that the first plurality of tags 124 includes a first tag 124-1 and a second tag 124-1, a first product 112-1 is included in the subset of nutritional products 112-1. Furthermore, in some embodiments, the polling of the first plurality of tags 124 determines if a first tag 124-1 but not a second tag 124-2 is contained in the first plurality of tags 124. This inclusion of the first product 112-1 into the subset of nutritional products 112 based on the aforementioned determination of contained tags 124 either substitutes the first product 112-1 for a second product 112-2 in the subset of nutritional products 112 or adds the first product 112-1 to the subset of nutritional products 112. In some embodiments, the first product 112-1 is added to the subset of nutritional products 112 if the first product 112-1 contains one or more specialty dietary supplements 114 and/or one or more functional dietary supplements 114. Furthermore, in some embodiments, the first product 112-1 is substituted into the subset of nutritional products 112 if the first product 112-1 contains one or more vitamin dietary supplements 114, one or more mineral dietary supplements 114, one or more functional dietary supplements 114, or a combination thereof. As an example, if the first plurality of tags 124 includes a vitamin B tag 124-1 and a vitamin C tag 124-2, the subset of nutritional products includes a first nutritional product 112-1 including a vitamin B dietary supplement 114-1 and a vitamin C supplement 114-2. As a further example, if the first plurality of tags 124 includes a vitamin B6 tag 124-3 and a vitamin B12 tag 124-4, the subset of nutritional products removes a second nutritional product 112-2 (a vitamin B type dietary supplement 114-1) to ensure the subset of nutritional products is not exceeding a vitamin B nutrient metric 154, such as a periodic nutritional limit.

Upon polling the decision rules 122 against the first plurality of tags 124 identified by the assessment response 132 to tag 124 lookup data structure, a subset of nutritional products 112 is identified based on the votes received against each nutritional product 112. In some embodiments, the subset of nutritional products 112 is identified by selecting each nutritional product that satisfies a nutritional product selection criterion. In some embodiments, the nutritional product selection criterion includes a threshold number of votes. Satisfying the threshold number of votes includes obtaining a predetermined number of votes, such as obtaining greater than or equal to ten votes, or obtaining more votes relative to other nutritional products 112 (e.g., a higher rank), such as obtaining a number of votes within a top five number of votes for any nutritional product 112.

In some embodiments, the recommendation generation module 120 applies a machine learning algorithm that ranks two or more potential combinations of one or more nutritional products 112 in generating a dietary recommendation 126. In some embodiments, this ranking forms a hierarchy of nutritional products 112 based on the votes cast against the nutritional products. The machine learning algorithm includes linear regression models, logistic regression models, linear discriminant analysis models, a classification and regression tree models, a naïve Bayes models, a K-Nearest Neighbor models, an artificial neural network models (e.g., learning vector quantization), a support vector models, a random forest models, or a combination thereof.

In some embodiments, the recommendation generation module 120 exchanges data with other modules (e.g., the product data store 110, user response data store 130, etc.) of the recommendation system 200 in order to incorporate various data received therefrom in the generation of a dietary recommendation 126. For instance, in some embodiments, the recommendation generation module 120 communicates with the product data store 110 in order to determine an availability of various nutritional products 112. Moreover, this determination is factored into generating of the dietary recommendation 126. In some embodiments, the recommendation generation module 120 is in communication with the user response data store 130. This communication with the user response data store 130 allows for the recommendation generation module 120 to account for various user preferences and conditions (304 through 308 of FIG. 3).

In some embodiments, the identifying the subset of nutritional products 112 further includes filtering the subset of nutritional products 112 based on one or more tags 124 associated with a respective assessment response 132 provided by the user. For instance, in some embodiments, if the first plurality of tags 124 includes a first tag 124-1 and a second tag 124-2, each of which is unassociated with a respective dietary supplement 114, the inclusion of the first tag 124-1 and the second tag 124-2 signals for a specific filtering condition, such as omitting one or more nutritional products 112 containing a corresponding dietary supplement 114 or including a corresponding nutritional product 112.

Block 512. Referring to FIG. 5B, in some embodiments, the method 500 includes filtering (e.g., constraint filter 340 of FIG. 3) the subset of nutritional products 112 against one or more periodic nutritional limits (e.g., one or more nutritional metrics 154 of FIG. 2B; nutritional limit of FIG. 4C). In some embodiments, each respective periodic nutritional limit metric 154 specifies a corresponding maximum dosage of a corresponding dietary supplement, such as a corresponding dietary supplement 114 or a corresponding product 112. Furthermore, in some embodiments, each respective period nutritional limit metric is a function of time, such that the nutritional limit varies depending on the period of time being considered. For instance, in some embodiments, a respective periodic nutritional limit is determined according to a daily period of time, a weekly period of time, a yearly period of time, and the like. In some embodiments, the maximum dosage is a dosage of a respective dietary supplement 114 that is safe for consumption in a period of time. In some embodiments, the maximum dosage given is a function of one or more physiological characteristics of the user, such as a characteristic provided in an assessment response 132 (e.g., weight, age, sex, pregnant, not pregnant, diabetic, etc.)

Referring to FIG. 4C, in some embodiments, the filtering independently sums, for each respective dietary supplement 114 associated with a respective periodic nutritional limit and included in a nutritional product 112 of the subset of nutritional products 112, the corresponding amount of respective dietary supplement 114 in either the subset of nutritional products 112 and/or each individual nutritional product. For instance, the chart 430 of FIG. 4C determines an amount of each respective dietary supplement 114 collectively contained in the subset of nutritional products 112 (e.g., dietary supplement 114-1 through 114-6 of nutritional product 112-1 though nutritional product 112-6).

In some embodiments, the filtering removes from the subset of nutritional products 112 one or more doses of one or more nutritional products 112 in accordance with a determination that one or more periodic nutritional limits is determined by the filtering to have been exceeded. This removal of a dosage of a nutritional product 112 prevents the one or more periodic nutritional limits from being exceeded. For instance, as illustrated in FIG. 4C, the subset of nutritional products 112 includes an excess of a fifth dietary supplement 114-5 of 600 mg. As such, the method 500 removes two dosages of a first nutritional product 112-1, lowering the total amount of the fifth dietary supplement 114-5 to 600 mg (e.g., 500 mg from a first dosage and 100 mg from an anticipated daily value), which satisfies the nutritional limit of 1,000 mg. In some embodiments, removing one or more dosages of a respective nutritional product removes the entire nutritional product 112 from the dietary recommendation 126. In some embodiments, in accordance with a determination that one or more periodic nutritional limits is exceeded, the filtering substitutes at least a first nutritional product 112-1 in the subset of nutritional products 112-1 for a second nutritional product 112-2, such that the previously exceeded nutritional limit is no longer exceeded.

In some embodiments, the filtering evaluates an amount of each respective dietary supplement 114 a user is anticipated to consume on from their standard diet (e.g., daily value of FIG. 4C). In some embodiments, the anticipated consumption of a respective dietary supplement 114 is determined through one or more assessment responses 132 provided by a user (e.g., a health goal of the user, a current diet of the user) and/or one or more physiological characteristics of the user. For instance, in some embodiments, the filtering includes determining a body mass index (BMI) of the user. Accordingly, the nutritional product selection criterion accounts the filtering is based on includes the BMI of the user. This allows the dietary recommendation 126 to be specifically tailored to the specifications of the user, rather than being based on a standard conventional diet that might provide less than optimal health benefits for the user. In some embodiments, the BMI of the user is based on one or more assessment responses 132 obtained from the user, such as a weight of the user and a height of the user. In some embodiments, the BMI, or similarly the component weight and/or height of the user, is derived from a medical record associated with the user or from data previously obtained from the user (e.g., user response data store 130 of FIG. 2A).

In some embodiments, the filtering further includes determining a target caloric intake of the user (e.g., nutrition limits 344 of FIG. 3). Accordingly, the nutritional product selection criterion accounts for the target caloric intake of the user, which provides a more accurate nutritional limit for the filtering. In some embodiments, the target caloric intake of the user is based on one or more assessment responses 132. For instance, in some embodiments, the one or more assessment responses 132 obtained from the user include data related to a weight of the user, a height of the user, a sex of the user, an age of the user, a level of physical activity endured by the user, or a combination thereof. For instance, in some embodiments, the target caloric intake of a respective user is a function of a BMR of the respective user and an activity factor variable (e.g., an activity level of the respective user determined through one or more responses 132). As an example, if a user is male, the target caloric intake of the user is a function of a weight of the user times a first constant, in additional to a height of the user times a second constant, in further addition to an age of the user times a third constant, and in addition to a fourth constant. If the user is female, the target caloric intake of the user is a function of the weight of the user times the first constant, in additional to the height of the user times the second constant, in further addition to the age of the user times the third constant, and in addition to a fifth constant.

Moreover, in some embodiments, the filtering further evaluates a possibility of exceeding a no significant risk level (NSRL) or a maximum allowable dose level (MADL) for cancer-causing chemical and chemicals known to cause re-productivity toxicity, such as one or more specific dietary supplements 114 associated with a heavy metal.

In some embodiments, the filtering includes evaluating each nutritional product 112 in the subset of the nutritional products 112 against a dimensional constraint associated with one or more of the nutritional products 112 (e.g., product data 116-1 of FIG. 2A) in the subset of nutritional products 112. For instance, in some embodiments, the dimensional constraint includes a maximum number of dosages (e.g., dosages of FIG. 4C) contained in a provision 128 of a dietary recommendation 126, a collective mass of the subset of nutritional products 112, and the like. In some embodiments, a provision 128 of a dietary recommendation 126 is limited to a predetermined number of total dosages among a subset of nutritional products 112, such as six total dosages. In some such embodiments, if a respective nutritional product 112 requires more than one dosage for an effective dietary recommendation 126, the method 500 evaluates each nutritional product 112 to determine if dosages of the respective nutritional product 112 are removed from the dietary recommendation 126. In some embodiments, this evaluating is provided manually by the user (e.g., manual input 352 of FIG. 3), such as a user providing a personal limit of dosages or shipping size of a provision 128.

In some embodiments, the filtering the nutritional product 112 includes determining an availability of each nutritional product 112 (e.g., in the subset of nutritional products 112). In some embodiments, the availability is based on an inventory of the respective nutritional product and/or a geographic restriction associated with the respective nutritional product or the physical location associated with the subject.

Block 514. Using the subset of nutritional products 112, the method 500 provides a dietary recommendation 126 to the user (e.g., provide recommendation 350 of FIG. 3). In some embodiments, the dietary recommendation 126 is provided to the user after filtering the subset of nutritional products 112 (e.g., after block 512 of FIG. 5B). By way of example, referring briefly to FIGS. 6E and 6F, a dietary recommendation 126 is presented to a user, which includes a first dietary recommendation 126-1 of a first subset of nutritional products 112 and a second dietary recommendation 126-2 of a second subset of nutritional products 112 other than the first subset of nutritional productions 112. Accordingly, the user is provided with a choice between the first subset of nutritional products 112 and the second dietary recommendation 126-2 based on a plurality of assessment responses 132 provided by the user, which provide the same, or substantially the same, nutritional benefit for the user.

In some embodiments, the dietary recommendation 126 is provided to the user as the subset of nutritional products 112, such as a listing of each nutritional product 112 in the subset of nutritional products 112. In some embodiments, the dietary recommendation 126 is provided to the user in the form a report, such as an electronic report (e.g., report 600 of FIGS. 6E through 6H). In some embodiments, the report is provided with a provision 128 of the dietary recommendation 126. For instance, referring briefly to FIG. 6E, a report of a second dietary recommendation 126-2 is presented to a subject. The report includes a listing of one or more nutritional products 112 in a subset of nutritional products 112 of the second dietary recommendation 126-2. Here, the report includes a mechanism 138 that allows a user to modify an inclusion of a respective nutritional products 112 in a provision of the second dietary recommendation 126. Moreover, the listing of each nutritional product 112 in the subset of nutritional products 112 of the second dietary recommendation 126-2 includes a description of each tag 124 associated with a corresponding nutritional product. Furthermore, in some embodiments, the listing of each nutritional product 112 in the subset of nutritional products 112 of the second dietary recommendation 126-2 includes a description of one or more assessment responses 132 provided by the user that formed a basis for inclusion of the corresponding nutritional product 112. Additionally, referring briefly to FIGS. 6G and 6H, in some embodiments, the report includes a listing of the one or more corresponding dietary supplements 114 associated with a respective nutritional product 112 in the subset of nutritional products of a dietary recommendation 126 of the subject.

In some embodiments, the dietary recommendation 126 includes one or more descriptive portions (e.g., descriptions). Each descriptive portion provides a variety of information related to an opportunity for the user to improve a health-related aspect. In some embodiments, the improved health related aspect is provided by consuming one or more nutritional products 112 the subset of nutritional products 112. In some embodiments, the descriptive portion includes a warning related to one or more nutritional products 112 in the dietary recommendation 126. As an example, in some embodiments, a respective nutritional product 112 is required to satisfy a dietary need of the user but the respective nutritional product 112 does not conform to a dietary preference of the user. As such, the user is provided with a warning regarding the lack of conformity (e.g., a warning of “nutritional product 112-2 includes a gelatin capsule, which does not conform to your vegan diet.”). As another example, in some embodiments, a respective nutritional product 112 is provided to in a dietary recommendation 126 to fulfill a dietary need of the user. However, the user is provided with a warning through the dietary recommendation 126 for a consideration to consult a medical practitioner regarding the dietary need of the user (e.g., a warning of “nutritional product 112-3 is recommended since you feel tired all the time, consider consulting your physician.”). In some embodiments, the descriptive portion includes a description of information for a motivation (e.g., to address a dietary supplement 114 deficiency, for a health goal, etc.) to include one or more nutritional products 112 in the dietary recommendation 126.

In some embodiments, the descriptive portion includes a description of information for a reasoning to include one or more nutritional products 112 in the dietary recommendation 126. This reasoning to include one or more nutritional products 112 includes a reasoning for recommending a respective nutritional product 112 and/or a reasoning as to why the respective nutritional product 112 is included in a dietary recommendation. For instance, in some embodiments, one or more nutritional products 112 is included in the dietary recommendation 126 due to an age of the user (e.g., to address an age-based nutritional need of the user), due to a dietary deficiency of the user (e.g., to address a need for a dietary supplement 114), due to a gender specific need of the user (e.g., to address a post-natal need), due to a health condition of the user (e.g., to address weight control and/or blood sugar management of the user), due to a life style of the user (e.g., to address a fitness need of the user, to address a dietary preference of the user, etc.), and the like. As an example, a first assessment prompt 164-1 is provided to a first user 10-1 and a second user 10-2. Responsive to the first assessment prompt 164-1, the first user 10-1 provides a first assessment response 132-1 indicating that the first user 10-1 has trouble staying asleep, and the second user 10-2 provides a second assessment response 132-2 indicating that the second user 10-2 does not get enough sleep. Accordingly, the method 500 determines that each respective dietary recommendation 126 provided to the first user 10-1 and the second user 10-2 includes a first nutritional product 112-1. However, the description of the respective dietary recommendation 126 provided to the first user 10-1 includes information that the first nutritional product 112-1 is included for addressing the sleep needs of the user and since the user is having trouble staying asleep, and the description of the same dietary recommendation 126 provided to the second user 10-2 includes information that the first nutritional product 112-1 is included for addressing the sleep needs since the second user 10-21 does not get enough sleep. Thus, each user is provided different information as to why a nutritional product 112 is included in their respective dietary recommendation 126.

In some embodiments, the descriptive portion includes a description of information for a health claim (e.g., this nutritional product 112 enhances bone strength, etc.) include one or more nutritional products 112 in the dietary recommendation 126.

In some embodiments, the dietary recommendation 126 includes a dosage regimen for one or more nutritional products 112 of the dietary recommendation 126. In some embodiments, the dosage regimen is determined in accordance with one or more physiological characteristics of the user, such as user height, weight, gender, dietary preference, and the like. For instance, in some embodiments, the method 500 determines, through evaluating the one or more physiological characteristics, that the user requires a substantially different diet than a standard diet (e.g., a standard recommended diet based on a 2,000 Caloric intake). Accordingly, the dosage regimen provided through the dietary recommendation 126 considers this substantial difference in diet of the user.

In some embodiments, the dietary recommendation 126 provides the user an opportunity to modify one or more nutritional products 112 of the dietary recommendation 126 (e.g., manual input 352 of FIG. 3). For instance, in some embodiments, the user is provided an opportunity to substitute one product 112-1 for another product 112-2 in the dietary recommendation 126, or similarly, including the first product 112-1 with the second product 112-2 in the dietary recommendation 126. In some embodiments, the nutritional products 112 available to the user for inclusion and/or substitution with the dietary recommendation 126 are provided only if the further inclusion and/or substitution of a respective nutritional products 112 does not significantly alter nutritional benefits provided by the dietary recommendation 126. For instance, if a dietary recommendation 126 includes a first product 112-1 the dietary recommendation can include a second product 112-2 and a third product 112-3 as an available substitute for the first product 112-1 if the second product 112-2 and a third product 112-3 provide a similar nutritional benefit. As such, the user can substitute the second product 112-2 or the third product 112-3 for the first product 112-1 without affecting the nutritional benefits provided by the dietary recommendation.

In some embodiments, the method 500 further includes shipping a provision 128 of the dietary recommendation 126 to a physical location associated with the user. In some embodiments, the physical location is included in an assessment response 132 and/or stored in the user data store 130.

### EXAMPLE 1: PROVIDING A DIETARY RECOMMENDATION FOR A FIRST USER

A first user is provided an assessment survey 162 through a first user device 10-1. The assessment survey 162 includes a variety of assessment prompts 164. The first user provides assessment responses 132 for some or all of the assessment prompts 164 provided by the assessment survey 162.

The assessment responses 132 provided by the first user include: a height of the first user of five foot, three inches; a weight of the first user of 105 pounds (lbs); an age of the first user of forty years of age; a gender of the first user of female; the first user has a family history of high cholesterol, heart disease, and high blood pressure; a previous recommendation from a medical practitioner for a dietary supplement 114 including omega-3 fatty acids, fruits, vegetables, or fish in the diet of the first user; a stress level of the first user includes experiencing stress; a cognitive level of the first user includes having a difficult time recalling memory; a computer use level of first user include three hours per day; a health goal of the first user for maximizing endurance performance in physical activities; and overall goals of the first user for improved stress management, improved energy, and improved immunity.

Accordingly, the above assessment responses 132 are obtained by the recommendation server system 200 from the first user device 10-1 (e.g., input data 304 of FIG. 3). These assessment responses 132 are evaluated using an assessment response 132 to tag 124 lookup data structure 440 (e.g., product data store 110, nutrient data store 150, assessment store 160, or a combination thereof of FIG. 2A). A set of tags 124 is identified through the assessment response 132 to tag 124 lookup data structure, which is associated with assessment responses 132 provided by the first user.

In some embodiments, the assessment responses 132 provided by the first user provide the set of tags 124 including: a folic acid tag 124-1 including a folic acid tag and a folid acid brain tag, a vitamin A tag 124-2, a vitamin B12 tag 124-3 including a B12 tag, a B12 plus tag, and a B12 energy tag, a vitamin C tag 124-4 including a vitamin C tag and a vitamin C immunity tag, a vitamin D tag 124-5, a vitamin E tag 124-6, a vitamin K tag 124-7, a calcium tag 124-8 including a calcium plus tag and a calcium sleep tag, a magnesium tag 124-9 including a magnesium blood pressure tag and a magnesium plus tag, a potassium tag 124-10, and a zinc tag 124-11 including a zinc immunity tag. In some embodiments, the assessment responses 132 provided by the first user provide the set of tags 124 further including: an ashwaganda tag 124-12, a carotenoids tag 124-13, an echinacea tag 124-14, an elderberry tag 124-15, a flavonoids tag 124-16, a larch tree tag 124-16, a L-Theanine tag 124-17, a L-Tyrosine tag 124-18, an omega-3 fatty acid tag 124-19 including an omega-3 fatty acid tag and a 2,000 mg omega-3 fatty acid tag, a probiotics tag 124-20, and a procyanidins tag 124-21.

In some embodiments, the specialty tags 124 (e.g., the ashwaganda tag 124-12, the carotenoids tag 124-13, the echinacea tag 124-14, the elderberry tag 124-15, the flavonoids tag 124-16, the larch tree tag 124-16, the L-Theanine tag 124-17, the L-Tyrosine tag 124-18, the omega-3 fatty acid tag 124-19 including the omega-3 fatty acid tag and the 2,000 mg omega-3 fatty acid tag, the probiotics tag 124-20, and the procyanidins tag 124-21) are not considered in determining a number of tags 124 in the set of tags 124. Furthermore, in some embodiments, the above tags 124 including specific dietary supplement tags are not further considered in determining the number of tags 124 in the first plurality of tags 124 (e.g., the omega-3 fatty acid tag 124-19 including the omega-3 fatty acid tag and the 2,000 mg omega-3 fatty acid tag collectively account for one tag rather than individually accounting for three tags).

Accordingly, the first plurality of tags 124 for the first user 10-1 is determined to satisfy a threshold number of unique tags 124. In the instant embodiment, ten total unique tags 124 associated with either a vitamin dietary supplement 114 or a mineral dietary supplement 114 are included in the first plurality of tags 124, which is greater than a predetermined threshold number of unique tags 124 of greater than or equal to five.

As such, a dietary recommendation 126 is generated and provided to the first user (e.g., provide recommendation 350 of FIG. 3; block 514 of FIG. 5B). Since the set of tags 124 associated with the user responses 132 satisfied the threshold value (e.g., greater than or equal to five unique tags 124), one or more nutritional products 112 in the dietary recommendation 126 are substituted for another nutritional product 112 (e.g., a first product 112-1, a second product 112-2, and a third product 112-3 are consolidated (e.g., substituted) for a fourth product 112-4 that provides similar nutritional benefits as the collective first through third products 112, such as a replacing a number of nutritional products 112 with a multivitamin product 112). In the instant embodiments, one or more the one or more nutritional products 112 substituted in the dietary recommendation 126 are substituted in place a multi-vitamin nutritional product 112, which consolidates a size of a provision 128 of the dietary recommendation 126.

Accordingly, the dietary recommendation 126 for the first user includes a first product 112-1 (e.g., Vita-Lea™ Women), a second product 112-2 (e.g., two dosages of B-complex), and a third product 112-3 (e.g., Sustained Release Vita-C®). In some embodiments, the dietary recommendation 126 further includes one or more specialty products 112 (e.g., products 112 associated with a specialty tag 124) including a fourth product 112-4 (e.g., CAROTOMAX®), a fifth product 112-5 (e.g., FLAVOMAX®), a sixth product 112-6 (e.g., MindWorks®), a seventh product 112-7 (e.g., OMEGAGUARD® Plus), an eighth product 112-8 (e.g., OPTIFLORA® DI), and an eight product 112-8 (e.g., Stress Relief Complex). In some embodiments, the one or more specialty products 112 are provided to the first user as optional inclusion within a provision 128 of the dietary recommendation 126. For instance, the one or more specialty products 112 are provided in a separate hierarchy of nutritional products 112. As such, the first user is provided an opportunity to include or omit each of the one or more specialty products 112 in the provision 128 (e.g., manual input 352 of FIG. 3).

In some embodiments, the dietary recommendation 126 for the first user 10-1 is refined prior to providing the dietary recommendation 126 to the first user 10-1. In some embodiments, the nutritional products 112 of the dietary recommendation 126 are filtered (e.g., constraint filter 340 of FIG. 3; filtering 512 of FIG. 5B); to remove redundant dietary supplement 114 stacking (e.g., nutrition limits 344 of FIG. 3), such as multiple sources of a specific dietary supplement 114.

For instance, B-Complex nutritional product 112-8 and MindWorks® nutritional product 112-6 both include vitamin B dietary supplement 114. If a user receives a B-Complex nutritional product 112-8 (e.g., one or two dosages) and a MindWorks® nutritional product 112-6 in the dietary recommendation 126, the B-Complex nutritional product 112-8 is substituted, or similarly removed, from the dietary recommendation 126 to prevent reductant stacking of the vitamin B dietary supplement 114, preventing the user from consuming an excess amount of the vitamin B dietary supplement 114. As such, the dietary recommendation 126 provided to the first user includes the MindWorks® recommendation only rather than both the B-Complex and the MindWorks® recommendation.

### EXAMPLE 2: PROVIDING A DIETARY RECOMMENDATION FOR A SECOND USER

A second user (e.g., a user associated with a second user device 10-2, a second user associated with a first user device 10-1, etc.) is provided an assessment survey 162. The assessment survey 162 includes a variety of assessment prompts 164. The second user provides assessment responses 132 for some or all of the assessment prompts 164 provided by the assessment survey 162.

The assessment responses 132 provided by the first user include: a height of the second user of five foot, three inches; a weight of the first user of 105 lbs; an age of the first user of forty years of age, a sex of the user of female, a familial history of the user of: high cholesterol, heart disease, and high blood pressure, a medical practitioner recommendation of the user of increased in: omega-3s, fruits, vegetables, or fish, a stress level of the user of experiences stress, a cognitive level of the user of having difficulty remembering, a computer use level of the user of more than three hours per day, a health goal of the user to maximize endurance performance, and a ranking of health goals of a high priority for stress management (e.g., a top goal), a medium priority of energy, and a low priority of improved sleep. The assessment responses 132 further indicate that the second user consumes one or more dietary supplements 114 to make up for dietary deficiencies. In some embodiments, the one or more dietary supplements 114 the second user is consuming include a nutritional product 112 of the product data store 110 of FIG. 2A.

Accordingly, these assessment responses 132 from the second user are used with an assessment response 132 to tag 124 lookup structure (e.g., product data store 110, user response data store 130, nutrient data store 150, or a combination thereof) to select a corresponding set of tags 124 for each respective assessment response 132. Collectively, the sets of tags 124 from each respective assessment response 132 form a first plurality of tags 124 associated with the second user identified by the assessment response 132 to tag 124 lookup structure. This first plurality of tags 124 is polled against one or more decision rules 122, which collectively cast votes against a number of nutritional products 112.

The nutritional products 112 are ranked by votes (e.g., ranked using ranker module 140 of FIG. 2B, ranked using ranking engine 320 of FIG. 3, etc.) to determine a hierarchy of nutritional products 112 of the product data store 110. Here, consider the nutritional products 112 that received votes to include: a first nutritional product 112-1 that addresses one or more dietary deficiencies and a first health problem (e.g., the familial history of heart disease) of the second user; a second nutritional product 112-2 that addresses one or more dietary deficiencies of the second user; a third nutritional product 112-3 that addresses one or more dietary deficiencies of the second user, a fourth nutritional product 112-4 that addresses a second health problem (e.g., the experiencing stress) of the second user, a fifth nutritional product 112-5 that addresses a third health problem (e.g., the difficulty remembering) of the second user, a sixth nutritional product 112-6 that addresses one or more dietary deficiencies of the second user; a seventh nutritional product 112-7 that addresses one or more dietary deficiencies of the second user; and an eighth nutritional product 112-8 that addresses one or more dietary deficiencies of the second user. Each of the aforementioned respective nutritional products 112 has a corresponding set of tags 124 that collectively form the first plurality of tags 124.

Accordingly, a count of a number of unique tags 124 in the first plurality of tags 124 is determined. In this example, the count of the number of unique tags 124 includes a summation of a number of unique mineral tags 124 and a number of unique vitamin tags 124 (e.g., the count does not include specialty tags 124 and/or functional tags 124). As such, the recommendation server system 200 determines that the number of unique tags 124 the first plurality of tags 124 is greater than or equal to a predetermined number of five. Since the number of unique tags satisfies the threshold number of five, the recommendation server system 200 substitutes a ninth nutritional product 112-9 (e.g., a multi-vitamin nutritional product 112-9) for one or more of the lowest ranked nutritional products in the hierarchy of nutritional products 112. As such, the recommendation server system 200 substitutes the ninth nutritional product 112-9 for the sixth nutritional product 112-6, the seventh nutritional product 112-7, and the eighth nutritional product 112-8. This substitution of the ninth product 112-9 limits a total number of nutritional products 112 in a dietary recommendation 126 and/or prevents redundant stacking of dietary supplements 114. For instance, in the instant case a provision 128 of a dietary recommendation 126 is limited to six total nutritional products 112. Without forcing the aforementioned substitution, the top six nutritional products 112 would not satisfy the dietary needs of the second user.

Accordingly, a hierarchy of nutritional products 112 having received votes is formed. Based on the assessment responses provided by the second user, the weight of one or more votes cast for various nutritional products is modified. For instance, since the second user indicated in assessment responses that the user does not consume sufficient servings of vegetables each day, votes that are cast for products related to servings of vegetables are increased weighted by a first factor of two due to the dietary patterns of the second user and a second factor of one due to the deficiency. Accordingly, the hierarchy includes the ninth product 112-9 having fifteen votes including one or more votes to address dietary supplement 114 deficiencies of the second user and one or more votes to address health goals of the second user. The one or more votes to address dietary supplement 114 deficiencies includes one vote due to a recommendation that the second user consume less than 2,000 Calories per day, three votes due to a deficiency in vegetable servings (e.g., one vote for the deficiency and two votes for the dietary pattern), three votes due to a deficiency in fruit servings (e.g., one vote for the deficiency and two votes for the dietary pattern), and three votes due to a deficiency in dairy servings (e.g., one vote for the deficiency and two votes for the dietary pattern). The one or more votes to address health goals of the user include three votes due to an energy goal (e.g., one vote for the deficiency and two votes for the medium priority), and two votes due to the sleep goal (e.g., one vote for the deficiency and one votes for the low priority).

The hierarchy of nutritional products 112 further include the second nutritional product 112-2 having eleven total votes including one or more votes to address dietary supplement 114 deficiencies of the second user and one or more votes to address health issues (e.g., the familial history of heart disease) of the second user. The one or more votes to address health issues of the second user includes three votes due to the health issue (e.g., one vote for the deficiency and one vote for the medical practitioner recommendation). The one or more votes to address dietary supplement 114 deficiencies of the user include three votes due a recommended dietary supplement 114 (e.g., one vote for the deficiency and two votes for the dietary pattern). The one or more votes to address health includes include one vote for high cholesterol and one vote for a familial history of high cholesterol, one vote for heart disease and one vote for a familial history heart disease, and one vote for high blood pressure and one vote for a familial high blood pressure.

The hierarchy of nutritional products 112 further include the third nutritional product 112-3 having one or more votes for a dietary supplement 114 deficiency including three votes due to a deficiency in vegetable servings (e.g., one vote for the deficiency and two votes for the dietary pattern), three votes due to a deficiency in fruit servings (e.g., one vote for the deficiency and two votes for the dietary pattern).

The hierarchy of nutritional products 112 further include the fourth nutritional product 112-4 having one or more votes for a dietary supplement 114 deficiency including three votes due to a deficiency in vegetable servings (e.g., one vote for the deficiency and two votes for the dietary pattern), three votes due to a deficiency in fruit servings (e.g., one vote for the deficiency and two votes for the dietary pattern).

The hierarchy of nutritional products 112 further include the fifth nutritional product 112-5 having one or more votes to address health goals of the user include five votes for a stress goal (e.g., one vote for experiencing stress, one for wanting to manage stress, and three votes for stress management being the top priority.)

### EXAMPLE 3: PRESENTING AN ASSESSMENT AND A DIETARY RECOMMENDATION FOR A THIRD USER

Referring briefly to FIGS. 6A through 6H, a third user is provided an assessment survey 162 through a third user device 10-3. The assessment survey 162 includes a plurality of assessment prompts 164. In the present example, the plurality of assessment prompts 164 include a first assessment prompt 164-1 of FIG. 6A, a second assessment prompt 164-2 of FIG. 6B, a third assessment prompt 164-3 of FIG. 6C, and a fourth assessment prompt 164-4, which are presented sequentially to the third user via a display of the third user device 10-3. The third user provides a plurality of assessment responses 132 for some or all of the assessment prompts 164 provided by the assessment survey 162 in order to receive a dietary recommendation 126 (e.g., dietary recommendation 126 of FIGS. 6E through 6H).

The first assessment prompt 164-1 of FIG. 6A is a health goal assessment prompt 164 that elicits a plurality of assessment responses 132 from the third user in order to determine one or more health goals of the third user that the dietary recommendation 164 addresses. Here, the first assessment prompt 164-1 elicits a selection of three assessment responses 132 from eleven assessment responses 132 presented to the third user, which include: a first assessment response 132-1 that corresponds to a first health goal of a weight management health interest; a second assessment response 132-2 that corresponds to a second health goal of a healthy brain health interest; a third assessment response 132-3 that corresponds to a third health goal of a healthy cardiovascular system health interest; a fourth assessment response 132-4 that corresponds to a fourth health goal of a stress management health interest; a fifth assessment response 132-5 that corresponds to a fifth health goal of a healthy immunity system health interest; a sixth assessment response 132-6 that corresponds to a sixth health foal of a healthy digestive system health interest; a seventh assessment response 132-7 that corresponds to a seventh health goal of a healthy joint system health interest; an eight assessment response 132-8 that corresponds to an eight health goal of a healthy sleep health interest; a ninth assessment response 132-9 that corresponds to a ninth health goal of a fitness activity health interest; a tenth assessment response 132-10 that corresponds to a tenth health goal of a health aging health interest; and an eleventh user response 132-11 that corresponds to an eleventh health goal of feeling energic health interest. However, the present disclosure is not so limited. Furthermore, the second assessment prompt 164-2 of FIG. 6B is a lifestyle assessment prompt 164 that elicits an assessment response 132 from the third user in order to determine an aspect of the lifestyle of the third user. Here, the second assessment prompt 164-2 elicits a selection of a respective assessment response 132 to determine a level of physical activity endured by the third user from a plurality of assessment responses 132 presented to the third user, which include a twelfth assessment response 132-12 that corresponds to no physical activity, a thirteenth assessment response 132-13 that corresponds to a moderate level of physical activity, a fourteenth assessment response 132-14 that corresponds to a low level of physical activity, and a eighteenth assessment response 132-15 that corresponds to a high level of physical activity. Here, the third user has selected the fourteenth assessment response 132-14, which is indicated by the check-mark displayed proximate to fourteenth assessment response 132-14, to indicate the low level of physical activity endured by the third user. Additionally, the third assessment prompt 164-3 of FIG. 6C is a lifestyle assessment prompt 164 that elicits a selection of one or more respective assessment responses 132 from a plurality of assessment responses 132 presented to the third users in order to assess a cognitive health of the third user. Here, the plurality of assessment responses 132 presented to the third user via the third assessment prompt 164-3 include a sixteenth assessment response 132-16 that corresponds to an excellent cognitive health assessment, a seventeenth assessment response 132-17 that corresponds to a moderate cognitive health assessment, an eighteenth assessment response 132-18 that corresponds to a good cognitive health assessment, nineteenth assessment response 132-19 that corresponds to a low cognitive health assessment, and a twentieth assessment response 132-20 that corresponds to an adequate cognitive health assessment. In the present example, the third user has selected the third assessment response 132-3 and the fifth assessment response 132-5, indicated by the check-mark displayed proximate to eighteenth assessment response 132-18 and the twentieth assessment response 132-20, respectively, to indicate the good and adequate cognitive health assessments of the third user. Moreover, the fourth assessment prompt 164-4 of FIG. 6D is a physiological assessment prompt 164 that elicits a selection one or more assessment responses 132 associated with whether a health practitioner associated with the third user has indicated a concern associated with the third user for one or more health conditions. Each health condition in the one or more health conditions is associated with a corresponding assessment response 132 from the one or more assessment responses 132 presented to the third user. Here, the one or more assessment responses 132 include a twenty-first assessment response 132-21 that corresponds to an indicated concern for a blood pressure level of the third user, a twenty-second assessment response 132-22 that corresponds to a cholesterol level of the third user, a twenty-third assessment response 132-23 that corresponds to an indicated concern for a weight of the third user, a twenty-fourth assessment response 132-24 that corresponds to an indicated concern for a bone health metric of the third user, a twenty-fifth assessment response 132-25 that corresponds to an indicated concern for a digestion metric of the third user, a twenty-sixth assessment response 132-26 that corresponds to no indicated concern, a twenty-seventh assessment response 132-27 that corresponds to an indicated concern for a blood sugar level of the third user, and a twenty-eighth assessment response 132-28 that corresponds to a joint health of the third user.

Accordingly, these assessment responses 132 from the third user are used with an assessment response 132 to tag 124 lookup structure (e.g., product data store 110, user response data store 130, nutrient data store 150, or a combination thereof) to select a corresponding set of tags 124 for each respective assessment response 132. Collectively, the sets of tags 124 from each respective assessment response 132 form a first plurality of tags 124 associated with the third user identified by the assessment response 132 to tag 124 lookup structure. This first plurality of tags 124 is polled against one or more decision rules 122, which collectively cast votes against a number of nutritional products 112.

The nutritional products 112 are ranked by votes (e.g., ranked using ranker module 140 of FIG. 2B, ranked using ranking engine 320 of FIG. 3, etc.) to determine a hierarchy of nutritional products 112 of the product data store 110. Here, consider the nutritional products 112 that received votes to include: a first nutritional product 112-1 (e.g., first nutritional product 112-1 of FIG. 6F) that addresses one or more dietary deficiencies and a first health problem (e.g., the assessment of adequate cognitive health) of the third user; and a second nutritional product 112-2 (e.g., second nutritional product 112-2 of FIG. 6G) that addresses one or more dietary deficiencies of the second user. Each of the aforementioned respective nutritional products 112 has a corresponding set of tags 124 that collectively form the first plurality of tags 124.

Accordingly, a count of a number of unique tags 124 in the first plurality of tags 124 is determined. In this example, the count of the number of unique tags 124 includes a summation of a number of unique mineral tags 124 and a number of unique vitamin tags 124 (e.g., the count does not include specialty tags 124 and/or functional tags 124).

From this, a hierarchy of nutritional products 112 having received votes is formed. Based on the assessment responses 132 provided by the third user, the weight of one or more votes cast for various nutritional products 112 is modified. For instance, since the third user indicated in assessment responses 132 that the third user does not have an excellent or good cognitive health assessment, votes that are cast for nutritional products 112 related to improving a cognitive health of a user are increased weighted by a first factor. Accordingly, the hierarchy includes the second nutritional product 112-2.

In some embodiments, the hierarchy of nutritional products 112 further include a third nutritional product 112-3 having one or more votes to address health goals of the third user but is not required in a provision of the dietary recommendation 126. In this way, in some embodiments, the dietary recommendation 126 includes a primary dietary recommendation 126-1 (e.g., a dietary recommendation for the third user of the first nutritional product and the second nutritional product 112-2) and one or more auxiliary dietary recommendations 126-2 (e.g., a dietary recommendation for the third user of the first nutritional product 112-1, the second nutritional product 112-2, and the third nutritional product 112-3).

As such, a report (e.g., report 600 of FIGS. 6E through 6H) is provided to the third user and displayed at the third user device 10-3. The report 600 includes the dietary recommendation 126, which includes the aforementioned primary dietary recommendation 126-2 and the auxiliary dietary recommendation 126-2. However, the present disclosure is not so limited. The dietary recommendation 126 includes a recommendation for four different nutritional products 112 (e.g., first nutritional product 112-1 of FIG. 6F, second nutritional product 112-2 of FIG. 6H, . . . , fourth nutritional product 112-4 of FIG. 6E). In some embodiments, the report 600 further includes a dosage of each respective nutritional product 112 in a provision of the dietary recommendation. Here, the dosage is seven total tables consisting of the four different nutritional products 112 of the dietary recommendation. In some embodiments, the report 600 includes a description of one or more assessment responses 132 provided by the third users that form the basis for the dietary recommendation. In some embodiments, the report 600 allows the third user to add or remove a respective nutritional product 112 from the provision of the dietary recommendation 126. In some embodiments, the report 600 includes a detailed description of one or more nutritional products 112 of the dietary recommendation 126, such as a description of one or more tags 124 associated with the one or more nutritional products 112, one or more responses 132 that formed a basis for including the one or more nutritional products 112 (e.g., assessment response 132 of FIG. 6G), one or more dietary supplement 114 associated with the one or more nutritional products 112 (e.g., supplemental facts of FIG. 6H), or a combination thereof. In this way, the report 600 provides detailed information regarding the dietary recommendation 126 provided to a user.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination of FIG. 1, FIG. 2A, or FIG. 2B, and/or described in FIG. 3 through FIG. 5B. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method of providing a dietary recommendation to a subject comprising:
at a computer system comprising at least one processor and a memory storing at least one program for execution by the at least one processor, the at least one program comprising instructions for:
A) obtaining, in electronic form, a plurality of assessment responses for an assessment survey presented to the subject;
B) using each respective assessment response, in all or a subset of the plurality of assessment responses, to select a corresponding set of tags associated with the respective assessment response according to an assessment response to tag lookup data structure, thereby collectively identifying a first plurality of tags;
C) polling the first plurality of tags against a plurality of decision rules, wherein:
each respective decision rule in the plurality of decision rules is independently associated with one or more tags in a second plurality of tags and at least one tag in the second plurality of tags is incorporated into two or more decision rules in the plurality of decision rules,
the second plurality of tags includes all of the tags in the first plurality of tags, and
each time the polling C) determines that the first plurality of tags includes the one or more tags independently associated with a respective decision rule in the plurality of decision rules, the respective decision rule is fired thereby casting a weighted or unweighted vote associated with the respective decision rule against one or more nutritional products in a plurality nutritional products specified by the respective decision rule, the polling C) thereby causing two or more nutritional products in the plurality of nutritional products to have one or more weighted or unweighted votes upon polling all the tags in the first plurality of tags;
D) identifying a subset of the plurality of nutritional products on the basis of the weighted or unweighted votes received by respective nutritional products in the plurality of nutritional products, wherein the subset of the plurality of nutritional products consists of those nutritional products in the plurality of nutritional products that each received a sufficient number of weighted or unweighted votes by the polling C) to satisfy a nutritional product selection criterion;
E) filtering the subset of nutritional products against a plurality of periodic nutritional limits, wherein each respective periodic nutritional limit in the plurality of periodic nutritional limits specifies a corresponding maximum amount of a corresponding dietary supplement that can be consumed in a corresponding period of time given one or more physiological characteristics of the subject specified in the plurality of assessment responses, wherein the filtering independently sums, for each respective dietary supplement associated with each periodic nutritional limit in the plurality of periodic nutritional limits, the corresponding amount of respective dietary supplement in the subset of products and wherein, the filtering E) removes from the subset of nutritional products one or more doses of one or more nutritional products when one or more periodic nutritional limits in the plurality of periodic nutritional limits is determined by the filtering E) to have been exceeded in order to prevent the one or more periodic nutritional limits from being exceeded; and
F) using, after the filtering E), the subset of nutritional products to provide a dietary recommendation to the subject.

2. The method of claim 1, wherein the casted weight either increases a contribution or decreases the contribution of the corresponding one or more nutritional products to satisfy the nutritional product selection criteria.

3. The method of 1, wherein the casted weight against the one or more nutritional products is determined by a first assessment response in the plurality of assessment responses from the subject.

4. The method of claim 1, wherein the identifying D) further comprises:
in accordance with a determination that the subset of the plurality of nutritional products satisfies a threshold periodic nutritional limit, the combination of one or more products comprises a first product.

5. The method of claim 1, wherein the identifying D) further comprises:
in accordance with a determination that the subset of the plurality of nutritional products satisfies a threshold score of unique tags, the subset of the plurality of nutritional products comprises a first product.

6. (canceled)

7. The method of claim 1, wherein the identifying D) further comprises filtering the subset of the plurality of nutritional products based on one or more tags associated with a respective assessment response in the plurality of assessment responses.

8-10. (canceled)

11. The method of claim 1, wherein:
each assessment response in the plurality of assessment responses is obtained from the subject sequentially, thereby forming the plurality of assessment responses, and
after each instance of the obtaining A) a plurality of assessment responses, the method comprises conducting the using B), the polling C), the identifying D), and the filtering E) wherein:
in accordance with a determination that the plurality of assessment responses provided by the subject fail to satisfy the nutritional product selection criterion, repeating the obtaining A); and
in accordance with a determination that the plurality of responses provided by the subject satisfy the threshold criteria, allowing the using F) to provide the dietary recommendation.

12. The method of claim 1, wherein the second plurality of tags further comprise a plurality of vitamin tags, a plurality of mineral tags, a plurality of specialty tags, a plurality of functional tags, or a combination thereof.

13. The method of claim 12, wherein each tag in either of the plurality of vitamin tags or the plurality of mineral tags further comprises a first periodic nutritional limit in the plurality of period nutritional limits that provides (i) a recommended dosage of the corresponding dietary supplement and (ii) a threshold dosage of the corresponding dietary supplement.

14. (canceled)

15. The method of claim 13, wherein the filtering E) further comprises, in accordance with a determination that one or more periodic nutritional limits in the plurality of periodic nutritional limits is exceeded, substituting at least a first product in the subset of the plurality of nutritional products for a second product in the plurality of nutritional products.

16-20. (canceled)

21. The method of claim 1, wherein the assessment survey presented to the subject comprises a plurality of assessment prompts that elicit the one or more physiological characteristics of the subject, the plurality of assessment prompts comprising a plurality of biometric assessment prompts, a plurality of life-stage assessment prompts, a plurality of physiological assessment prompts, a plurality of dietary assessment prompts, a plurality of lifestyle assessment prompts, a plurality of behavioral assessment prompts, a plurality of health goal assessment prompts, or a combination thereof.

22-24. (canceled)

25. The method of claim 21, wherein the plurality of physiological assessment prompts elicits a corresponding plurality of assessment responses comprising:
whether a health practitioner associated with the subject has indicated a concern associated with the subject for one or more health conditions;
a familial health condition history;
whether the health practitioner associated with the subject has indicated a recommendation for one or more dietary supplements; and
whether the subject is currently taking a pharmaceutical composition.

26-30. (canceled)

31. The method of claim 21, wherein the plurality of dietary assessment prompts elicits a corresponding plurality of assessment responses comprising:
whether the subject has one or more dietary restraints;
a number of vegetable servings consumed by the subject;
a number of fruit servings consumed by the subject;
a number of diary servings consumed by the subject; and
a number of omega-3 fatty acid servings consumed by the subject.

32-33. (canceled)

34. The method of claim 21, wherein the plurality of life-style assessment prompts elicits a corresponding plurality of assessment responses comprising:
a level of physical activity endured by the subject;
whether the subject physical exerts themselves at a work;
an energy level of the subject;
a stress level of the subject;
a sleeping habit of the subject;
a cognitive health assessment of the subject;
a sun exposure level of the subject; and
a computer use level of the subject.

35-51. (canceled)

52. The method of claim 1, wherein the filtering E) further comprises determining a target caloric intake of the subject and the nutritional product selection criterion accounts for the target caloric intake of the subject.

53. (canceled)

54. The method of claim 1, wherein the obtaining A) further comprises obtaining a physical location associated with the subject, and wherein the method further comprises:
G) shipping the subset of the plurality of nutritional products of the dietary recommendation to the physical location associated with the subject.

55. (canceled)

56. The method of claim 1, wherein the recommendation includes one or more descriptions, each description providing information related to an opportunity for an improvement in the health of the subject provided by one or more products in the subset of the plurality of nutritional products, or a reason for inclusion of one or more products in the subset of the plurality of nutritional products.

57. The method of claim 1, wherein the recommendation further comprises a dosage regimen for one or more products in the subset of the plurality of nutritional products.

58-60. (canceled)

61. A computer system comprising:
at a computer comprising one or more processors and a memory, the memory comprising non-transitory instructions, which, when executed by the one or more processors, performs a method comprising:
A) obtaining, in electronic form, a plurality of assessment responses for an assessment survey presented to the subject;
B) using each respective assessment response, in all or a subset of the plurality of assessment responses, to select a corresponding set of tags associated with the respective assessment response according to an assessment response to tag lookup data structure, thereby collectively identifying a first plurality of tags;
C) polling the first plurality of tags against a plurality of decision rules, wherein:
each respective decision rule in the plurality of decision rules is independently associated with one or more tags in a second plurality of tags and at least one tag in the second plurality of tags is incorporated into two or more decision rules in the plurality of decision rules,
the second plurality of tags includes all of the tags in the first plurality of tags, and
each time the polling C) determines that the first plurality of tags includes the one or more tags independently associated with a respective decision rule in the plurality of decision rules, the respective decision rule is fired thereby casting a weighted or unweighted vote associated with the respective decision rule against one or more nutritional products in a plurality nutritional products specified by the respective decision rule, the polling C) thereby causing two or more nutritional products in the plurality of nutritional products to have one or more weighted or unweighted votes upon polling all the tags in the first plurality of tags;
D) identifying a subset of the plurality of nutritional products on the basis of the weighted or unweighted votes received by respective nutritional products in the plurality of nutritional products, wherein the subset of the plurality of nutritional products consists of those nutritional products in the plurality of nutritional products that each received a sufficient number of weighted or unweighted votes by the polling C) to satisfy a nutritional product selection criterion;
E) filtering the subset of nutritional products against a plurality of periodic nutritional limits, wherein each respective periodic nutritional limit in the plurality of periodic nutritional limits specifies a corresponding maximum amount of a corresponding dietary supplement that can be consumed in a corresponding period of time given one or more physiological characteristics of the subject specified in the plurality of assessment responses, wherein the filtering independently sums, for each respective dietary supplement associated with each periodic nutritional limit in the plurality of periodic nutritional limits, the corresponding amount of respective dietary supplement in the subset of products and wherein, the filtering E) removes from the subset of nutritional products one or more doses of one or more nutritional products when one or more periodic nutritional limits in the plurality of periodic nutritional limits is determined by the filtering E) to have been exceeded in order to prevent the one or more periodic nutritional limits from being exceeded; and
F) using, after the filtering E), the subset of nutritional products to provide a dietary recommendation to the subject.

62. A non-transitory computer readable storage medium, wherein the non-transitory computer readable storage medium stores instructions, which when executed by a computer system, cause the computer system to perform a method comprising:
A) obtaining, in electronic form, a plurality of assessment responses for an assessment survey presented to the subject;
B) using each respective assessment response, in all or a subset of the plurality of assessment responses, to select a corresponding set of tags associated with the respective assessment response according to an assessment response to tag lookup data structure, thereby collectively identifying a first plurality of tags;
C) polling the first plurality of tags against a plurality of decision rules, wherein:
each respective decision rule in the plurality of decision rules is independently associated with one or more tags in a second plurality of tags and at least one tag in the second plurality of tags is incorporated into two or more decision rules in the plurality of decision rules,
the second plurality of tags includes all of the tags in the first plurality of tags, and
each time the polling C) determines that the first plurality of tags includes the one or more tags independently associated with a respective decision rule in the plurality of decision rules, the respective decision rule is fired thereby casting a weighted or unweighted vote associated with the respective decision rule against one or more nutritional products in a plurality nutritional products specified by the respective decision rule, the polling C) thereby causing two PRELIMINARY AMENDMENT or more nutritional products in the plurality of nutritional products to have one or more weighted or unweighted votes upon polling all the tags in the first plurality of tags;
D) identifying a subset of the plurality of nutritional products on the basis of the weighted or unweighted votes received by respective nutritional products in the plurality of nutritional products, wherein the subset of the plurality of nutritional products consists of those nutritional products in the plurality of nutritional products that each received a sufficient number of weighted or unweighted votes by the polling C) to satisfy a nutritional product selection criterion;
E) filtering the subset of nutritional products against a plurality of periodic nutritional limits, wherein each respective periodic nutritional limit in the plurality of periodic nutritional limits specifies a corresponding maximum amount of a corresponding dietary supplement that can be consumed in a corresponding period of time given one or more physiological characteristics of the subject specified in the plurality of assessment responses, wherein the filtering independently sums, for each respective dietary supplement associated with each periodic nutritional limit in the plurality of periodic nutritional limits, the corresponding amount of respective dietary supplement in the subset of products and wherein, the filtering E) removes from the subset of nutritional products one or more doses of one or more nutritional products when one or more periodic nutritional limits in the plurality of periodic nutritional limits is determined by the filtering E) to have been exceeded in order to prevent the one or more periodic nutritional limits from being exceeded; and
F) using, after the filtering E), the subset of nutritional products to provide a dietary recommendation to the subject.
